(19) United States
(12) Patent Application Publication
(10) Pub. No.: US 2024/0316100 A1
(43) Pub. Date: Sep. 26, 2024

(54) ENGINEERED T CELLS

(71) Applicant: INTELLIA THERAPEUTICS, INC., Cambridge, MA (US)

(72) Inventor: Christopher RUDULIER, Cambridge, MA (US)

(21) Appl. No.: 18/270,261

(22) PCT Filed: Dec. 29, 2021
(86) PCT No.: PCT/US2021/065524

§ 371 Date: Jun. 29, 2023

Related U.S. Application Data
(60) Provisional application No. 63/131,987, filed on Dec. 30, 2020.

(51) Int. Cl. A61K 35/17; A61K 39/00; A61P 37/06; C12N 5/0783; C12N 5/10

(52) U.S. Cl. CPC A61K 35/17; A61K 39/4611; A61K 39/4621; A61K 39/4631; A61K 39/46433; A61K 39/46434; A61K 39/464466; A61P 37/06; C12N 5/0636; C12N 5/10; C12N 2510/00

(57) ABSTRACT

The present disclosure relates to T cells engineered to comprise a modification, e.g., knockdown, of an endogenous nucleic acid sequence encoding an IFNG, a modification, e.g., knockdown, of an endogenous nucleic acid sequence encoding a TNFA, and insertion of sequence(s) encoding a regulatory T cell promoting molecule and compositions and uses thereof.

### SEQUENCE LISTING

The instant application contains a Sequence Listing which has been submitted electronically in ASCII format and is hereby incorporated by reference in its entirety. Said ASCII copy, created on Dec. 28, 2021, is named 12793_0030-00304_SL.txt and is 120,061 bytes in size.

### RELATED APPLICATION

The instant application hereby incorporates by reference U.S. Provisional Application No. 63/131,987, filed on Dec. 30, 2020, the entire contents of which are expressly incorporated herein by reference in their entirety.

### BACKGROUND

Adaptive immunity is a defense mechanism by which the body is able to eliminate foreign pathogens. T cells are immune cells that are capable of mediating this immune response. T cell receptors (TCRs) are protein complexes on the surface of T cells that are capable of recognizing antigens. T cell diversity is derived from rearrangements of TCR alpha and beta loci.

One feature of adaptive immunity is the ability to distinguish “self” from “non-self” antigens. Autoimmune and autoinflammatory disorders are characterized by pathogenic immune responses against “self” antigens. Some rearrangements TCR alpha and beta loci generate self-reactive T cells. Owen et al., Regulatory T Cell Development in the Thymus, J Immunol 203(8) (2019). Many self-reactive T cells are eliminated by clonal deletion in the thymus, but others can escape clonal deletion and elicit deleterious immune responses. Id. Specialized T cells called regulatory T cells (Tregs) are important for “self” tolerance. Id. Tregs are capable of suppressing excessive immune responses, autoimmune responses, and undesired immune responses, for example in graft versus host disease. Id. Dysregulation of Tregs, e.g., if the number of Tregs is insufficient or if Tregs are not functioning properly, may contribute to autoimmune responses. Id.

Current therapies for treating autoimmune disorders aim to suppress the adaptive immune process or the activation of immune cells. While these therapies can suppress deleterious immune responses, e.g., autoimmune responses, they can also suppress beneficial immune responses. Treg therapies have been used to suppress antigen-specific immune responses in different diseases, including graft-versus-host disease (GvHD), in which donor cells mediate an immune attack of host tissues following hematopoietic stem cell transplantation. Pierini et al., T Cells Expressing Chimeric Antigen Receptor Promoter Immune Tolerance, JCI Insight 2(20) (2017). However, there are still “major challenges to the clinical implementation of Treg-based therapies.” Id. Thus, there remains a need for effective T cell therapies, including Treg therapies, for suppressing immune response(s), including inflammation and autoimmunity.

### SUMMARY

The present disclosure provides T cells or a population of T cells engineered to comprise a heterologous nucleic acid encoding a regulatory T cell promoting molecule under control of a promoter sequence; a modification of an endogenous nucleic acid sequence encoding an interferon-gamma (IFNG) wherein the modification knocks down expression of the IFNG; and a modification of an endogenous a nucleic acid sequence encoding a tumor necrosis factor alpha (TNFA) wherein the modification knocks down expression of TNFA, and compositions and uses thereof, e.g., for suppressing immune response(s), including inflammation and autoimmunity. In some embodiments, the regulatory T cell promoting molecule is a selected from interleukin-10 (IL10), cytotoxic T-lymphocyte associated protein 4 (CTLA4), transforming growth factor beta 1 (TGFB1), indoleamine 2,3-dioxygenase 1 (IDO1), ectonucleoside triphosphate diphosphohydrolase 1 (ENTPD1), 5′-nucleotidase ecto (NT5E), interleulin-22 (IL-22), amphiregulin (AREG), interleukin-35 (IL35), GARP, CD274 molecule (CD274), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), and BTB domain and CNC homolog 2 (BACH2).

In some embodiments, the T cells or population of T cells are engineered to comprise a heterologous nucleic acid encoding IL10 under control of a promoter sequence; a modification of an endogenous nucleic acid sequence encoding IFNG wherein the modification knocks down expression of the IFNG; and a modification of an endogenous a nucleic acid sequence encoding TNFA wherein the modification knocks down expression of TNFA.

In some embodiments, the T cells or population of T cells are engineered to comprise a heterologous nucleic acid encoding CTLA4 under control of a promoter sequence; a modification of an endogenous nucleic acid sequence encoding IFNG wherein the modification knocks down expression of the IFNG; and a modification of an endogenous a nucleic acid sequence encoding TNFA wherein the modification knocks down expression of TNFA.

In some embodiments, the T cells or population of T cells are engineered to comprise heterologous nucleic acid sequences encoding IL10 and CTLA4, each under control of a promoter sequence; a modification of an endogenous nucleic acid sequence encoding IFNG wherein the modification knocks down expression of the IFNG; and a modification of an endogenous a nucleic acid sequence encoding TNFA wherein the modification knocks down expression of TNFA.

In some embodiments, the T cells or population of T cells are further engineered to comprise a modification, e.g., knockdown, of an endogenous nucleic acid sequence encoding an interleukin 17A (IL17A), an interleukin-2 (IL2), an interleukin 6 (IL6), a perforin 1 (PRF1), a granzyme A (GZMA), or a granzyme B (GZMB).

In some embodiments, the T cells or population of T cells are further engineered to comprise a modification, e.g., knockdown, of an endogenous nucleic acid sequence encoding an endogenous T cell receptor (TCR).

In some embodiments, the T cells or population of T cells are further engineered to comprise a heterologous coding sequence for a targeting receptor under control of a promoter sequence. In some embodiments, the targeting receptor comprises a chimeric antigen receptor (CAR) or a T cell receptor (TCR). In some embodiments, the targeting receptor is targeted to a ligand selected from mucosal vascular addressin cell adhesion molecule 1 (MADCAM1), tumor necrosis factor alpha (TNFA), CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), desmoglein 3 (DSG3), and MHC class I HLA-A (HLA-A*02).

In some embodiments, at least 30%, 35%, preferably at least 40%, 45%, 50%, 55%, 60%, 65%, 70%, 75%, 80%, 85%, 90%, or 95% of the population of T cells comprises an insertion of the sequence encoding a regulatory T cell promoting molecule, e.g., as assessed by sequencing, e.g., NGS. In some embodiments, at least 50%, 55%, 60%, 65%, preferably at least 70%, 75%, 80%, 85%, 90%, or 95% of the population of T cells comprises a modification, e.g., knockdown, in an IFNG sequence, e.g., as assessed by sequencing, e.g., NGS. In some embodiments, at least 50%, 55%, 60%, 65%, preferably at least 70%, 75%, 80%, 85%, 90%, or 95% of the population of T cells comprises a modification, e.g., knockdown, in an TNFA sequence, e.g., as assessed by sequencing, e.g., NGS. In some embodiments, at least 50%, 55%, 60%, 65%, preferably at least 70%, 75%, 80%, 85%, 90%, or 95% of the population of T cells comprises a modification, e.g., knockdown, in a TCR sequence, e.g., as assessed by sequencing, e.g., NGS. In some embodiments, at least 40%, 45%, 50%, 55%, 60%, 65%, 70%, 75%, 80%, 85%, 90%, or 95% of the population of T cells comprises an insertion of the sequence encoding a targeting receptor, e.g., a CAR, e.g., as assessed by sequencing, e.g., NGS.

The modifications described herein for knocking down expression of a gene may comprise one or more of an insertion, deletion, or substitution. The heterologous sequences described herein may be incorporated into expression construct(s). Multiple heterologous sequences may be incorporated into a single expression construction or into separate expression constructs. The heterologous sequences described herein may be incorporated into episomal expression construct(s). The heterologous sequences described herein may be inserted into the genome, e.g., an untargeted insertion or a targeted insertion. In some embodiments, the targeted insertion is into a site selected from a TCR gene locus, a TNF gene locus, an IFNG gene locus, IL17A gene locus, IL6 gene locus, IL2 gene locus, an adeno-associated virus integration site 1 (AAVS1) locus.

Pharmaceutical compositions and uses of the engineered T cells are also provided herein. In some embodiments, the engineered T cells and pharmaceutical compositions thereof may be administered to a subject in need of immunosuppression. In some embodiments, the engineered T cells and pharmaceutical compositions thereof may be useful in the treatment of an immune disorder or an autoimmune disease, e.g., ulcerative colitis, Crohn's disease, rheumatoid arthritis, psoriasis, multiple sclerosis, systemic lupus erythematosus, type 1 diabetes, and graft versus host disease (GvHD).

In some embodiments, the insertion of sequence(s) or the modification, e.g., knockdown, of sequence(s) described herein may be mediated by guide RNAs in combination with an RNA-guided DNA binding agent, e.g., Cas nuclease. In some embodiments, the insertion of sequence(s) or the knockdown of sequence(s) described herein may be mediated by another suitable gene editing system, e.g., zinc finger nuclease (ZFN) system or transcription activator-like effector nuclease (TALEN) system.

### BRIEF DESCRIPTION OF THE DRAWINGS

FIGS. 1A-1E are histograms of flow cytometry data showing intensity of fluorescence staining of CD3+CD4+ cells, either untransduced or transduced with the insertion of the indicated coding sequences; or in CD3+CD4+CD25+ nTregs. FIG. 1A is a histogram of fluorescence intensity for CTLA expression in transduced T cells or the indicated controls. FIG. 1B is a histogram of fluorescence intensity for IL10 expression in transduced T cells or the indicated controls. FIG. 1C is a histogram of fluorescence intensity for Foxp3 expression in transduced T cells or the indicated controls. FIG. 1D is a histogram of fluorescence intensity for Helios expression in transduced T cells or the indicated controls. FIG. 1E is a histogram of fluorescence intensity for Eos expression in transduced T cells or the indicated controls.

FIGS. 2A and 2B are graphs showing results from a mouse model of GvHD. FIG. 2A is a survival curve showing days of survival following injection with CD3+CD4+ cells transduced with the insertion of the indicated coding sequences or the indicated controls; with CD4+CD25+ Tregs, PBMCs, or not injected with cells (irradiation only). FIG. 2B is a graph showing quantification of human lymphocytes isolated from spleens of mice at the time of sacrifice following injection with transduced CD3+CD4+ cells or controls as indicated.

FIGS. 3A-3E are graphs showing results from the in vitro cytokine profile analysis in stimulated CD3+CD4+ cells transduced with the insertion of the indicated coding sequences or the indicated controls. FIG. 3A shows vitro IL6 production by transduced T cells or the indicated controls upon cell stimulation. FIG. 3B shows in vitro TNF-alpha production by transduced T cells or the indicated controls upon cell stimulation. FIG. 3C shows in vitro IL10 production by transduced T cells or the indicated controls upon cell stimulation. FIG. 3D shows vitro IL13 production by transduced T cells or the indicated controls upon cell stimulation. FIG. 3E shows in vitro IL2 production by transduced T cells or the indicated controls upon cell stimulation. FIG. 3F shows vitro IFN-gamma production by transduced T cells or the indicated controls upon cell stimulation.

FIG. 4 is a graph showing percent suppression of cell proliferation by transduced T cells as measured by CTV dilution in a mixed lymphocyte reaction assay in which CTV labeled T cells and CD-3 depleted PBMC were mixed with the CD3+CD4+ cells transduced with the insertion of the indicated coding sequences or the indicated controls at the indicated ratios.

FIGS. 5A-5E are histograms of flow cytometry data showing intensity of fluorescence staining of CD3+CD4+ cells, either untransduced or transduced with the insertion of the coding sequences of IL10 and CTLA4 alone (no edit) or in combination with a knockout (KO) of one or both of IFNG and TNFA; or in CD3+CD4+CD25+ nTregs. FIG. 5A is a histogram of fluorescence intensity for CTLA4 expression in transduced T cells or the indicated controls. FIG. 5B is a histogram of fluorescence intensity for IL10 expression in transduced T cells or the indicated controls. FIG. 5C is a histogram of fluorescence intensity for FOXP3 expression in transduced T cells or the indicated controls. FIG. 5D is a histogram of fluorescence intensity for Helios expression in transduced T cells or the indicated controls. FIG. 5E is a histogram of fluorescence intensity for Eos expression in transduced T cells or the indicated controls.

FIGS. 6A and 6B are graphs showing results from a mouse model of GvHD. FIG. 6A is a survival curve showing days of survival following injection of mice with PBMC, CD3+CD4+ cells, untransduced or transduced with the insertion of the coding sequences of IL10 and CTLA4 alone (no edit) or in combination with editing (KO) of one or both of IFNG and TNFA; CD3+CD4+CD25+ nTregs; or not injected with cells (irradiated only). FIG. 6B is a graph showing quantification of human lymphocytes isolated from spleens of mice at the time of sacrifice following injection with transduced CD3+CD4+ cells or control cells as indicated.

FIGS. 7A-7F are graphs showing results from the in vitro cytokine profile analysis in stimulated CD3+CD4+ cells either untransduced or transduced with the insertion of coding sequences of IL10 and CTLA4 either alone (no edit) or in combination with editing (KO) of one or both of IFNG or TNFA; or CD4+CD25+ nTregs. FIG. 7A shows vitro IL6 production by transduced T cells or the indicated controls upon cell stimulation. FIG. 7B shows in vitro TNF-alpha production by transduced T cells or the indicated controls upon cell stimulation. FIG. 7C shows in vitro IL10 production by transduced T cells upon cell stimulation. FIG. 7D shows in vitro IL13 production by transduced T cells upon cell stimulation. FIG. 7E shows in vitro IL2 production by transduced T cells upon cell stimulation. FIG. 7F shows in vitro IFN-gamma production by engineered cells upon cell stimulation.

FIG. 8 is a graph showing percent suppression of cell proliferation by engineered T cells as measured by CTV dilution in a mixed lymphocyte reaction assay in which CTV labeled T cells and CD-3 depleted PBMC were mixed with the CD3+CD4+ cells transduced with the insertion of the indicated coding sequences or the indicated controls at the indicated ratios.

FIGS. 9A and 9B are graphs showing results from a mouse model of GvHD. FIG. 9A is a survival curve showing days of survival following injection of mice with PBMC, CD3+CD4+ cells, untransduced or transduced with the insertion of the coding sequences of IL10 and CTLA4, either wild-type (wt) or high affinity (HA), as indicated, in combination with editing (KO) of both IFNG and TNFA; CD3+CD4+CD25+ nTregs; or not injected with cells (vehicle). FIG. 9B is a graph showing quantification of human lymphocytes isolated from spleens of mice at the time of sacrifice following injection with transduced CD3+CD4+ cells or control cells as indicated.

FIGS. 10A and 10B are graphs showing percent suppression of proliferation as measured by CTV dilution in a mixed lymphocyte assay. FIG. 10A show suppression of proliferation, with or without inflammatory preconditioning. FIG. 10B shows suppression of proliferation, with or without inflammatory preconditioning further in the presence of the inflammatory cytokines indicated. The respective p values that are indicated are *p<0.05, **p<0.01, and ***p<0.001.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended embodiments.

The section headings used herein are for organizational purposes only and are not to be construed as limiting the desired subject matter in any way. In the event that any material incorporated by reference contradicts any term defined in this specification or any other express content of this specification, this specification controls.

### I. Definitions

Before describing the present teachings in detail, it is to be understood that the disclosure is not limited to specific compositions or process steps, as such may vary. It should be noted that, as used in this specification and the appended embodiments, the singular form “a”, “an” and “the” include plural references unless the context clearly dictates otherwise. Thus, for example, reference to “a conjugate” includes a plurality of conjugates and reference to “a cell” includes a plurality or population of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or,” unless the context clearly indicates otherwise.

The term “about”, when used before a list or range, modifies each member of the list or each endpoint of the range. The term “about” or “approximately” means an acceptable error for a particular value as determined by one of ordinary skill in the art, which depends in part on how the value is measured or determined. The term “about” is used herein to mean within the typical ranges of tolerances in the art. For example, “about” can be understood as about 2 standard deviations from the mean. In certain embodiments, about means+10%. In certain embodiments, about means+5%.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20 nucleotide nucleic acid molecule” means that 17, 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless stated otherwise, the following terms and phrases as used herein are intended to have the following meanings.

As used herein, “knockdown” refers to a decrease in expression of a particular gene product (e.g., full-length or wild-type mRNA, protein, or both), e.g., in a cell, population of cells, tissue, or organ, by gene editing. In some embodiments, gene editing can be assessed by sequence, e.g., next generation sequencing (NGS). Expression may be decreased by at least 70%, 75%, 80%, 85%, 90%, 95%, or to below the level of detection of the assay as compared to a suitable control, e.g., wherein the gene sequence has not been modified. Knockdown of a protein can be measured by detecting the amount of the protein from a tissue, cell population, or fluid of interest. Methods for measuring knockdown of mRNA are known and include sequencing of mRNA isolated from a tissue or cell population of interest. Flow cytometry analysis is a known method for measuring knockdown of protein expression. For secreted proteins, knockdown may be assessed in a fluid such as tissue culture media or blood, or serum or plasma derived therefrom. In some embodiments, “knockdown” may refer to some loss of expression of a particular gene product, for example a decrease in the amount of full-length, wild-type mRNA transcribed or translated into full-length protein, or a decrease in the amount of protein expressed by a population of cells. It is well understood what changes in an mRNA sequence would result in decreased expression of a wild-type or full-length protein. In some embodiments, “knockdown” may refer to some loss of expression of a particular gene product, for example, an IFNG or TNFA gene product in a body fluid or tissue culture media. A modification of an endogenous nucleic acid sequence, e.g., encoding IFNG or TNFA, may result in a knockdown.

As used herein, “T cell receptor” or “TCR” refers to a receptor in a T cell. In general, a TCR is a heterodimer receptor molecule that contains two TCR polypeptide chains, α and β. α and β chain TCR polypeptides can complex with various CD3 molecules and elicit immune response(s), including inflammation and autoimmunity, after antigen binding. As used herein, a knockdown of TCR refers to a knockdown of any TCR gene in part or in whole, e.g., deletion of part of the TRBC1 gene, alone or in combination with knockdown of other TCR gene(s) in part or in whole.

“TRAC” is used to refer to the T cell receptor α chain. A human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

“TRBC” is used to refer to the T-cell receptor β-chain, e.g., TRBC1 and TRBC2. “TRBC1” and “TRBC2” refer to two homologous genes encoding the T-cell receptor β-chain, which are the gene products of the TRBC1 or TRBC2 genes.

A human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1.

A human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

As used herein, an “immune response” refers to one or more immune system reaction(s), e.g., increased production or activity of immune system cells, such as, but not limited to T cells, B cells, natural killer cells, monocytes, neutrophils, eosinophils, basophils, mast cells, erythrocytes, dendritic cells, antigen presenting cells, macrophages, or phagocytes as compared to an unstimulated control immune system. Exposure of the immune system to an antigen, e.g., a foreign or self-antigen such as but not limited to a pathogen (microorganism, virus, prion, fungus, etc.), an allergen (dust, pollen, dust mite, etc.), a toxin (chemical, drug, etc.), or physiological changes (hypercholesterolemia, obesity, organ transplant, etc.), may cause an immune response. An immune response can also include a response in which donor cells mediate an immune attack of host tissues following hematopoietic stem cell transplantation in GvHD. The immune response may result in inflammation. The immune response may target, attack, remove, or neutralize the antigen, e.g., foreign or self The immune response may or may not be desirable. The immune response may be acute or chronic. The immune response may damage the cell, tissue, or organ against which the immune response is mounted.

As used herein, an “autoimmune response” refers to one or more immune system reaction(s) to a self-antigen, e.g., produced by a subject's own cells, tissues, or organs. The autoimmune response may result in increased production or activity of immune system cells, such as, but not limited to T cells, B cells, natural killer cells, monocytes, neutrophils, eosinophils, basophils, mast cells, erythrocytes, dendritic cells, antigen presenting cells, macrophages, or phagocytes as compared to a suitable control, e.g., a healthy control. The autoimmune response may result in inflammation, e.g., prolonged inflammation, or lead to an autoimmune disease. The autoimmune response may target, attack, remove, or neutralize the self-antigen produced by the subject's own cells, tissues, or organs, which may lead to an autoimmune disease.

As used herein, “suppressing” an immune response(s) refers to decreasing or inhibiting the level of one or more immune system reaction(s), e.g., the production or activity of the immune system cells compared to a suitable control, e.g., not treated with or prior to treatment with the engineered T cell described herein. “Suppressing” an immune response(s) may refer to decreased production or activity of the immune system cells compared to a suitable control, e.g., not treated with or prior to treatment with the engineered T cell described herein. “Suppressing” an immune response may refer to increasing immune tolerance. For example, production or activity of the immune system cells may be measured by cell count, e.g., lymphocyte count or spleen cell count; cell activity, e.g., T cell assay; or gene or protein expression, e.g., biomarker expression; wherein the production or activity is decreased by 50%, 55%, 60%, 65%, 70%, 75%, 80%, 85%, 90%, 95%, or below the level of detection of the assay compared to a suitable control, e.g., not treated with or prior to treatment with the engineered T cell described herein.

As used herein, an “autoimmune disease” or “autoimmune disorder” refers to a condition characterized by pathological immune responses to a subject's own antigens, cells, tissues, or organs. Examples of autoimmune diseases and disorders include, but are not limited to: ulcerative colitis, Crohn's disease, rheumatoid arthritis, psoriasis, multiple sclerosis, systemic lupus erythematosus, and type 1 diabetes. In some embodiments, the engineered T cells have autologous or allogenic use.

As used herein, an “immune disorder” is understood as a disease or condition characterized by a pathological or undesired immune response in a subject. In certain embodiments, an immune disorder is an autoimmune disease. In certain embodiments, an immune disorder is GvHD. In certain embodiments, a subject with an immune disorder is in need of suppression of an immune response. In certain embodiments, a subject with an immune disorder is in need of an increase in immune tolerance.

A “T cell” plays a central role in the immune response following exposure to an antigen. T cells can be naturally occurring or non-natural, e.g., when T cells are formed by engineering, e.g., from a stem cell or by transdifferentiation, e.g., reprogramming a somatic cell. T cells can be distinguished from other lymphocytes by the presence of a T cell receptor on the cell surface. Included in this definition are conventional adaptive T cells, which include helper CD4+ T cells, cytotoxic CD8+ T cells, memory T cells, and regulatory CD4+ T cells, and innate-like T cells including natural killer T cells, mucosal associated invariant T cells, and gamma delta T cells. In some embodiments, T cells are CD4+. In some embodiments, T cells are CD3+/CD4+.

A “regulatory T cell” or “Treg” refers to a specialized T cell that plays a central role in self-tolerance by suppressing excessive immune response(s), including inflammation and autoimmunity. Tregs can be naturally occurring or non-natural, e.g., when Tregs are formed by engineering, e.g., by modifications, e.g., knockdowns, of endogenous nucleic acid sequences encoding IFNG and TNFA and insertion of at least one sequence(s) encoding a regulatory T cell promoting molecule. A naturally occurring Treg or natural Treg or nTreg is a specialized T cell that typically develops in the thymus gland and functions to promote self-tolerance by suppressing excessive immune response(s). In some embodiments, a cell such as a conventional T cell or population of conventional T cells, e.g., a population of T cells not enriched for the presence of nTreg cells, may be engineered by modifying endogenous nucleic sequences encoding TNFA and IFNG, e.g., knocking down nucleic sequences encoding TNFA and IFNG, and insertion of sequence(s) encoding a regulatory T cell promoting molecule into the cell to exhibit the phenotypic characteristics and suppressive functions of a regulatory T cell, and these may be referred to as transduced or “engineered” T cells. In some embodiments, an engineered T cell comprises a modification of an endogenous nucleic acid sequence encoding an IFNG and a modification of an endogenous nucleic acid sequence encoding a TNFA, and insertion of a heterologous regulatory T cell promoting molecule such as IL10 or CTLA4. The modification of an endogenous nucleic acid sequence, e.g., a modification knocks down expression of an endogenous gene, may comprise or consist of one or more indel or substitution mutations in the genomic sequence.

As used herein, “regulatory T cell promoting molecules” refer to molecules that promote the conversion of conventional T cells to regulatory T cells including immunosuppressive molecules and Treg transcription factors. Further, regulatory T cell promoting molecules refer also to molecules that endow conventional T cells with regulatory activity, including Treg-associated immunosuppressive molecules and transcription factors. Examples of immunosuppressive molecules may include, but are not limited to, interleukin-10 (IL10), cytotoxic T-lymphocyte associated protein 4 (CTLA4), transforming growth factor beta 1 (TGFB1), indoleamine 2,3-dioxygenase 1 (IDO1), ectonucleoside triphosphate diphosphohydrolase 1 (ENTPD1), 5′-nucleotidase ecto (NT5E), interleukin-22 (IL22), amphiregulin (AREG), interleukin-35 (IL35), leucine rich repeat containing 32 (GARP), CD274 molecule (CD274), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), and BTB domain and CNC homolog 2 (BACH2). In some embodiments, regulatory T cell promoting molecules may be used in specific combinations, e.g., IL10 and CTLA4, ENTPD1 and NT5E, and IL22 and AREG. In particular, a IL10 and CTLA4 combination is provided herein. In some embodiments, the expression of immunosuppressive molecules may be promoted by the expression of transcription factors such as FoxP3, Helios, Eos, IRF4, Lef1, or BACH2.

In some embodiments, a conventional T cell may be engineered to modify, insert, or delete sequences in the genome, and the “engineered” T cell exhibits one or more phenotypic characteristics and suppressive functions of a natural regulatory T cell. For example, the “engineered” T cell exhibits suppressive activity in a mixed lymphocyte reaction assay as provided in Examples 2 and 3 below, or preferably is capable of inhibiting graft versus host disease in the mouse model presented in Examples 2 and 3 below, preferably in a statistically significant manner (see also, e.g., Parmar et al., Ex vivo fucosylation of third-party human regulatory T cells enhances anti-graft-versus-host disease potency in vivo, Blood 125(9) (2015)). In some embodiments, the “engineered” T cell is a conventional T cell that that has been modified with the insertion of coding sequences for regulatory T cell promoting molecules, and with modification, e.g., knockdown, of expression of pro-inflammatory cytokines, e.g., both IFNG and TNFA. In some embodiments, the starting T cell population for engineering is not enriched for the presence of natural Tregs, e.g., the starting T cell population has less than 20% natural Tregs.

As used herein, a “pro-inflammatory” molecule, e.g., cytokine, increases an immune response as described herein, e.g., reduces the efficacy of a Treg in the mouse model of graft-versus-host disease presented in Examples 2 and 3 in a dose responsive manner. Examples of pro-inflammatory molecules include, but are not limited to, IFNG, TNFA, IL17A, IL6, IL2, perforin 1 (PRF1), granzyme A (GZMA), granzyme B (GZMB).

As used herein, “targeting receptor” refers to a receptor present on the surface of a cell, e.g., a T cell, to permit binding of the cell to a target site, e.g., a specific cell or tissue in an organism. Targeting receptors include, but are not limited to a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion of a protein, e.g., mucosal addressin cell adhesion molecule-1 (MadCAM-1), TNFA, CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), solute carrier family 2 member 2 (SCL2A2), glutamate decarboxylase (GAD2), demoglein 3 (DSG3), and MHC class I HLA-A (HLA-A*02).

As used herein, a “chimeric antigen receptor” refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837, the corresponding portions of the contents of each of which are incorporated herein by reference). A reversed universal CAR that promotes binding of an immune cell to a target cell through an adaptor molecule (see, e.g., WO2019238722, the contents of which are incorporated herein in their entirety) is also contemplated. CARs can be targeted to any antigen to which an antibody can be developed and are typically directed to molecules displayed on the surface of a cell or tissue to be targeted. In some embodiments, the CAR is capable of targeting engineered T cells to the gastrointestinal tract, e.g., the CAR targets MAdCAM-1. In some embodiments, the CAR is capable of targeting engineered T cells to tissues comprising endothelial cells, e.g., the CAR targets VCAM-1, e.g., for suppressing immune responses in disorders such as Crohn's disease and multiple sclerosis. In some embodiments, the CAR is capable of targeting engineered T cells to endothelial cells, e.g., the CAR targets CEACAM6, e.g., for suppressing immune responses in disorders such as Crohn's disease. In some embodiments, the CAR is capable of targeting engineered T cells to pre-B cells, e.g., the CAR targets CD19, e.g., for suppressing immune responses in disorders such as multiple sclerosis and systemic lupus erythematosus. In some embodiments, the CAR is capable of targeting engineered T cells to B lymphocytes, e.g., the CAR targets CD20, e.g., for suppressing immune responses in disorders such as multiple sclerosis and systemic lupus erythematosus. In some embodiments, the CAR is capable of targeting engineered T cells to an inflammatory tissue, e.g., the CAR targets TNFA, e.g., for suppressing immune responses in disorders such as rheumatoid arthritis, inflammatory bowel disease, ulcerative colitis, or Crohn's disease. In some embodiments, the CAR is capable of targeting engineered T cells to an inflammatory tissue, e.g., the CAR targets TGF-b1 e.g., for suppressing immune responses in disorders such as inflammatory bowel disease, ulcerative colitis, or Crohn's disease. In some embodiments, the CAR is capable of targeting engineered T cells to a neurological tissue, e.g., the CAR targets MBP, MOG, or PLP1 e.g., for suppressing immune responses in disorders such as multiple sclerosis. In some embodiments, the CAR is capable of targeting engineered T cells to tissues comprising mature B lymphocytes, e.g., the CAR targets TNFRSF17, e.g., for suppressing immune responses in disorders such as systemic lupus erythematosus. In some embodiments, the CAR is capable of targeting engineered T cells to synovial tissue, e.g., the CAR targets citrullinated vimentin e.g., for suppressing immune responses in disorders such as rheumatoid arthritis. In some embodiments, the CAR targets dipeptidyl peptidase like 6 (DPP6), solute carrier family 2 member 2 (SCL2A2), glutamate decarboxylase (GAD2), demoglein 3 (DSG3), or MHC class I HLA-A (HLA-A*02). Additional CAR targets, e.g., inflammatory antigens, are known in the art. See, e.g., WO2020092057A1, the contents of which are incorporated herein in their entirety.

As used herein, “treatment” refers to any administration or application of a therapeutic for disease or disorder in a subject, and includes inhibiting the disease, arresting its development, relieving one or more symptoms of the disease, curing the disease, preventing one or more symptoms of the disease, or preventing reoccurrence of one or more symptoms of the disease. Treating an autoimmune or inflammatory response or disorder may comprise alleviating the inflammation associated with the specific disorder resulting in the alleviation of disease-specific symptoms. Treatment with the engineered T cells described herein may be used before, after, or in combination with additional therapeutic agents, e.g., anti-inflammatory agents, immunosuppressive agents, or biologics for treatment of autoimmune disorders, e.g., Remicade, Humira.

A “promoter” refers to a regulatory region that controls the expression of a gene to which the regulatory region is linked.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA can have different sugar moieties and can differ by the presence of uracil or analogs thereof in RNA and thymine or analogs thereof in DNA.

“Guide RNA”, “gRNA”, and simply “guide” are used herein interchangeably to refer to either a guide that comprises a guide sequence, e.g., crRNA (also known as CRISPR RNA), or the combination of a crRNA and a trRNA (also known as tracrRNA). The crRNA and trRNA may be associated as a single RNA molecule (single guide RNA, sgRNA) or, for example, in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs, such as sgRNAs or dgRNAs, can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 15, 16, 17, 18, 19, or 20 contiguous nucleotides of a sequence that is complementary to a target. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, or 100%. For example, in some embodiments, the guide sequence comprises a sequence with about 75%, 80%, 85%, 90%, 95%, or 100% identity to at least 15, 16, 17, 18, 19, or 20 contiguous nucleotides of a sequence. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches, or they may be fully complementary, where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse complement), as a nucleic acid substrate for an RNA-guided DNA-binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA-binding agent” means a polypeptide or complex of polypeptides having RNA and DNA binding activity, or a DNA-binding subunit of such a complex, wherein the DNA binding activity is sequence-specific and depends on the sequence of the RNA. The term RNA-guided DNA-binding agent also includes nucleic acids encoding such polypeptides. Exemplary RNA-guided DNA-binding agents include Cas cleavases/nickases. Exemplary RNA-guided DNA-binding agents may include inactivated forms thereof (“dCas DNA-binding agents”), e.g., if those agents are modified to permit DNA cleavage, e.g., via fusion with a FokI cleavase domain. “Cas nuclease”, as used herein, encompasses Cas cleavases and Cas nickases. Cas cleavases and Cas nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated), for example if those agents are modified to permit DNA cleavage, or with a C to T deaminase or A to G deaminase activity. In some embodiments, the RNA-guided DNA-binding agent comprises a deaminase region and an RNA-guided DNA nickase, such as a Cas9 nickase. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), also contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). As used herein, delivery of an RNA-guided DNA-binding agent (e.g., a Cas nuclease, a Cas9 nuclease, or an S. pyogenes Cas9 nuclease) includes delivery of the polypeptide or mRNA.

Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, amino acid sequences, or nucleic acid sequences encoding the amino acid sequences provided herein are also contemplated.

### Exemplary Open Reading Frame for Cas9

(SEQ ID NO: 114)
AUGGACAAGAAGUACUCCAUCGGCCUGGACAUCGGCACCAACUCCGUGG
GCUGGGCCGUGAUCACCGACGAGUACAAGGUGCCCUCCAAGAAGUUCAA
GGUGCUGGGCAACACCGACCGGCACUCCAUCAAGAAGAACCUGAUCGGC
GCCCUGCUGUUCGACUCCGGCGAGACCGCCGAGGCCACCCGGCUGAAGC
GGACCGCCCGGCGGCGGUACACCCGGCGGAAGAACCGGAUCUGCUACCU
GCAGGAGAUCUUCUCCAACGAGAUGGCCAAGGUGGACGACUCCUUCUUC
CACCGGCUGGAGGAGUCCUUCCUGGUGGAGGAGGACAAGAAGCACGAGC
GGCACCCCAUCUUCGGCAACAUCGUGGACGAGGUGGCCUACCACGAGAA
GUACCCCACCAUCUACCACCUGCGGAAGAAGCUGGUGGACUCCACCGAC
AAGGCCGACCUGCGGCUGAUCUACCUGGCCCUGGCCCACAUGAUCAAGU
UCCGGGGCCACUUCCUGAUCGAGGGCGACCUGAACCCCGACAACUCCGA
CGUGGACAAGCUGUUCAUCCAGCUGGUGCAGACCUACAACCAGCUGUUC
GAGGAGAACCCCAUCAACGCCUCCGGCGUGGACGCCAAGGCCAUCCUGU
CCGCCCGGCUGUCCAAGUCCCGGCGGCUGGAGAACCUGAUCGCCCAGCU
GCCCGGCGAGAAGAAGAACGGCCUGUUCGGCAACCUGAUCGCCCUGUCC
CUGGGCCUGACCCCCAACUUCAAGUCCAACUUCGACCUGGCCGAGGACG
CCAAGCUGCAGCUGUCCAAGGACACCUACGACGACGACCUGGACAACCU
GCUGGCCCAGAUCGGCGACCAGUACGCCGACCUGUUCCUGGCCGCCAAG
AACCUGUCCGACGCCAUCCUGCUGUCCGACAUCCUGCGGGUGAACACCG
AGAUCACCAAGGCCCCCCUGUCCGCCUCCAUGAUCAAGCGGUACGACGA
GCACCACCAGGACCUGACCCUGCUGAAGGCCCUGGUGCGGCAGCAGCUG
CCCGAGAAGUACAAGGAGAUCUUCUUCGACCAGUCCAAGAACGGCUACG
CCGGCUACAUCGACGGCGGCGCCUCCCAGGAGGAGUUCUACAAGUUCAU
CAAGCCCAUCCUGGAGAAGAUGGACGGCACCGAGGAGCUGCUGGUGAAG
CUGAACCGGGAGGACCUGCUGCGGAAGCAGCGGACCUUCGACAACGGCU
CCAUCCCCCACCAGAUCCACCUGGGCGAGCUGCACGCCAUCCUGCGGCG
GCAGGAGGACUUCUACCCCUUCCUGAAGGACAACCGGGAGAAGAUCGAG
AAGAUCCUGACCUUCCGGAUCCCCUACUACGUGGGCCCCCUGGCCCGGG
GCAACUCCCGGUUCGCCUGGAUGACCCGGAAGUCCGAGGAGACCAUCAC
CCCCUGGAACUUCGAGGAGGUGGUGGACAAGGGCGCCUCCGCCCAGUCC
UUCAUCGAGCGGAUGACCAACUUCGACAAGAACCUGCCCAACGAGAAGG
UGCUGCCCAAGCACUCCCUGCUGUACGAGUACUUCACCGUGUACAACGA
GCUGACCAAGGUGAAGUACGUGACCGAGGGCAUGCGGAAGCCCGCCUUC
CUGUCCGGCGAGCAGAAGAAGGCCAUCGUGGACCUGCUGUUCAAGACCA
ACCGGAAGGUGACCGUGAAGCAGCUGAAGGAGGACUACUUCAAGAAGAU
CGAGUGCUUCGACUCCGUGGAGAUCUCCGGCGUGGAGGACCGGUUCAAC
GCCUCCCUGGGCACCUACCACGACCUGCUGAAGAUCAUCAAGGACAAGG
ACUUCCUGGACAACGAGGAGAACGAGGACAUCCUGGAGGACAUCGUGCU
GACCCUGACCCUGUUCGAGGACCGGGAGAUGAUCGAGGAGCGGCUGAAG
ACCUACGCCCACCUGUUCGACGACAAGGUGAUGAAGCAGCUGAAGCGGC
GGCGGUACACCGGCUGGGGCCGGCUGUCCCGGAAGCUGAUCAACGGCAU
CCGGGACAAGCAGUCCGGCAAGACCAUCCUGGACUUCCUGAAGUCCGAC
GGCUUCGCCAACCGGAACUUCAUGCAGCUGAUCCACGACGACUCCCUGA
CCUUCAAGGAGGACAUCCAGAAGGCCCAGGUGUCCGGCCAGGGCGACUC
CCUGCACGAGCACAUCGCCAACCUGGCCGGCUCCCCCGCCAUCAAGAAG
GGCAUCCUGCAGACCGUGAAGGUGGUGGACGAGCUGGUGAAGGUGAUGG
GCCGGCACAAGCCCGAGAACAUCGUGAUCGAGAUGGCCCGGGAGAACCA
GACCACCCAGAAGGGCCAGAAGAACUCCCGGGAGCGGAUGAAGCGGAUC
GAGGAGGGCAUCAAGGAGCUGGGCUCCCAGAUCCUGAAGGAGCACCCCG
UGGAGAACACCCAGCUGCAGAACGAGAAGCUGUACCUGUACUACCUGCA
GAACGGCCGGGACAUGUACGUGGACCAGGAGCUGGACAUCAACCGGCUG
UCCGACUACGACGUGGACCACAUCGUGCCCCAGUCCUUCCUGAAGGACG
ACUCCAUCGACAACAAGGUGCUGACCCGGUCCGACAAGAACCGGGGCAA
GUCCGACAACGUGCCCUCCGAGGAGGUGGUGAAGAAGAUGAAGAACUAC
UGGCGGCAGCUGCUGAACGCCAAGCUGAUCACCCAGCGGAAGUUCGACA
ACCUGACCAAGGCCGAGCGGGGCGGCCUGUCCGAGCUGGACAAGGCCGG
CUUCAUCAAGCGGCAGCUGGUGGAGACCCGGCAGAUCACCAAGCACGUG
GCCCAGAUCCUGGACUCCCGGAUGAACACCAAGUACGACGAGAACGACA
AGCUGAUCCGGGAGGUGAAGGUGAUCACCCUGAAGUCCAAGCUGGUGUC
CGACUUCCGGAAGGACUUCCAGUUCUACAAGGUGCGGGAGAUCAACAAC
UACCACCACGCCCACGACGCCUACCUGAACGCCGUGGUGGGCACCGCCC
UGAUCAAGAAGUACCCCAAGCUGGAGUCCGAGUUCGUGUACGGCGACUA
CAAGGUGUACGACGUGCGGAAGAUGAUCGCCAAGUCCGAGCAGGAGAUC
GGCAAGGCCACCGCCAAGUACUUCUUCUACUCCAACAUCAUGAACUUCU
UCAAGACCGAGAUCACCCUGGCCAACGGCGAGAUCCGGAAGCGGCCCCU
GAUCGAGACCAACGGCGAGACCGGCGAGAUCGUGUGGGACAAGGGCCGG
GACUUCGCCACCGUGCGGAAGGUGCUGUCCAUGCCCCAGGUGAACAUCG
UGAAGAAGACCGAGGUGCAGACCGGCGGCUUCUCCAAGGAGUCCAUCCU
GCCCAAGCGGAACUCCGACAAGCUGAUCGCCCGGAAGAAGGACUGGGAC
CCCAAGAAGUACGGCGGCUUCGACUCCCCCACCGUGGCCUACUCCGUGC
UGGUGGUGGCCAAGGUGGAGAAGGGCAAGUCCAAGAAGCUGAAGUCCGU
GAAGGAGCUGCUGGGCAUCACCAUCAUGGAGCGGUCCUCCUUCGAGAAG
AACCCCAUCGACUUCCUGGAGGCCAAGGGCUACAAGGAGGUGAAGAAGG
ACCUGAUCAUCAAGCUGCCCAAGUACUCCCUGUUCGAGCUGGAGAACGG
CCGGAAGCGGAUGCUGGCCUCCGCCGGCGAGCUGCAGAAGGGCAACGAG
CUGGCCCUGCCCUCCAAGUACGUGAACUUCCUGUACCUGGCCUCCCACU
ACGAGAAGCUGAAGGGCUCCCCCGAGGACAACGAGCAGAAGCAGCUGUU
CGUGGAGCAGCACAAGCACUACCUGGACGAGAUCAUCGAGCAGAUCUCC
GAGUUCUCCAAGCGGGUGAUCCUGGCCGACGCCAACCUGGACAAGGUGC
UGUCCGCCUACAACAAGCACCGGGACAAGCCCAUCCGGGAGCAGGCCGA
GAACAUCAUCCACCUGUUCACCCUGACCAACCUGGGCGCCCCCGCCGCC
UUCAAGUACUUCGACACCACCAUCGACCGGAAGCGGUACACCUCCACCA
AGGAGGUGCUGGACGCCACCCUGAUCCACCAGUCCAUCACCGGCCUGUA
CGAGACCCGGAUCGACCUGUCCCAGCUGGGCGGCGACGGCGGCGGCUCC
CCCAAGAAGAAGCGGAAGGUGUGA

### Exemplary Amino Acid Sequence for Cas9

(SEQ ID NO: 115)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGDGGGS
PKKKRKV

### Exemplary Open Reading Frame for Cas9

(SEQ ID NO: 116)
AUGGACAAGAAGUACAGCAUCGGACUGGACAUCGGAACAAACAGCGUCG
GAUGGGCAGUCAUCACAGACGAAUACAAGGUCCCGAGCAAGAAGUUCAA
GGUCCUGGGAAACACAGACAGACACAGCAUCAAGAAGAACCUGAUCGGA
GCACUGCUGUUCGACAGCGGAGAAACAGCAGAAGCAACAAGACUGAAGA
GAACAGCAAGAAGAAGAUACACAAGAAGAAAGAACAGAAUCUGCUACCU
GCAGGAAAUCUUCAGCAACGAAAUGGCAAAGGUCGACGACAGCUUCUUC
CACAGACUGGAAGAAAGCUUCCUGGUCGAAGAAGACAAGAAGCACGAAA
GACACCCGAUCUUCGGAAACAUCGUCGACGAAGUCGCAUACCACGAAAA
GUACCCGACAAUCUACCACCUGAGAAAGAAGCUGGUCGACAGCACAGAC
AAGGCAGACCUGAGACUGAUCUACCUGGCACUGGCACACAUGAUCAAGU
UCAGAGGACACUUCCUGAUCGAAGGAGACCUGAACCCGGACAACAGCGA
CGUCGACAAGCUGUUCAUCCAGCUGGUCCAGACAUACAACCAGCUGUUC
GAAGAAAACCCGAUCAACGCAAGCGGAGUCGACGCAAAGGCAAUCCUGA
GCGCAAGACUGAGCAAGAGCAGAAGACUGGAAAACCUGAUCGCACAGCU
GCCGGGAGAAAAGAAGAACGGACUGUUCGGAAACCUGAUCGCACUGAGC
CUGGGACUGACACCGAACUUCAAGAGCAACUUCGACCUGGCAGAAGACG
CAAAGCUGCAGCUGAGCAAGGACACAUACGACGACGACCUGGACAACCU
GCUGGCACAGAUCGGAGACCAGUACGCAGACCUGUUCCUGGCAGCAAAG
AACCUGAGCGACGCAAUCCUGCUGAGCGACAUCCUGAGAGUCAACACAG
AAAUCACAAAGGCACCGCUGAGCGCAAGCAUGAUCAAGAGAUACGACGA
ACACCACCAGGACCUGACACUGCUGAAGGCACUGGUCAGACAGCAGCUG
CCGGAAAAGUACAAGGAAAUCUUCUUCGACCAGAGCAAGAACGGAUACG
CAGGAUACAUCGACGGAGGAGCAAGCCAGGAAGAAUUCUACAAGUUCAU
CAAGCCGAUCCUGGAAAAGAUGGACGGAACAGAAGAACUGCUGGUCAAG
CUGAACAGAGAAGACCUGCUGAGAAAGCAGAGAACAUUCGACAACGGAA
GCAUCCCGCACCAGAUCCACCUGGGAGAACUGCACGCAAUCCUGAGAAG
ACAGGAAGACUUCUACCCGUUCCUGAAGGACAACAGAGAAAAGAUCGAA
AAGAUCCUGACAUUCAGAAUCCCGUACUACGUCGGACCGCUGGCAAGAG
GAAACAGCAGAUUCGCAUGGAUGACAAGAAAGAGCGAAGAAACAAUCAC
ACCGUGGAACUUCGAAGAAGUCGUCGACAAGGGAGCAAGCGCACAGAGC
UUCAUCGAAAGAAUGACAAACUUCGACAAGAACCUGCCGAACGAAAAGG
UCCUGCCGAAGCACAGCCUGCUGUACGAAUACUUCACAGUCUACAACGA
ACUGACAAAGGUCAAGUACGUCACAGAAGGAAUGAGAAAGCCGGCAUUC
CUGAGCGGAGAACAGAAGAAGGCAAUCGUCGACCUGCUGUUCAAGACAA
ACAGAAAGGUCACAGUCAAGCAGCUGAAGGAAGACUACUUCAAGAAGAU
CGAAUGCUUCGACAGCGUCGAAAUCAGCGGAGUCGAAGACAGAUUCAAC
GCAAGCCUGGGAACAUACCACGACCUGCUGAAGAUCAUCAAGGACAAGG
ACUUCCUGGACAACGAAGAAAACGAAGACAUCCUGGAAGACAUCGUCCU
GACACUGACACUGUUCGAAGACAGAGAAAUGAUCGAAGAAAGACUGAAG
ACAUACGCACACCUGUUCGACGACAAGGUCAUGAAGCAGCUGAAGAGAA
GAAGAUACACAGGAUGGGGAAGACUGAGCAGAAAGCUGAUCAACGGAAU
CAGAGACAAGCAGAGCGGAAAGACAAUCCUGGACUUCCUGAAGAGCGAC
GGAUUCGCAAACAGAAACUUCAUGCAGCUGAUCCACGACGACAGCCUGA
CAUUCAAGGAAGACAUCCAGAAGGCACAGGUCAGCGGACAGGGAGACAG
CCUGCACGAACACAUCGCAAACCUGGCAGGAAGCCCGGCAAUCAAGAAG
GGAAUCCUGCAGACAGUCAAGGUCGUCGACGAACUGGUCAAGGUCAUGG
GAAGACACAAGCCGGAAAACAUCGUCAUCGAAAUGGCAAGAGAAAACCA
GACAACACAGAAGGGACAGAAGAACAGCAGAGAAAGAAUGAAGAGAAUC
GAAGAAGGAAUCAAGGAACUGGGAAGCCAGAUCCUGAAGGAACACCCGG
UCGAAAACACACAGCUGCAGAACGAAAAGCUGUACCUGUACUACCUGCA
GAACGGAAGAGACAUGUACGUCGACCAGGAACUGGACAUCAACAGACUG
AGCGACUACGACGUCGACCACAUCGUCCCGCAGAGCUUCCUGAAGGACG
ACAGCAUCGACAACAAGGUCCUGACAAGAAGCGACAAGAACAGAGGAAA
GAGCGACAACGUCCCGAGCGAAGAAGUCGUCAAGAAGAUGAAGAACUAC
UGGAGACAGCUGCUGAACGCAAAGCUGAUCACACAGAGAAAGUUCGACA
ACCUGACAAAGGCAGAGAGAGGAGGACUGAGCGAACUGGACAAGGCAGG
AUUCAUCAAGAGACAGCUGGUCGAAACAAGACAGAUCACAAAGCACGUC
GCACAGAUCCUGGACAGCAGAAUGAACACAAAGUACGACGAAAACGACA
AGCUGAUCAGAGAAGUCAAGGUCAUCACACUGAAGAGCAAGCUGGUCAG
CGACUUCAGAAAGGACUUCCAGUUCUACAAGGUCAGAGAAAUCAACAAC
UACCACCACGCACACGACGCAUACCUGAACGCAGUCGUCGGAACAGCAC
UGAUCAAGAAGUACCCGAAGCUGGAAAGCGAAUUCGUCUACGGAGACUA
CAAGGUCUACGACGUCAGAAAGAUGAUCGCAAAGAGCGAACAGGAAAUC
GGAAAGGCAACAGCAAAGUACUUCUUCUACAGCAACAUCAUGAACUUCU
UCAAGACAGAAAUCACACUGGCAAACGGAGAAAUCAGAAAGAGACCGCU
GAUCGAAACAAACGGAGAAACAGGAGAAAUCGUCUGGGACAAGGGAAGA
GACUUCGCAACAGUCAGAAAGGUCCUGAGCAUGCCGCAGGUCAACAUCG
UCAAGAAGACAGAAGUCCAGACAGGAGGAUUCAGCAAGGAAAGCAUCCU
GCCGAAGAGAAACAGCGACAAGCUGAUCGCAAGAAAGAAGGACUGGGAC
CCGAAGAAGUACGGAGGAUUCGACAGCCCGACAGUCGCAUACAGCGUCC
UGGUCGUCGCAAAGGUCGAAAAGGGAAAGAGCAAGAAGCUGAAGAGCGU
CAAGGAACUGCUGGGAAUCACAAUCAUGGAAAGAAGCAGCUUCGAAAAG
AACCCGAUCGACUUCCUGGAAGCAAAGGGAUACAAGGAAGUCAAGAAGG
ACCUGAUCAUCAAGCUGCCGAAGUACAGCCUGUUCGAACUGGAAAACGG
AAGAAAGAGAAUGCUGGCAAGCGCAGGAGAACUGCAGAAGGGAAACGAA
CUGGCACUGCCGAGCAAGUACGUCAACUUCCUGUACCUGGCAAGCCACU
ACGAAAAGCUGAAGGGAAGCCCGGAAGACAACGAACAGAAGCAGCUGUU
CGUCGAACAGCACAAGCACUACCUGGACGAAAUCAUCGAACAGAUCAGC
GAAUUCAGCAAGAGAGUCAUCCUGGCAGACGCAAACCUGGACAAGGUCC
UGAGCGCAUACAACAAGCACAGAGACAAGCCGAUCAGAGAACAGGCAGA
AAACAUCAUCCACCUGUUCACACUGACAAACCUGGGAGCACCGGCAGCA
UUCAAGUACUUCGACACAACAAUCGACAGAAAGAGAUACACAAGCACAA
AGGAAGUCCUGGACGCAACACUGAUCCACCAGAGCAUCACAGGACUGUA
CGAAACAAGAAUCGACCUGAGCCAGCUGGGAGGAGACGGAGGAGGAAGC
CCGAAGAAGAAGAGAAAGGUCUAG

Exemplary Open Reading Frame for Cas9 with Hibit Tag

(SEQ ID NO: 117)
AUGGACAAGAAGUACUCCAUCGGCCUGGACAUCGGCACCAACUCCGUGG
GCUGGGCCGUGAUCACCGACGAGUACAAGGUGCCCUCCAAGAAGUUCAA
GGUGCUGGGCAACACCGACCGGCACUCCAUCAAGAAGAACCUGAUCGGC
GCCCUGCUGUUCGACUCCGGCGAGACCGCCGAGGCCACCCGGCUGAAGC
GGACCGCCCGGCGGCGGUACACCCGGCGGAAGAACCGGAUCUGCUACCU
GCAGGAGAUCUUCUCCAACGAGAUGGCCAAGGUGGACGACUCCUUCUUC
CACCGGCUGGAGGAGUCCUUCCUGGUGGAGGAGGACAAGAAGCACGAGC
GGCACCCCAUCUUCGGCAACAUCGUGGACGAGGUGGCCUACCACGAGAA
GUACCCCACCAUCUACCACCUGCGGAAGAAGCUGGUGGACUCCACCGAC
AAGGCCGACCUGCGGCUGAUCUACCUGGCCCUGGCCCACAUGAUCAAGU
UCCGGGGCCACUUCCUGAUCGAGGGCGACCUGAACCCCGACAACUCCGA
CGUGGACAAGCUGUUCAUCCAGCUGGUGCAGACCUACAACCAGCUGUUC
GAGGAGAACCCCAUCAACGCCUCCGGCGUGGACGCCAAGGCCAUCCUGU
CCGCCCGGCUGUCCAAGUCCCGGCGGCUGGAGAACCUGAUCGCCCAGCU
GCCCGGCGAGAAGAAGAACGGCCUGUUCGGCAACCUGAUCGCCCUGUCC
CUGGGCCUGACCCCCAACUUCAAGUCCAACUUCGACCUGGCCGAGGACG
CCAAGCUGCAGCUGUCCAAGGACACCUACGACGACGACCUGGACAACCU
GCUGGCCCAGAUCGGCGACCAGUACGCCGACCUGUUCCUGGCCGCCAAG
AACCUGUCCGACGCCAUCCUGCUGUCCGACAUCCUGCGGGUGAACACCG
AGAUCACCAAGGCCCCCCUGUCCGCCUCCAUGAUCAAGCGGUACGACGA
GCACCACCAGGACCUGACCCUGCUGAAGGCCCUGGUGCGGCAGCAGCUG
CCCGAGAAGUACAAGGAGAUCUUCUUCGACCAGUCCAAGAACGGCUACG
CCGGCUACAUCGACGGCGGCGCCUCCCAGGAGGAGUUCUACAAGUUCAU
CAAGCCCAUCCUGGAGAAGAUGGACGGCACCGAGGAGCUGCUGGUGAAG
CUGAACCGGGAGGACCUGCUGCGGAAGCAGCGGACCUUCGACAACGGCU
CCAUCCCCCACCAGAUCCACCUGGGCGAGCUGCACGCCAUCCUGCGGCG
GCAGGAGGACUUCUACCCCUUCCUGAAGGACAACCGGGAGAAGAUCGAG
AAGAUCCUGACCUUCCGGAUCCCCUACUACGUGGGCCCCCUGGCCCGGG
GCAACUCCCGGUUCGCCUGGAUGACCCGGAAGUCCGAGGAGACCAUCAC
CCCCUGGAACUUCGAGGAGGUGGUGGACAAGGGCGCCUCCGCCCAGUCC
UUCAUCGAGCGGAUGACCAACUUCGACAAGAACCUGCCCAACGAGAAGG
UGCUGCCCAAGCACUCCCUGCUGUACGAGUACUUCACCGUGUACAACGA
GCUGACCAAGGUGAAGUACGUGACCGAGGGCAUGCGGAAGCCCGCCUUC
CUGUCCGGCGAGCAGAAGAAGGCCAUCGUGGACCUGCUGUUCAAGACCA
ACCGGAAGGUGACCGUGAAGCAGCUGAAGGAGGACUACUUCAAGAAGAU
CGAGUGCUUCGACUCCGUGGAGAUCUCCGGCGUGGAGGACCGGUUCAAC
GCCUCCCUGGGCACCUACCACGACCUGCUGAAGAUCAUCAAGGACAAGG
ACUUCCUGGACAACGAGGAGAACGAGGACAUCCUGGAGGACAUCGUGCU
GACCCUGACCCUGUUCGAGGACCGGGAGAUGAUCGAGGAGCGGCUGAAG
ACCUACGCCCACCUGUUCGACGACAAGGUGAUGAAGCAGCUGAAGCGGC
GGCGGUACACCGGCUGGGGCCGGCUGUCCCGGAAGCUGAUCAACGGCAU
CCGGGACAAGCAGUCCGGCAAGACCAUCCUGGACUUCCUGAAGUCCGAC
GGCUUCGCCAACCGGAACUUCAUGCAGCUGAUCCACGACGACUCCCUGA
CCUUCAAGGAGGACAUCCAGAAGGCCCAGGUGUCCGGCCAGGGCGACUC
CCUGCACGAGCACAUCGCCAACCUGGCCGGCUCCCCCGCCAUCAAGAAG
GGCAUCCUGCAGACCGUGAAGGUGGUGGACGAGCUGGUGAAGGUGAUGG
GCCGGCACAAGCCCGAGAACAUCGUGAUCGAGAUGGCCCGGGAGAACCA
GACCACCCAGAAGGGCCAGAAGAACUCCCGGGAGCGGAUGAAGCGGAUC
GAGGAGGGCAUCAAGGAGCUGGGCUCCCAGAUCCUGAAGGAGCACCCCG
UGGAGAACACCCAGCUGCAGAACGAGAAGCUGUACCUGUACUACCUGCA
GAACGGCCGGGACAUGUACGUGGACCAGGAGCUGGACAUCAACCGGCUG
UCCGACUACGACGUGGACCACAUCGUGCCCCAGUCCUUCCUGAAGGACG
ACUCCAUCGACAACAAGGUGCUGACCCGGUCCGACAAGAACCGGGGCAA
GUCCGACAACGUGCCCUCCGAGGAGGUGGUGAAGAAGAUGAAGAACUAC
UGGCGGCAGCUGCUGAACGCCAAGCUGAUCACCCAGCGGAAGUUCGACA
ACCUGACCAAGGCCGAGCGGGGCGGCCUGUCCGAGCUGGACAAGGCCGG
CUUCAUCAAGCGGCAGCUGGUGGAGACCCGGCAGAUCACCAAGCACGUG
GCCCAGAUCCUGGACUCCCGGAUGAACACCAAGUACGACGAGAACGACA
AGCUGAUCCGGGAGGUGAAGGUGAUCACCCUGAAGUCCAAGCUGGUGUC
CGACUUCCGGAAGGACUUCCAGUUCUACAAGGUGCGGGAGAUCAACAAC
UACCACCACGCCCACGACGCCUACCUGAACGCCGUGGUGGGCACCGCCC
UGAUCAAGAAGUACCCCAAGCUGGAGUCCGAGUUCGUGUACGGCGACUA
CAAGGUGUACGACGUGCGGAAGAUGAUCGCCAAGUCCGAGCAGGAGAUC
GGCAAGGCCACCGCCAAGUACUUCUUCUACUCCAACAUCAUGAACUUCU
UCAAGACCGAGAUCACCCUGGCCAACGGCGAGAUCCGGAAGCGGCCCCU
GAUCGAGACCAACGGCGAGACCGGCGAGAUCGUGUGGGACAAGGGCCGG
GACUUCGCCACCGUGCGGAAGGUGCUGUCCAUGCCCCAGGUGAACAUCG
UGAAGAAGACCGAGGUGCAGACCGGCGGCUUCUCCAAGGAGUCCAUCCU
GCCCAAGCGGAACUCCGACAAGCUGAUCGCCCGGAAGAAGGACUGGGAC
CCCAAGAAGUACGGCGGCUUCGACUCCCCCACCGUGGCCUACUCCGUGC
UGGUGGUGGCCAAGGUGGAGAAGGGCAAGUCCAAGAAGCUGAAGUCCGU
GAAGGAGCUGCUGGGCAUCACCAUCAUGGAGCGGUCCUCCUUCGAGAAG
AACCCCAUCGACUUCCUGGAGGCCAAGGGCUACAAGGAGGUGAAGAAGG
ACCUGAUCAUCAAGCUGCCCAAGUACUCCCUGUUCGAGCUGGAGAACGG
CCGGAAGCGGAUGCUGGCCUCCGCCGGCGAGCUGCAGAAGGGCAACGAG
CUGGCCCUGCCCUCCAAGUACGUGAACUUCCUGUACCUGGCCUCCCACU
ACGAGAAGCUGAAGGGCUCCCCCGAGGACAACGAGCAGAAGCAGCUGUU
CGUGGAGCAGCACAAGCACUACCUGGACGAGAUCAUCGAGCAGAUCUCC
GAGUUCUCCAAGCGGGUGAUCCUGGCCGACGCCAACCUGGACAAGGUGC
UGUCCGCCUACAACAAGCACCGGGACAAGCCCAUCCGGGAGCAGGCCGA
GAACAUCAUCCACCUGUUCACCCUGACCAACCUGGGCGCCCCCGCCGCC
UUCAAGUACUUCGACACCACCAUCGACCGGAAGCGGUACACCUCCACCA
AGGAGGUGCUGGACGCCACCCUGAUCCACCAGUCCAUCACCGGCCUGUA
CGAGACCCGGAUCGACCUGUCCCAGCUGGGCGGCGACGGCGGCGGCUCC
CCCAAGAAGAAGCGGAAGGUGUCCGAGUCCGCCACCCCCGAGUCCGUGU
CCGGCUGGCGGCUGUUCAAGAAGAUCUCCUGA

Exemplary Amino Acid Sequence for Cas9 with Hibit Tag

(SEQ ID NO: 118)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGDGGGS
PKKKRKVSESATPESVSGWRLFKKIS*

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA-binding agent such as Cas9 to a target sequence, and the guide RNA hybridizes with and the agent binds to the target sequence; in cases where the agent is a cleavase or nickase, binding can be followed by double-stranded DNA cleavage or single-stranded DNA cleavage.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, a first sequence is considered to be “X % complementary to” a second sequence if X % of the bases of the first sequence base pairs with the second sequence. For example, a first sequence 5′AAGA3′ is 100% complementary to a second sequence 3′TTCT5′, and the second sequence is 100% complementary to the first sequence. In some embodiments, a first sequence 5′AAGA3′ is 100% complementary to a second sequence 3′TTCTGTGA5′, whereas the second sequence is 50% complementary to the first sequence.

As used herein, “mRNA” is used herein to refer to a polynucleotide that is entirely or predominantly RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indel” refers to insertion/deletion mutations consisting of a number of nucleotides that are either inserted or deleted at the site of a double-stranded break (DSB) in a target nucleic acid.

As used herein, a “target sequence” refers to a sequence of nucleic acid in a target gene that has complementarity to the guide sequence of the gRNA. The interaction of the target sequence and the guide sequence directs an RNA-guided DNA-binding agent to bind, and potentially nick or cleave (depending on the activity of the agent), within the target sequence.

As used herein, “polypeptide” refers to a wild-type or variant protein (e.g., mutant, fragment, fusion, or combinations thereof). A variant polypeptide may possess at least or about 5%, 10%, 15%, 20%, 30%, 35%, 40%, 45%, 50%, 55%, 60%, 65%, 70%, 75%, 80%, 85%, 90%, 95%, or 100% functional activity of the wild-type polypeptide. In some embodiments, the variant is at least 70%, 75%, 80%, 85%, 90%, 92%, 93%, 94%, 95%, 96%, 97%, 98%, or 99% identical to the sequence of the wild-type polypeptide. In some embodiments, a variant polypeptide may be a hyperactive variant. In certain instances, the variant possesses between about 80% and about 120%, 140%, 160%, 180%, 200%, 300%, 400%, 500%, or more of a functional activity of the wild-type polypeptide.

As used herein, a “heterologous gene” refers to a gene that has been introduced as an exogenous source within a cell (e.g., inserted at a genomic locus such as a safe harbor locus including a TCR gene locus). That is, the introduced gene is heterologous with respect to its insertion site. A polypeptide expressed from such heterologous gene is referred to as a “heterologous polypeptide.” The heterologous gene can be naturally-occurring or engineered, and can be wild-type or a variant. The heterologous gene may include nucleotide sequences other than the sequence that encodes the heterologous polypeptide (e.g., an internal ribosomal entry site). The heterologous gene can be a gene that occurs naturally in the genome, as a wild-type or a variant (e.g., mutant). For example, although the cell contains the gene of interest (as a wild-type or as a variant), the same gene or variant thereof can be introduced as an exogenous source for, e.g., expression at a locus that is highly expressed. The heterologous gene can also be a gene that is not naturally occurring in the genome, or that expresses a heterologous polypeptide that does not naturally occur in the genome. “Heterologous gene”, “exogenous gene”, and “transgene” are used interchangeably. In some embodiments, the heterologous gene or transgene includes an exogenous nucleic acid sequence, e.g., a nucleic acid sequence is not endogenous to the recipient cell. In some embodiments, the heterologous gene or transgene includes an exogenous nucleic acid sequence, e.g., a nucleic acid sequence that does not naturally occur in the recipient cell. For example, a heterologous gene a heterologous gene may be heterologous with respect to its insertion site and with respect to its recipient cell.

A “safe harbor” locus is a locus within the genome wherein a gene may be inserted without significant deleterious effects on the cell. Non-limiting examples of safe harbor loci that are targeted by nuclease(s) for use herein include AAVS1 (PPP1 R12C), TCR, B2M, and any of the loci targeted for knockdown described herein, e.g., TNFA, IFNG, IL17A, and IL6 genomic loci. In some embodiments, insertions at a locus or loci targeted for knockdown such as a TRC gene, e.g., TRAC gene, is advantageous for allogenic cells. Other suitable safe harbor loci are known in the art.

### II. Compositions

### A. Engineered T Cells

Provided herein are T cells and populations of T cells engineered to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under the control of a promoter sequence, as well as compositions and uses thereof. In some embodiments, the regulatory T cell promoting molecule is selected from IL10, CTLA4, TGFB1, IDO1, ENTPD1, NT5E, IL22, AREG, IL35, GARP, CD274, FOXP3, IKZF2, EOS, IRF4, LEF1, and BACH2.

In some embodiments, the T cells or population of T cells is engineered to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding two or more regulatory T cell promoting molecules each under the control of a promoter sequence. For example, the engineered T cell comprises a first heterologous sequence encoding a first regulatory T cell promoting molecule that is under the control of a first promoter and a second heterologous sequence encoding a second regulatory T cell promoting molecule that is under the control of a second promoter. The first promoter and the second promoter may be the same promoter or different promoters.

In some embodiments, the T cells or population of T cells is engineered to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding IL10 that is under the control of a promoter. In some embodiments, the T cell is engineered to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding CTLA4 that is under the control of a promoter. In some embodiments, the T cell is engineered to comprise a modification, e.g., knockdown, of an endogenous nucleic acid sequence encoding an IFNG, a modification, e.g., knockdown, of an endogenous nucleic acid sequence encoding a TNFA, insertion into the cell of heterologous sequence(s) encoding IL10 that is under the control of a promoter, and insertion into the cell of heterologous sequence(s) encoding CTLA4 that is under the control of a promoter.

In some embodiments, the T cells or population of T cells is engineered to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule that is under the control of a promoter, and exhibits at least one suppressive activity of a naturally occurring regulatory T cell (nTreg), e.g., suppression of an immune response or biomarker in an in vitro or in vivo assay, e.g., an animal model of GvHD.

In some embodiments, the heterologous sequence(s) encoding the regulatory T cell promoting molecule is incorporated into an expression construct. In some embodiments, heterologous sequences encoding two or more regulatory T cell promoting molecules may be incorporated into two or more separate expression constructs. For example, a first heterologous sequence encoding a first regulatory T cell promoting molecule is provided in a first expression construct, and a second heterologous sequence encoding a second regulatory T cell promoting molecule is provided in a second, separate expression construct. In some embodiments, the expression construct is an episomal expression construct. In some embodiments, the heterologous sequence(s) encoding the regulatory T cell promoting molecule is inserted into the genome, e.g., a targeted or an untargeted insertion.

In some embodiments, the sequence(s) encoding the regulatory T cell promoting molecule may be inserted into a site selected from a TCR gene locus, e.g., TRAC locus; a TNF gene locus, an IFNG gene locus, a IL17A locus, a IL6 locus, an IL2 locus, or an adeno-associated virus integration site 1 (AAVS1) locus.

In some embodiments, the population of engineered T cell comprises a modification, e.g., knockdown, in a TNFA sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous TNFA sequence. In some embodiments, the expression of TNFA (full-length, wild-type protein or mRNA) is decreased by at least 50%, 55%, 60%, 65%, preferably at least 70%, 75%, 80%, 85%, 90%, 95%, or to below the limit of detection of the assay as compared to a suitable control, e.g., wherein the TNFA gene has not been modified as determined, e.g., by ELISA or flow cytometry. Assays for TNFA protein and mRNA expression, e.g., in the population of T cells, are known in the art and provided herein (see Examples 2 and 3). In certain embodiments, knockdown of TNFA results in a TNFA level of 2500 pg/ml or less by the method provided in Examples 2 and 3.

In some embodiments, the population of engineered T cell comprises a modification, e.g., knockdown, in an IFNG sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous IFNG sequence. In some embodiments, the expression of IFNG (full-length, wild-type protein or mRNA) is decreased by at least 50%, 55%, 60%, 65%, preferably at least 70%, 75%, 80%, 85%, 90%, 95%, or to below the limit of detection of the assay as compared to a suitable control, e.g., wherein the IFNG gene has not been modified as determined, e.g., by ELISA or flow cytometry. Assays for IFNG protein and mRNA expression, e.g., in the population of T cells, are known in the art and provided herein (see Examples 2 and 3). In certain embodiments, knockdown of IFNG results in an IFNG level of 300,000 pg/ml or less by the method provided in Examples 2 and 3.

In some embodiments, the modification that knocks down expression of a gene, e.g., TNFA or IFNG, is one or more of an insertion, a deletion, or a substitution.

In some embodiments, the engineered T cells or population of T cells comprise an insertion of sequence(s) encoding a regulatory T cell promoting molecule, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a regulatory T cell promoting molecule. In some embodiments, the inserted regulatory T cell promoting molecule, e.g., IL10, results in statistically significantly increased expression of protein or mRNA as compared to a suitable control, e.g., wherein the regulatory T cell promoting molecule gene has not been inserted as determined, e.g., by ELISA or flow cytometry. In some embodiments, the engineered T cells comprise an insertion of sequence(s) encoding IL10 by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion or a sequence encoding IL10. In some embodiments, the inserted sequence(s) encoding IL10 results in statistically significantly increased expression of protein or mRNA as compared to a suitable control, e.g., wherein the regulatory T cell promoting molecule. Assays for IL10 protein and mRNA expression, e.g., in the population of T cells, are described herein and known in the art, e.g., ELISA and flow cytometry. In certain embodiments, the level of IL10 is at least 300 pg/ml as determined by the method in Examples 2 and 3.

In some embodiments, the engineered T cells or population of T cells comprises an insertion of sequence(s) encoding CTLA4 e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion or a sequence encoding CTLA4. In some embodiments, the inserted sequence(s) encoding CTLA4 results in statistically significantly increased expression of protein or mRNA as compared to a suitable control, e.g., wherein the regulatory T cell promoting molecule. Assays for CTLA4 protein and mRNA expression, e.g., in the population of T cells, are described herein and known in the art, e.g., ELISA and flow cytometry.

In some embodiments, a population of T cells comprises T cells that are engineered to comprise a modification, e.g., knockdown, of an endogenous nucleic acid sequence encoding an IFNG, a modification, e.g., knockdown, of an endogenous nucleic acid sequence encoding a TNFA, and insertion of sequences encoding a regulatory T cell promoting molecule. In some embodiments, at least 40%, 45%, preferably at least 50%, 55%, 60%, 65%, 70%, 75%, 80%, 85%, 90%, 95%, or 100% (e.g., within the detection limits of the assay used) of the T cells in the population of T cells are engineered to comprise a heterologous regulatory T cell promoting molecule, e.g., as assessed by sequencing, e.g., NGS. In some embodiments, at least 50%, 55%, 60%, 65%, preferably at least 70%, 75%, preferably at least 80%, 85%, 90%, 95%, or 100% of the T cells in the population of T cells are engineered to comprise a modification, e.g., knockdown, of sequence(s) encoding TNFA, e.g., as assessed by sequencing, e.g., NGS. In some embodiments, at least 50%, 55%, 60%, 65%, preferably at least 70%, 75%, 80%, 85%, 90%, 95%, or 100% of the T cells in the population of T cells are engineered to comprise a modification, e.g., knockdown, of sequence(s) encoding IFNG, e.g., as assessed by sequencing, e.g., NGS. In some embodiments, at least 40%, 45%, preferably at least 50%, 55%, 60%, 65%, 70%, 75%, 80%, 85%, 90%, 95%, or 100% of the T cells in the population of T cells are engineered to comprise insertion of sequences encoding a regulatory T cell promoting molecule, e.g., as assessed by sequencing, e.g., NGS. In some embodiments, at least 30%, 35%, preferably at least 40%, 45%, 50%, 55%, 60%, 65%, 70%, 75%, 80%, 85%, 90%, 95% or 100% of the T cells in the population of T cells are engineered to comprise insertion of sequence(s) encoding IL10, e.g., as assessed by sequencing, e.g., NGS. In some embodiments, at least 30%, 35%, preferably at least 40%, 45%, 50%, 55%, 60%, 65%, 70%, 75%, 80%, 85%, 90%, 95%, or 100% of the T cells in the population of T cells are engineered to comprise insertion of sequence(s) encoding CTLA4, e.g., as assessed by sequencing, e.g., NGS.

In some embodiments, the engineered T cells or population of T cells comprising a modification, e.g., knockdown, of an endogenous nucleic acid sequence encoding an IFNG, a modification, e.g., knockdown, of an endogenous nucleic acid sequence encoding a TNFA, and insertion of heterologous sequence(s) encoding a regulatory T cell promoting molecule under control of a promoter sequence, further comprise a modification of an endogenous nucleic acid sequence encoding an interleukin 17A (IL17A), an interleukin 6 (IL6), interleukin 2 (IL2), a perforin 1 (PRF1), a granzyme A (GZMA), or a granzyme B (GZMB), wherein the modification knocks down expression of the IL17A, the IL6, the IL2, the PRF1, the GZMA, or the GZMB, respectively.

In some embodiments, the T cells or population of T cells are engineered using a gene editing system, e.g., using an RNA-guided DNA binding agent. In some embodiments, the T cells are engineered using a CRISPR/Cas gene editing system. In some embodiments, the T cells are engineered using a CRISPR/Cas type II gene editing system, e.g., using Cpf1. In some embodiments, the T cells are engineered using a CRISPR/Cas9 gene editing system, e.g., using SpyCas9. Exemplary Cas9 sequences are provided herein.

In some embodiments, the T cells or population of T cells are engineered using guide RNAs that specifically target sites within the IFNG and TNFA genes to provide knockdown of the of IFNG and TNFA genes. Exemplary sequences are provided in Tables 1 and 2, as are genomic coordinates of the target of each listed guide sequence.

In some embodiments, the engineered T cells or population of T cells comprise IFNG and TNFA genes that are knocked down using a guide RNA disclosed herein with an RNA-guided DNA binding agent. In some embodiments, disclosed herein are T cells engineered by inducing a break (e.g., double-stranded break (DSB) or single-stranded break (nick)) within the IFNG and TNFA genes of a T cell, e.g., using a guide RNA disclosed herein with an RNA-guided DNA-binding agent (e.g., a CRISPR/Cas system). The methods may be used in vitro or ex vivo, e.g., in the manufacture of cell products for suppressing immune response(s), including inflammation and autoimmunity. In some embodiments, the guide RNAs disclosed herein mediate a target-specific cutting by an RNA-guided DNA-binding agent (e.g., Cas nuclease) at a site described herein within an IFNG gene. In some embodiments, the guide RNAs disclosed herein mediate a target-specific cutting by an RNA-guided DNA-binding agent (e.g., Cas nuclease) at a site described herein within a TNFA gene. It will be appreciated that, in some embodiments, the guide RNAs comprise guide sequences that bind to, or are capable of binding to, said regions.

Engineered T cells or population of T cells comprising a genetic modification at genomic coordinates chosen from those listed in Table 1 are provided, e.g., cells comprising an indel or substitution mutation within any of the listed genomic ranges within IFNG. Engineered T cells comprising a genetic modification at genomic coordinates chosen from those listed in Table 2 are also provided, e.g., cells comprising an indel or substitution mutation within any of the listed genomic ranges within TNFA. In some embodiments, the engineered T cell will comprise a modification within a genomic coordinate region chosen from Table 1 and a modification with a genomic coordinate region chosen from Table 2.

In some embodiments, the guide RNAs disclosed herein comprise a guide sequence that is 95%, 90%, 85%, 80%, or 75% identical to a sequence selected from the group of sequences in Table 1 or Table 2. In some embodiments, the guide RNAs disclosed herein comprise a guide sequence that is 95%, 90%, 85%, 80%, or 75% identical to a sequence selected from the group of sequences in Table 1 or Table 2.

In some embodiments, the guide RNAs disclosed herein comprise a guide sequence having at least 15, 16, 17, 18, 19, or 20 contiguous nucleotides of a sequence selected from the group consisting of a sequence that is 95%, 90%, 85%, 80%, or 75% identical to a sequence selected from the group of sequences in Table 1. In some embodiments, the guide RNAs disclosed herein comprise a guide sequence at least 17, 18, 19, or 20 contiguous nucleotides of a sequence selected from the group of sequences in Table 1. In some embodiments, the guide RNAs disclosed herein comprise a guide sequence that is 95%, 90%, 85%, 80%, or 75% identical to a sequence selected from the group of sequences in Table 1. In some embodiments, the guide RNAs disclosed herein comprise a guide sequence that is 17, 18, 19, or 20 contiguous nucleotides of a sequence selected from the group of sequences in Table 1. In some embodiments, the guide RNAs disclosed herein comprise a guide sequence that is selected from the group of sequences in Table 1.

In some embodiments, the guide RNAs disclosed herein comprise a guide sequence having at least 15, 16, 17, 18, 19, or 20 contiguous nucleotides of a sequence selected from the group consisting of a sequence that is 9500, 9000, 85% o, 80% o, or 7500 identical to a sequence selected from the group of sequences in Table 2. In some embodiments, the guide RNAs disclosed herein comprise a guide sequence at least 17, 18, 19, or 20 contiguous nucleotides of a sequence selected from the group of sequences in Table 2. In some embodiments, the guide RNAs disclosed herein comprise a guide sequence that is 95%, 90%, 85%, 80%, or 75% identical to a sequence selected from the group of sequences in Table 2. In some embodiments, the guide RNAs disclosed herein comprise a guide sequence that is 17, 18, 19, or 20 contiguous nucleotides of a sequence selected from the group of sequences in Table 2. In some embodiments, the guide RNAs disclosed herein comprise a guide sequence that is selected from the group of sequences in Table 2.

Genomic coordinates throughout are according to human reference genome hg38 unless otherwise noted.

In certain embodiments a guide RNA comprising a guide sequence targeting IFNG and a guide RNA comprising a guide sequence targeting TNFA are included.

TABLE 1
Human guide sequences and chromosomal coordinates for knockdown of
IFNG

| | | | | SEQ |
|---|---|---|---|---|
| Exemplary Genomic | | | Guide | ID |
| Coordinates (hg38) | Exon | Strand | Sequence (5′ to 3′) | NO |
| chr12:68155336-68155356 | 4 | + | GCAGGCAGGACAACCAUUAC | 1 |
| chr12:68155337-68155357 | 4 | + | CAGGCAGGACAACCAUUACU | 2 |
| chr12:68155374-68155394 | 4 | − | AGGAGUCAGAUGCUGUUUCG | 3 |
| chr12:68155394-68155414 | 4 | − | CUAAAACAGGGAAGCGAAAA | 4 |
| chr12:68155398-68155418 | 4 | + | CGCUUCCCUGUUUUAGCUGC | 5 |
| chr12:68155406-68155426 | 4 | − | UGUCGCCAGCAGCUAAAACA | 6 |
| chr12:68155407-68155427 | 4 | − | CUGUCGCCAGCAGCUAAAAC | 7 |
| chr12:68155420-68155440 | 4 | + | GCGACAGUUCAGCCAUCACU | 8 |
| chr12:68155435-68155455 | 4 | − | ACAUGAACUCAUCCAAGUGA | 9 |
| chr12:68155447-68155467 | 4 | + | GUUCAUGUAUUGCUUUGCGU | 10 |
| chr12:68157972-68157992 | 3 | + | GACAUUCAUGUCUUCCUUGA | 11 |
| chr12:68157989-68158009 | 3 | + | UGAUGGUCUCCACACUCUUU | 12 |
| chr12:68157989-68158009 | 3 | − | AAAGAGUGUGGAGACCAUCA | 13 |
| chr12:68158001-68158021 | 3 | − | CCAGAGCAUCCAAAAGAGUG | 14 |
| chr12:68158215-68158235 | 2 | − | GAUAAUGGAACUCUUUUCUU | 15 |
| chr12:68158230-68158250 | 2 | − | CAUUCAGAUGUAGCGGAUAA | 16 |
| chr12:68158237-68158257 | 2 | − | UGCAGGUCAUUCAGAUGUAG | 17 |
| chr12:68159524-68159544 | 1 | + | CUUCUUUUACAUAUGGGUCC | 18 |
| chr12:68159559-68159579 | 1 | − | UGCAUCGUUUUGGGUUCUCU | 19 |
| chr12:68159568-68159588 | 1 | − | UUUCAGCUCUGCAUCGUUUU | 20 |
| chr12:68159569-68159589 | 1 | − | UUUUCAGCUCUGCAUCGUUU | 21 |

TABLE 2
Human guide sequences and chromosomal coordinates for knockdown of
TNFA

| | | | | SEQ |
|---|---|---|---|---|
| Exemplary Genomic | | | Guide | ID |
| Coordinates (hg38) | Exon | Strand | sequence (5′ to 3′) | NO |
| chr6:31575742-31575762 | 1 | + | UGAGCACUGAAAGCAUGAUC | 22 |
| chr6:31575749-31575769 | 1 | + | UGAAAGCAUGAUCCGGGACG | 23 |
| chr6:31575755-31575775 | 1 | + | CAUGAUCCGGGACGUGGAGC | 24 |
| chr6:31575781-31575801 | 1 | − | CUUCUUGGGGAGCGCCUCCU | 25 |
| chr6:31575783-31575803 | 1 | + | GAGGCGCUCCCCAAGAAGAC | 26 |
| chr6:31575784-31575804 | 1 | + | AGGCGCUCCCCAAGAAGACA | 27 |
| chr6:31575785-31575805 | 1 | + | GGCGCUCCCCAAGAAGACAG | 28 |
| chr6:31575786-31575806 | 1 | + | GCGCUCCCCAAGAAGACAGG | 29 |
| chr6:31575794-31575814 | 1 | + | CAAGAAGACAGGGGGGCCCC | 30 |
| chr6:31575794-31575814 | 1 | − | GGGGCCCCCCUGUCUUCUUG | 31 |
| chr6:31575796-31575816 | 1 | − | CUGGGGCCCCCCUGUCUUCU | 32 |
| chr6:31575813-31575833 | 1 | − | AAGCACCGCCUGGAGCCCUG | 33 |
| chr6:31575814-31575834 | 1 | − | CAAGCACCGCCUGGAGCCCU | 34 |
| chr6:31575823-31575843 | 1 | − | GCUGAGGAACAAGCACCGCC | 35 |
| chr6:31575842-31575862 | 1 | + | CCUCUUCUCCUUCCUGAUCG | 36 |
| chr6:31575846-31575866 | 1 | + | UUCUCCUUCCUGAUCGUGGC | 37 |
| chr6:31575853-31575873 | 1 | − | GGCGCCUGCCACGAUCAGGA | 38 |
| chr6:31575857-31575877 | 1 | − | UGGUGGCGCCUGCCACGAUC | 39 |
| chr6:31575888-31575908 | 1 | + | CUGCUGCACUUUGGAGUGAU | 40 |
| chr6:31575890-31575910 | 1 | − | CGAUCACUCCAAAGUGCAGC | 41 |
| chr6:31575898-31575918 | 1 | + | UUGGAGUGAUCGGCCCCCAG | 42 |
| chr6:31575899-31575919 | 1 | + | UGGAGUGAUCGGCCCCCAGA | 43 |
| chr6:31575917-31575937 | 1 | − | AGGCACUCACCUCUUCCCUC | 44 |
| chr6:31576538-31576558 | 2 | − | UGAUUAGAGAGAGGUCCCUG | 45 |
| chr6:31576539-31576559 | 2 | − | CUGAUUAGAGAGAGGUCCCU | 46 |
| chr6:31576540-31576560 | 2 | − | GCUGAUUAGAGAGAGGUCCC | 47 |
| chr6:31576544-31576564 | 2 | + | CCUCUCUCUAAUCAGCCCUC | 48 |
| chr6:31576547-31576567 | 2 | − | CCAGAGGGCUGAUUAGAGAG | 49 |
| chr6:31576550-31576570 | 2 | + | UCUAAUCAGCCCUCUGGCCC | 50 |
| chr6:31576562-31576582 | 2 | − | UACUGACUGCCUGGGCCAGA | 51 |
| chr6:31576563-31576583 | 2 | − | UUACUGACUGCCUGGGCCAG | 52 |
| chr6:31576570-31576590 | 2 | − | GAGACACUUACUGACUGCCU | 53 |
| chr6:31576571-31576591 | 2 | − | GGAGACACUUACUGACUGCC | 54 |
| chr6:31576785-31576805 | 3 | − | GGGCUACAGGCUUGUCACUC | 55 |
| chr6:31576786-31576806 | 3 | − | UGGGCUACAGGCUUGUCACU | 56 |
| chr6:31576798-31576818 | 3 | − | UUACCUACAACAUGGGCUAC | 57 |
| chr6:31576805-31576825 | 3 | − | AGAGCUCUUACCUACAACAU | 58 |
| chr6:31576806-31576826 | 3 | − | CAGAGCUCUUACCUACAACA | 59 |
| chr6:31577110-31577130 | 4 | + | UCCAGCAAACCCUCAAGCUG | 60 |
| chr6:31577112-31577132 | 4 | + | CAGCAAACCCUCAAGCUGAG | 61 |
| chr6:31577122-31577142 | 4 | − | GGAGCUGCCCCUCAGCUUGA | 62 |
| chr6:31577123-31577143 | 4 | − | UGGAGCUGCCCCUCAGCUUG | 63 |
| chr6:31577136-31577156 | 4 | + | AGCUCCAGUGGCUGAACCGC | 64 |
| chr6:31577137-31577157 | 4 | + | GCUCCAGUGGCUGAACCGCC | 65 |
| chr6:31577143-31577163 | 4 | − | UGGCCCGGCGGUUCAGCCAC | 66 |
| chr6:31577152-31577172 | 4 | + | CCGCCGGGCCAAUGCCCUCC | 67 |
| chr6:31577155-31577175 | 4 | − | CCAGGAGGGCAUUGGCCCGG | 68 |
| chr6:31577159-31577179 | 4 | + | GCCAAUGCCCUCCUGGCCAA | 69 |
| chr6:31577163-31577183 | 4 | − | GCCAUUGGCCAGGAGGGCAU | 70 |
| chr6:31577164-31577184 | 4 | + | UGCCCUCCUGGCCAAUGGCG | 71 |
| chr6:31577170-31577190 | 4 | − | GCUCCACGCCAUUGGCCAGG | 72 |
| chr6:31577173-31577193 | 4 | − | UCAGCUCCACGCCAUUGGCC | 73 |
| chr6:31577178-31577198 | 4 | − | AUCUCUCAGCUCCACGCCAU | 74 |
| chr6:31577185-31577205 | 4 | + | GGAGCUGAGAGAUAACCAGC | 75 |
| chr6:31577188-31577208 | 4 | + | GCUGAGAGAUAACCAGCUGG | 76 |
| chr6:31577200-31577220 | 4 | + | CCAGCUGGUGGUGCCAUCAG | 77 |
| chr6:31577201-31577221 | 4 | + | CAGCUGGUGGUGCCAUCAGA | 78 |
| chr6:31577216-31577236 | 4 | − | AUGAGGUACAGGCCCUCUGA | 79 |
| chr6:31577227-31577247 | 4 | − | CCUGGGAGUAGAUGAGGUAC | 80 |
| chr6:31577237-31577257 | 4 | + | UACUCCCAGGUCCUCUUCAA | 81 |
| chr6:31577245-31577265 | 4 | − | CUUGGCCCUUGAAGAGGACC | 82 |
| chr6:31577295-31577315 | 4 | − | GGCGAUGCGGCUGAUGGUGU | 83 |
| chr6:31577296-31577316 | 4 | − | CGGCGAUGCGGCUGAUGGUG | 84 |
| chr6:31577301-31577321 | 4 | − | GGAGACGGCGAUGCGGCUGA | 85 |
| chr6:31577308-31577328 | 4 | − | UCUGGUAGGAGACGGCGAUG | 86 |
| chr6:31577311-31577331 | 4 | + | CGCCGUCUCCUACCAGACCA | 87 |
| chr6:31577322-31577342 | 4 | − | GAGGUUGACCUUGGUCUGGU | 88 |
| chr6:31577331-31577351 | 4 | − | GGCAGAGAGGAGGUUGACCU | 89 |
| chr6:31577349-31577369 | 4 | + | CCAUCAAGAGCCCCUGCCAG | 90 |
| chr6:31577350-31577370 | 4 | + | CAUCAAGAGCCCCUGCCAGA | 91 |
| chr6:31577362-31577382 | 4 | − | CUGGGGUCUCCCUCUGGCAG | 92 |
| chr6:31577363-31577383 | 4 | − | UCUGGGGUCUCCCUCUGGCA | 93 |
| chr6:31577397-31577417 | 4 | − | GAUGGGCUCAUACCAGGGCU | 94 |
| chr6:31577401-31577421 | 4 | + | CUGGUAUGAGCCCAUCUAUC | 95 |
| chr6:31577402-31577422 | 4 | − | AGAUAGAUGGGCUCAUACCA | 96 |
| chr6:31577402-31577422 | 4 | + | UGGUAUGAGCCCAUCUAUCU | 97 |
| chr6:31577403-31577423 | 4 | − | CAGAUAGAUGGGCUCAUACC | 98 |
| chr6:31577406-31577426 | 4 | + | AUGAGCCCAUCUAUCUGGGA | 99 |
| chr6:31577407-31577427 | 4 | + | UGAGCCCAUCUAUCUGGGAG | 100 |
| chr6:31577414-31577434 | 4 | − | AAGACCCCUCCCAGAUAGAU | 101 |
| chr6:31577437-31577457 | 4 | − | GUCGGUCACCCUUCUCCAGC | 102 |
| chr6:31577454-31577474 | 4 | + | GACUCAGCGCUGAGAUCAAU | 103 |
| chr6:31577455-31577475 | 4 | − | GAUUGAUCUCAGCGCUGAGU | 104 |
| chr6:31577480-31577500 | 4 | − | UCGGCAAAGUCGAGAUAGUC | 105 |
| chr6:31577481-31577501 | 4 | − | CUCGGCAAAGUCGAGAUAGU | 106 |
| chr6:31577483-31577503 | 4 | + | UAUCUCGACUUUGCCGAGUC | 107 |
| chr6:31577484-31577504 | 4 | + | AUCUCGACUUUGCCGAGUCU | 108 |
| chr6:31577488-31577508 | 4 | + | CGACUUUGCCGAGUCUGGGC | 109 |
| chr6:31577498-31577518 | 4 | + | GAGUCUGGGCAGGUCUACUU | 110 |
| chr6:31577499-31577519 | 4 | + | AGUCUGGGCAGGUCUACUUU | 111 |
| chr6:31577499-31577519 | 4 | − | AAAGUAGACCUGCCCAGACU | 112 |
| chr6:31577532-31577552 | 4 | − | GGAUGUUCGUCCUCCUCACA | 113 |

A non-limiting modified guide sequence for knockdown of TNFA is shown below (hg38 coordinates chr12:68158001-68158021): mC*mC*mA*GAGCAUCCAAAAGAGUGGUUUUJIAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ TD NO: 119), wherein m is a 2′-OMe modified nucleotide/nucleoside residue, * is indicative of a phosphorothioate linkage between the residues, a capital letter indicates a residue, preferably comprising a ribose sugar.

A non-limiting modified guide sequence for knockdown of IFNG is shown below (hg38 coordinates chr6:31576805-31576825):

(SEQ ID NO: 120)
mA*mG*mA*GCUCUUACCUACAACAUGUUUUAGAmGmCmUmAmGmAmAm
AmUmAmGmCAAGUUAAAAUAAGGCUAGUCCGUUAUCAmAmCmUmUmGmA
mAmAmAmAmGmUmGmGmCmAmCmCmGmAmGmUmCmGmGmUmGmCmU*mU
*mU*mU

An exemplary modified mock guide is shown below (hg38 coordinates chr1:0-20):

(SEQ ID NO: 121)
mG*mA*mU*CACGUCGGCCGUUGGCGGUUUUAGAmGmCmUmAmGmAmAm
AmUmAmGmCAAGUUAAAAUAAGGCUAGUCCGUUAUCAmAmCmUmUmGmA
mAmAmAmAmGmUmGmGmCmAmCmCmGmAmGmUmCmGmGmUmGmCmU*mU
*mU*mU.

In some embodiments, disclosed herein are T cells engineered by introducing or inserting a heterologous IL10 nucleic acid within a genomic locus of a T cell or a population of T cells using a guide RNA with an RNA-guided DNA binding agent, and a construct (e.g., donor construct or template) comprising a heterologous IL10 nucleic acid, e.g., to make an engineered T cell. In some embodiments, disclosed herein are T cells engineered by expressing a heterologous IL10 from a genomic locus of a T cell or a population of T cells, e.g., using a guide RNA with an RNA-guided DNA-binding agent and a construct (e.g., donor) comprising a heterologous IL10 nucleic acid. In some embodiments, disclosed herein are T cells engineered by inducing a break (e.g., double-stranded break (DSB) or single-stranded break (nick)) within the genome of a T cell or a population of T cells for inserting the IL10 gene, e.g., using a guide RNA with an RNA-guided DNA-binding agent (e.g., a CRISPR/Cas system). Cells and cell populations made by the methods are also provided.

In some embodiments, disclosed herein are T cells engineered by introducing or inserting a heterologous CTLA4 nucleic acid within a genomic locus of a T cell or a population of T cells using a guide RNA with an RNA-guided DNA binding agent, and a construct (e.g., donor construct or template) comprising a heterologous CTLA4 nucleic acid, e.g., to make an engineered T cell. In some embodiments, disclosed herein are T cells engineered by expressing a heterologous CTLA4 from the genomic locus of a T cell or a population of T cells, e.g., using a guide RNA with an RNA-guided DNA-binding agent and a construct (e.g., donor) comprising a heterologous CTLA4 nucleic acid. In some embodiments, disclosed herein are T cells engineered by inducing a break (e.g., double-stranded break (DSB) or single-stranded break (nick)) within the genome of a T cell or a population of T cells for inserting the CTLA4 gene, e.g., using a guide RNA with an RNA-guided DNA-binding agent (e.g., a CRISPR/Cas system). Cells and cell populations made by the methods are also provided.

In some embodiments, disclosed herein are T cells engineered by introducing or inserting a heterologous CTLA4 nucleic acid and a heterologous IL10 nucleic acid within a genomic locus of a T cell or a population of T cells using a guide RNA with an RNA-guided DNA binding agent, and one or more constructs (e.g., donor construct or template) comprising a heterologous CTLA4 nucleic acid and a heterologous IL10 nucleic acid, e.g., to make an engineered T cell. In some embodiments, disclosed herein are T cells engineered by expressing a heterologous CTLA4 and a heterologous IL10 from the genomic locus of a T cell or a population of T cells, e.g., using a guide RNA with an RNA-guided DNA-binding agent and one or more constructs (e.g., donor construct or template) comprising a heterologous CTLA4 nucleic acid and a heterologous IL10 nucleic acid. In some embodiments, disclosed herein are T cells engineered by inducing a break (e.g., double-stranded break (DSB) or single-stranded break (nick)) within the genome of a T cell or a population of T cells for inserting the CTLA4 gene and the IL10 gene, e.g., using a guide RNA with an RNA-guided DNA-binding agent (e.g., a CRISPR/Cas system). In some embodiments, the guide RNAs mediate a target-specific cutting by an RNA-guided DNA-binding agent (e.g., Cas nuclease) at a site described herein for insertion of sequence(s) encoding two or more regulatory T cell promoting molecule, e.g., IL10 and CTLA4. It will be appreciated that, in some embodiments, the guide RNAs comprise guide sequences that bind to, or are capable of binding to, said regions. Cells and cell populations made by the methods are also provided.

Exemplary nucleotide and polypeptide sequences of regulatory T cell promoting molecules are provided below. Methods for identifying alternate nucleotide sequences encoding polypeptide sequences, including alternate naturally occurring variants and non-human homologues, are known in the art. Exemplary nucleic acid sequences encoding IL10 and CTLA4 are provided below. Other suitable IL10 and CTLA4 sequences are known in the art. See, e.g., Gorby et al., Engineered IL-10 variants elicit potent immuno-modulatory activities at therapeutic low ligand doses, BioRxiv (2020) and Xu et al., Affinity and cross-reactivity engineering of CTLA4-Ig to modulate T cell costimulation, J Immunol (2012), the contents and sequences of which are hereby incorporated by reference. Methods for identifying alternate IL10 and CTLA4 sequences are also known in the art. See, e.g., id. Sequences with at least 75%, 80%, 85%, 90%, 95%, 96%, 97%, 98%, or 99% identity to any of the nucleic acid sequences, amino acid sequences, or nucleic acid sequences encoding the amino acid sequences described herein, e.g., due to mutations or truncations, are also contemplated. In some embodiments, a nucleic acid sequence encoding any of the amino acid sequences provided herein is also provided.

Non-limiting exemplary nucleic acid sequences encoding IL10 are provided:

Wild-type IL10:
(SEQ ID NO: 122)
ATGCACAGCTCAGCACTGCTCTGTTGCCTGGTCCTCCTGACTGGGGTGA
GGGCCAGCCCAGGCCAGGGCACCCAGTCTGAGAACAGCTGCACCCACTT
CCCAGGCAACCTGCCTAACATGCTTCGAGATCTCCGAGATGCCTTCAGC
AGAGTGAAGACTTTCTTTCAAATGAAGGATCAGCTGGACAACTTGTTGT
TAAAGGAGTCCTTGCTGGAGGACTTTAAGGGTTACCTGGGTTGCCAAGC
CTTGTCTGAGATGATCCAGTTTTACCTGGAGGAGGTGATGCCCCAAGCT
GAGAACCAAGACCCAGACATCAAGGCGCATGTGAACTCCCTGGGGGAGA
ACCTGAAGACCCTCAGGCTGAGGCTACGGCGCTGTCATCGATTTCTTCC
CTGTGAAAACAAGAGCAAGGCCGTGGAGCAGGTGAAGAATGCCTTTAAT
AAGCTCCAAGAGAAAGGCATCTACAAAGCCATGAGTGAGTTTGACATCT
TCATCAACTACATAGAAGCCTACATGACAATGAAGATACGAAAC
High affinity IL10 (N36I, N110I, K117N, F129L):
(SEQ ID NO: 123)
ATGCACAGCTCAGCACTGCTCTGTTGCCTGGTCCTCCTGACTGGGGTGA
GGGCCAGCCCAGGCCAGGGCACCCAGTCTGAGAACAGCTGCACCCACTT
CCCAGGCATCCTGCCTAACATGCTTCGAGATCTCCGAGATGCCTTCAGC
AGAGTGAAGACTTTCTTTCAAATGAAGGATCAGCTGGACAACTTGTTGT
TAAAGGAGTCCTTGCTGGAGGACTTTAAGGGTTACCTGGGTTGCCAAGC
CTTGTCTGAGATGATCCAGTTTTACCTGGAGGAGGTGATGCCCCAAGCT
GAGAACCAAGACCCAGACATCAAGGCGCATGTGatcTCCCTGGGGGAGA
ACCTGAATACCCTCAGGCTGAGGCTACGGCGCTGTCATCGActcCTTCC
CTGTGAAAACAAGAGCAAGGCCGTGGAGCAGGTGAAGAATGCCTTTAAT
AAGCTCCAAGAGAAAGGCATCTACAAAGCCATGAGTGAGTTTGACATCT
TCATCAACTACATAGAAGCCTACATGACAATGAAGATACGAAAC

Non-limiting exemplary amino acid sequences of IL10 are provided:

Wild-type IL10:
(SEQ ID NO: 124)
MHSSALLCCLVLLTGVRASPGQGTQSENSCTHFPGNLPNMLRDLRDAFS
RVKTFFQMKDQLDNLLLKESLLEDFKGYLGCQALSEMIQFYLEEVMPQA
ENQDPDIKAHVNSLGENLKTLRLRLRRCHRFLPCENKSKAVEQVKNAFN
KLQEKGIYKAMSEFDIFINYIEAYMTMKIRN
High affinity IL10 (N36I, N110I, K117N, F129L):
(SEQ ID NO: 125)
MHSSALLCCLVLLTGVRASPGQGTQSENSCTHFPGILPNMLRDLRDAFS
RVKTFFQMKDQLDNLLLKESLLEDFKGYLGCQALSEMIQFYLEEVMPQA
ENQDPDIKAHVISLGENLNTLRLRLRRCHRLLPCENKSKAVEQVKNAFN
KLQEKGIYKAMSEFDIFINYIEAYMTMKIRN

Non-limiting exemplary nucleic acid sequences encoding CTLA4 are provided:

Wild-type CTLA4:
(SEQ ID NO: 126)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAAGCGACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAAC
High affinity CTLA4 (belatacept; Binding domain: A29Y, L104E):
(SEQ ID NO: 127)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAATACACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATGAGGGGATCGGAAATGGGA
CACAGATATATGTTATTGATCCCGAGCCATGTCCCGATAGTGACTTCCTCTTGTG
GATACTTGCCGCTGTGAGCAGTGGTTTGTTTTTTTATTCATTCCTCCTTACGGCAG
TATCACTTTCAAAAATGCTCAAGAAGCGAAGTCCTTTGACAACTGGCGTATATGT
CAAAATGCCACCAACAGAGCCCGAATGTGAGAAACAGTTCCAGCCGTACTTTAT
TCCTATAAAC
High affinity CTLA4 (Binding domain: A29H):
(SEQ ID NO: 128)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAACATACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAAC
High affinity CTLA4 (Binding domain: K28H, A29H):
(SEQ ID NO: 129)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGACATCACACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAAC

Non-limiting exemplary amino acid sequences of CTLA4 are provided:

Wild-type CTLA4:
(SEQ ID NO: 130)
MACLGFQRHKAQLNLATRTWPCTLLFFLLFIPVFCKAMHVAQPAVVLAS
SRGIASFVCEYASPGKATEVRVTVLRQADSQVTEVCAATYMMGNELTFL
DDSICTGTSSGNQVNLTIQGLRAMDTGLYICKVELMYPPPYYLGIGNGT
QIYVIDPEPCPDSDFLLWILAAVSSGLFFYSFLLTAVSLSKMLKKRSPL
TTGVYVKMPPTEPECEKQFQPYFIPIN
High affinity CTLA4 (belatacept; Binding domain:
A29Y, L104E):
(SEQ ID NO: 131)
MACLGFQRHKAQLNLATRTWPCTLLFFLLFIPVFCKAMHVAQPAVVLAS
SRGIASFVCEYASPGKYTEVRVTVLRQADSQVTEVCAATYMMGNELTFL
DDSICTGTSSGNQVNLTIQGLRAMDTGLYICKVELMYPPPYYEGIGNGT
QIYVIDPEPCPDSDFLLWILAAVSSGLFFYSFLLTAVSLSKMLKKRSPL
TTGVYVKMPPTEPECEKQFQPYFIPIN
High affinity CTLA4 (Binding domain: A29H):
(SEQ ID NO: 132)
MACLGFQRHKAQLNLATRTWPCTLLFFLLFIPVFCKAMHVAQPAVVLAS
SRGIASFVCEYASPGKHTEVRVTVLRQADSQVTEVCAATYMMGNELTFL
DDSICTGTSSGNQVNLTIQGLRAMDTGLYICKVELMYPPPYYLGIGNGT
QIYVIDPEPCPDSDFLLWILAAVSSGLFFYSFLLTAVSLSKMLKKRSPL
TTGVYVKMPPTEPECEKQFQPYFIPIN
High affinity CTLA4 (Binding domain: K28H, A29H):
(SEQ ID NO: 133)
MACLGFQRHKAQLNLATRTWPCTLLFFLLFIPVFCKAMHVAQPAVVLAS
SRGIASFVCEYASPGHHTEVRVTVLRQADSQVTEVCAATYMMGNELTFL
DDSICTGTSSGNQVNLTIQGLRAMDTGLYICKVELMYPPPYYLGIGNGT
QIYVIDPEPCPDSDFLLWILAAVSSGLFFYSFLLTAVSLSKMLKKRSPL
TTGVYVKMPPTEPECEKQFQPYFIPIN

In some embodiments, the engineered T cells or population of T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, exhibits at least one suppressive activity of a naturally occurring regulatory T cell (nTreg), e.g., suppression of an immune response(s) or biomarker in an in vitro or in vivo assay, e.g., an animal model of GvHD. In some embodiments, the engineered T cells or population of T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, exhibits improved suppressive activity as compared to a nTreg, e.g., increased suppression of an immune response or biomarker in an in vitro or in vivo assay, e.g., an animal model of GvHD. For example, in a mouse model of GvHD, mice receiving the engineered T cell comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, exhibit improved survival compared to a control, e.g., mice receiving PBMC.

### B. Targeting Receptor

In some embodiments, the engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, further comprise insertion into the cell of heterologous sequence(s) encoding a targeting receptor. The sequence(s) encoding the targeting receptor is under the control of a promoter sequence, e.g., an endogenous promoter or a heterologous promoter.

In some embodiments, the targeting receptor is a chimeric antigen receptor (CAR), a T-cell receptor (TCR), or a receptor for a cell surface molecule operably linked through at least a transmembrane domain in an internal signaling domain capable of activating a T cell upon binding of the extracellular receptor portion. In some embodiments, the targeting receptor may be a receptor present on the surface of a cell, e.g., a T cell, to permit binding of the cell to a target site, e.g., a specific cell or tissue in an organism. The targeting receptor need not be an antigen receptor, e.g., the targeting receptor may be an RGD peptide that is capable of targeting an integrin. In some embodiments, the targeting receptor targets a molecule selected from the group consisting of MAdCAM-1, TNFA, CEACAM6, VCAM-1, citrullinated vimentin, myelin basic protein (MBP), MOG (myelin oligodendrocyte glycoprotein), proteolipid protein 1 (PLP1), CD19 molecule (CD19), CD20 molecule (CD20), TNFRSF17, dipeptidyl peptidase like 6 (DPP6), solute carrier family 2 member 2 (SCL2A2), glutamate decarboxylase (GAD2), demoglein 3 (DSG3), and MHC class I HLA-A (HLA-A*02).

In some embodiments, the engineered T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule selected from IL10, CTLA4, TGFB1, IDO1, ENTPD1, NT5E, IL22, AREG, IL35, GARP, CD274, FOXP3, IKZF2, EOS, IRF4, LEF1, and BACH2, and insertion of sequence(s) encoding a targeting receptor, e.g., a CAR, e.g., a CAR targeting MAdCAM-1.

In some embodiments, the engineered T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding IL10, and insertion of sequence(s) encoding a targeting receptor, e.g., a CAR, e.g., a CAR targeting MAdCAM-1.

In some embodiments, the engineered T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding CTLA4, and insertion of sequence(s) encoding a targeting receptor, e.g., a CAR, e.g., a CAR targeting MAdCAM-1.

In some embodiments, the engineered T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding IL10, insertion of sequence(s) encoding CTLA4, and insertion of sequence(s) encoding a targeting receptor, e.g., a CAR, e.g., a CAR targeting MAdCAM-1.

In some embodiments, the sequence(s) encoding the targeting receptor is incorporated into an expression construct. In some embodiments, the expression construct comprising the sequence(s) encoding the targeting receptor further comprises sequence(s) encoding a regulatory T cell promoting molecule, e.g., the sequence(s) encoding the targeting receptor and the sequence(s) encoding the regulatory T cell promoting molecule are incorporated into the same expression construct. In some embodiments, the expression construct comprising the sequence(s) encoding the targeting receptor does not further comprise sequence(s) encoding a regulatory T cell promoting molecule, e.g., the sequence(s) encoding the regulatory T cell promoting molecule are incorporated into a separate expression construct. In some embodiments, the expression construct comprising the sequence(s) encoding the targeting receptor is an episomal expression construct. In some embodiments, the sequence(s) encoding the targeting receptor is inserted into the genome, e.g., a targeted or an untargeted insertion.

In some embodiments, the sequence(s) encoding the targeting receptor may be inserted into a site selected from a TCR gene locus, e.g., TRAC locus, a TNF gene locus, an IFNG gene locus, IL17A locus, a IL6 locus, an IL2 locus, or an adeno-associated virus integration site 1 (AAVS1) locus.

In some embodiments, the engineered T cells comprise an insertion of sequence(s) encoding a targeting receptor by gene editing, e.g., as assessed by sequencing, e.g., NGS. In some embodiments, a population of T cells comprises T cells that are engineered to comprise a modification, e.g., knockdown, of an endogenous nucleic acid sequence encoding an IFNG, a modification, e.g., knockdown, of an endogenous nucleic acid sequence encoding a TNFA, insertion of sequences encoding a regulatory T cell promoting molecule, and insertion of sequence(s) encoding a targeting receptor, e.g., a CAR. In some embodiments, at least 40%, 45%, preferably at least 50%, 55%, 60%, 65%, 70%, 75%, 80%, 85%, 90%, 95%, or 100% of the T cells in the population of T cells are engineered to comprise insertion of sequence(s) encoding the targeting receptor, e.g., as assessed by sequencing, e.g., NGS. It is understood that a T cell population can be enriched for a population of cells having a targeting receptor using selection methods known in the art.

In some embodiments, disclosed herein are T cells engineered by introducing or inserting a targeting receptor, e.g., a CAR, nucleic acid within a T cell, e.g., within a genomic locus of a T cell or a population of T cells using a guide RNA with an RNA-guided DNA binding agent, and a construct (e.g., donor construct or template) comprising a targeting receptor, e.g., a CAR, nucleic acid, e.g., to make an engineered T cell. In some embodiments, disclosed herein are T cells engineered by expressing a targeting receptor, e.g., a CAR, from the genomic locus of a T cell or a population of T cells, e.g., using a guide RNA with an RNA-guided DNA-binding agent and a construct (e.g., donor) comprising a targeting receptor, e.g., a CAR, nucleic acid. In some embodiments, disclosed herein are T cells engineered by inducing a break (e.g., double-stranded break (DSB) or single-stranded break (nick)) within the genome of a T cell or a population of T cells for inserting the targeting receptor, e.g., a CAR, e.g., using a guide RNA with an RNA-guided DNA-binding agent (e.g., a CRISPR/Cas system). Cells and cell populations made by the methods are also provided.

In some embodiments, the targeting receptor, e.g., a CAR, is capable of conferring target specificity to the engineered T cell comprising the targeting receptor, e.g., a CAR, e.g., to particular cells, tissues, or organs.

In some embodiments, the targeting receptor, e.g., a CAR, is capable of targeting engineered T cells to the gastrointestinal system, e.g., the targeting receptor is a CAR targeting MAdCAM-1, e.g., for suppressing immune responses in disorders such as inflammatory bowel disease, ulcerative colitis, or Crohn's disease.

In some embodiments, the targeting receptor, e.g., a CAR, is capable of targeting engineered T cells to an inflammatory tissue, e.g., the targeting receptor is a CAR targeting TNFA, e.g., for suppressing immune responses in disorders such as inflammatory bowel disease, ulcerative colitis, or Crohn's disease.

In some embodiments, the targeting receptor, e.g., a CAR, is capable of targeting engineered T cells to endothelial cells, e.g., the targeting receptor is a CAR targeting CEACAM6, e.g., for suppressing immune response(s), including inflammation, in disorders such as Crohn's disease.

In some embodiments, the targeting receptor, e.g., a CAR, is capable of targeting engineered T cells to tissues comprising endothelial cells, e.g., the targeting receptor is a CAR targeting VCAM-1, e.g., for suppressing immune responses in disorders such as Crohn's disease and multiple sclerosis.

In some embodiments, the CAR is capable of targeting engineered T cells to synovial tissue, e.g., the targeting receptor is a CAR targeting citrullinated vimentin e.g., for suppressing immune responses in disorders such as rheumatoid arthritis.

In some embodiments, the targeting receptor, e.g., a CAR, is capable of targeting engineered T cells to a neurological tissue, e.g., the targeting receptor is a CAR targeting MBP, MOG, or PLP1, e.g., for suppressing immune responses in disorders such as multiple sclerosis.

In some embodiments, the targeting receptor, e.g., a CAR, is capable of targeting engineered T cells to B cells, e.g., the targeting receptor is a CAR targeting CD19, e.g., for suppressing immune responses in disorders such as multiple sclerosis and systemic lupus erythematosus.

In some embodiments, the targeting receptor, e.g., a CAR, is capable of targeting engineered T cells to B cells, e.g., the targeting receptor is a CAR targeting CD20, e.g., for suppressing immune responses in disorders such as multiple sclerosis and systemic lupus erythematosus.

In some embodiments, the targeting receptor, e.g., a CAR, is capable of targeting engineered T cells to tissues comprising mature B lymphocytes, e.g., the targeting receptor is a CAR targeting TNFRSF17, e.g., for suppressing immune responses in disorders such as systemic lupus erythematosus.

In some embodiments, the targeting receptor, e.g., a CAR, targets SCL2A2. In some embodiments, the targeting receptor, e.g., a CAR, targets DPP6. In some embodiments, the targeting receptor, e.g., a CAR, targets GAD2. In some embodiments, the targeting receptor, e.g., a CAR, targets DSG3. In some embodiments, the targeting receptor, e.g., a CAR, targets NMC class I HLA-A (HLA-A*02).

Additional CAR targets, e.g., inflammatory antigens, are known in the art. See, e.g., WO2020092057A1, the contents of which are incorporated herein in their entirety. In some embodiments, the insertion can be assessed by detecting the amount of protein or mRNA in an engineered T cell, population of engineered T cells, tissue, body fluid of interest, or tissue culture media comprising the engineered T cells. In some embodiments, the insertion by gene editing can be assessed by sequence, e.g., next generation sequencing (NGS). Assays for protein and mRNA expression of the targeting receptor, e.g., a CAR, are described herein and known in the art.

In some embodiments, the engineered T cells or population of T cells do not include a heterologous targeting receptor.

### C. T Cell Receptor (TCR)

In some embodiments, the engineered T cells or population of T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, further comprise a modification, e.g., knockdown, of an endogenous nucleic acid sequence encoding TCR gene sequence(s).

In some embodiments, the engineered T cells or population of T cells comprising a modification, e.g., knockdown, of an endogenous nucleic acid sequence encoding an IFNG, a modification, e.g., knockdown, of an endogenous nucleic acid sequence encoding a TNFA, insertion into the cell of heterologous sequence(s) encoding a regulatory T cell promoting molecule under control of a promoter sequence, insertion into the cell of heterologous sequence(s) encoding a targeting receptor, further comprise a modification, e.g., knockdown, of an endogenous nucleic acid sequence encoding TCR gene sequence(s).

Generally, a TCR is a heterodimer receptor molecule that contains two TCR polypeptide chains, a and p. Suitable a and p genomic sequences or loci to target for knockdown are known in the art. In some embodiments, the engineered T cells comprise a modification, e.g., knockdown, of a TCR α-chain gene sequence, e.g., TRAC. See, e.g., NCBI Gene ID: 28755; Ensembl: ENSG00000277734 (T-cell receptor Alpha Constant), US 2018/0362975, and WO2020081613.

In some embodiments, the engineered T cells or population of T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule selected from IL10, CTLA4, TGFB1, IDO1, ENTPD1, NT5E, IL22, AREG, IL35, GARP, CD274, FOXP3, IKZF2, EOS, IRF4, LEF1, and BACH2, and a modification, e.g., knockdown, of an endogenous nucleic acid sequence encoding TCR gene sequence(s).

In some embodiments, the engineered T cells or population of T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding IL10 or CTLA4, and a modification, e.g., knockdown, of an endogenous nucleic acid sequence encoding TCR gene sequence(s).

In some embodiments, the engineered T cells or population of T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule, and a modification, e.g., knockdown, of an endogenous TCR gene sequence, e.g., TRAC gene sequence.

In some embodiments, the engineered T cells or population of T cells comprise a modification, e.g., knockdown, of an endogenous nucleic acid sequence encoding an IFNG, a modification, e.g., knockdown, of an endogenous nucleic acid sequence encoding a TNFA,
insertion of sequence(s) encoding a regulatory T cell promoting molecule selected from
   IL10, CTLA4, TGFB1, IDO1, ENTPD1, NT5E, IL22, AREG, IL35, GARP, CD274, FOXP3, IKZF2, EOS, IRF4, LEF1, and BACH2, and a modification, e.g., knockdown, of an endogenous TCR gene, e.g., a TRAC gene sequence.

In some embodiments, the engineered T cells or population of T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding IL10 or CTLA4, and a modification, e.g., knockdown, of a TCR gene, e.g., a TRAC gene sequence.

In some embodiments, the engineered T cells or population of T cells comprise a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule, and a modification, e.g., knockdown, of an endogenous TCR gene, e.g., a TRAC gene sequence.

In any of these embodiments, the engineered T cells or population of T cells may further comprise insertion of sequence(s) encoding a targeting receptor as described herein, e.g., a CAR, e.g., a CAR targeting MAdCAM-1.

In some embodiments, the engineered T cells or population of T cells comprise a modification, e.g., knockdown, of a TRC gene sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous TRC gene sequence. In some embodiments, TRC is decreased by at least 50%, 55%, 60%, 65%, preferably at least 70%, 75%, 80%, 85%, 90%, 95%, or to below the limit of detection of the assay as compared to a suitable control, e.g., wherein the TRC gene has not been modified. Assays for TRC protein and mRNA expression are known in the art.

In some embodiments, the engineered T cells or population of T cells comprise an insertion of sequence(s) encoding a targeting receptor by gene editing, e.g., as assessed by sequencing, e.g., NGS.

In some embodiments, a population of T cells comprises T cells that are engineered to comprise a modification, e.g., knockdown, of an endogenous nucleic acid sequence encoding an IFNG, a modification, e.g., knockdown, of an endogenous nucleic acid sequence encoding a TNFA, insertion of sequences encoding a regulatory T cell promoting molecule, and a modification, e.g., knockdown, of at least one TCR gene sequence. In some embodiments, at least 50%, 55%, 60%, 65%, preferably at least 70%, 75%, 80%, 85%, 90%, 95%, or 100% of the T cells in the population of T cells are engineered to comprise a modification, e.g., knockdown, of at least one TCR gene sequence, e.g., as assessed by sequencing, e.g., NGS.

In some embodiments, guide RNAs that specifically target sites within the TCR genes, e.g., TRAC gene, are used to provide a modification, e.g., knockdown, of the TCR genes.

In some embodiments, the TCR gene is modified, e.g., knocked down, in a T cell using a guide RNA with an RNA-guided DNA binding agent. In some embodiments, disclosed herein are T cells engineered by inducing a break (e.g., double-stranded break (DSB) or single-stranded break (nick)) within the TCR genes of a T cell, e.g., using a guide RNA with an RNA-guided DNA-binding agent (e.g., a CRISPR/Cas system). The methods may be used in vitro or ex vivo, e.g., in the manufacture of cell products for suppressing immune response.

In some embodiments, the guide RNAs mediate a target-specific cutting by an RNA-guided DNA-binding agent (e.g., Cas nuclease) at a site described herein within a TCR gene. It will be appreciated that, in some embodiments, the guide RNAs comprise guide sequences that bind to, or are capable of binding to, said regions.

### D. Guide RNA

In any of the embodiments herein, the guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the sgRNA comprises one or more linkages between nucleotides that is not a phosphodiester linkage.

In each of the composition, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown herein, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA.” The sgRNA may comprise a crRNA (or a portion thereof) comprising a guide sequence shown herein covalently linked to a trRNA. The sgRNA may comprise 15, 16, 17, 18, 19, or 20 contiguous nucleotides of a guide sequence shown herein. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild-type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, the target sequence or region may be complementary to the guide sequence of the guide RNA. In some embodiments, the degree of complementarity or identity between a guide sequence of a guide RNA and its corresponding target sequence may be 75%, 80%, 85%, 90%, 95%, or 100%. In some embodiments, the target sequence and the guide sequence of the gRNA may be 100% complementary or identical. In other embodiments, the target sequence and the guide sequence of the gRNA may contain one mismatch. For example, the target sequence and the guide sequence of the gRNA may contain 1, 2, 3, 4, or 5 mismatches, where the total length of the guide sequence is about 20, or 20. In some embodiments, the target sequence and the guide sequence of the gRNA may contain 1-4 mismatches where the guide sequence is about 20, or 20 nucleotides.

In any of the embodiments herein, each of the guide sequences herein may further comprise additional nucleotides to form a crRNA or guide RNA, e.g., with the following exemplary nucleotide sequence following the guide sequence at its 3′ end: GUUUUAGAGCUAUGCUGUUUUG (SEQ ID NO: 134) in 5′ to 3′ orientation. In the case of a sgRNA, the above guide sequences may further comprise additional nucleotides to form a sgRNA, e.g., with the following exemplary nucleotide sequence following the 3′ end of the guide sequence:

(SEQ ID NO: 135)
GUUUUAGAGCUAGAAAUAGCAAGUUAAAAUAAGGCUAGUCCGUUAUCAA
CUUGAAAAAGUGGCACCGAGUCGGUGCUUUU
or
(SEQ ID NO: 136)
GUUUUAGAGCUAGAAAUAGCAAGUUAAAAUAAGGCUAGUCCGUUAUCAA
CUUGAAAAAGUGGCACCGAGUCGGUGC

In any embodiments, the guide RNAs disclosed herein bind to a region upstream of a propospacer adjacent motif (PAM). As would be understood by those of skill in the art, the PAM sequence occurs on the strand opposite to the strand that contains the target sequence and varies with the CRISPR/Cas system. That is, the PAM sequence is on the complement strand of the target strand (the strand that contains the target sequence to which the guide RNA binds). In some embodiments, the PAM is selected from the group consisting of NGG, NNGRRT, NNGRR(N), NNAGAAW, NNNNG(A/C)TT, and NNNNRYAC.

In some embodiments, the guide RNA sequences provided herein are complementary to a sequence adjacent to a PAM sequence.

In some embodiments, the guide RNA sequence comprises a sequence that is complementary to a sequence within a genomic region selected from the tables herein according to coordinates in human reference genome hg38. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides from within a genomic region selected from the tables herein. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides spanning a genomic region selected from the tables herein.

The guide RNAs disclosed herein mediate a target-specific cutting resulting in a double-stranded break (DSB). The guide RNAs disclosed herein mediate a target-specific cutting resulting in a single-stranded break (SSB or nick).

### E. Chemically Modified gRNA

In any of the embodiments herein, the gRNA may be chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA. Certain gRNAs comprise at least one modified residue at or near the 5′ end and 3′ end of the RNA.

In some embodiments, the guide RNAs disclosed herein comprise one of the modification patterns disclosed in WO2018/107028 the contents of which are hereby incorporated by reference in relevant part. In some embodiments, the guide RNAs disclosed herein comprise one of the structures/modification patterns disclosed in US20170114334, which are hereby incorporated by reference. In some embodiments, the guide RNAs disclosed herein comprise one of the structures/modification patterns disclosed in WO2017/136794, WO2017004279, US2018187186, US2019048338, which are hereby incorporated by reference.

### F. mRNAs Encoding RNA-Guided DNA-Binding Agents

In some embodiments, a cell or method comprises an mRNA comprising an open reading frame (ORF) encoding an RNA-guided DNA-binding agent, such as a Cas nuclease as described herein. Cas9 ORFs are provided herein and are known in the art. As one example, the Cas9 ORF can be codon optimized, such that coding sequence includes one or more alternative codons for one or more amino acids. An “alternative codon” as used herein refers to variations in codon usage for a given amino acid, and may or may not be a preferred or optimized codon (codon optimized) for a given expression system. Preferred codon usage, or codons that are well-tolerated in a given system of expression, is known in the art. The Cas9 coding sequences, Cas9 mRNAs, and Cas9 protein sequences of WO2013/176772, WO2014/065596, WO2016/106121, and WO2019/067910 are hereby incorporated by reference. In particular, the ORFs and Cas9 amino acid sequences of the table at paragraph [0449] WO2019/067910, and the Cas9 mRNAs and ORFs of paragraphs [0214]-[0234] of WO2019/067910 are hereby incorporated by reference.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, for example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Capi, the riboses of the first and second transcribed nucleotides of the mRNA comprise a 2′-methoxy and a 2′-hydroxyl, respectively. See, e.g., CleanCap′ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133). In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115.

In some embodiments, the mRNA further comprises a poly-adenylated (poly-A) tail. In some embodiments, the poly-A tail comprises 20, 30, 40, 50, 60, 70, 80, 90, or 100 adenines (SEQ ID NO: 147), optionally up to 300 adenines (SEQ ID NO: 148). In some embodiments, the poly-A tail comprises 95, 96, 97, 98, 99, or 100 adenine nucleotides (SEQ ID NO: 149).

### G. T Cells for Engineering

The engineered cells provided herein are prepared from a population of cells enriched for CD4+ T cells. Such cells can be readily obtained from fresh leukopak samples, commercially available from various sources including, e.g., StemCell Technologies. CD4+ T cells can be isolated using commercially available kits using routine methods, e.g., by negative selection using the human CD4+ T cell isolation kit. However, methods of preparation of CD4+ T cells from other sources are also known in the art. For example, multipotent cells such as hematopoietic stem cell (HSCs such as those isolated from bone marrow or cord blood), hematopoietic progenitor cells (e.g., lymphoid progenitor cell), or mesenchymal stem cells (MSC) can be used to obtain CD4+ T cells. Multipotent cells are capable of developing into more than one cell type, but are more limited than pluripotent cells in breadth of differentiation. The multipotent cells may be derived from established cell lines or isolated from human bone marrow or umbilical cords. For example, the HSCs may be isolated from a patient or a healthy donor following G-CSF-induced mobilization, plerixafor-induced mobilization, or a combination thereof. To isolate HSCs from the blood or bone marrow, the cells in the blood or bone marrow may be panned by antibodies that bind unwanted cells, such as antibodies to CD4 and CD8 (T cells), CD45 (B cells), GR-I (granulocytes), and lad (differentiated antigen-presenting cells) (see, e.g., Inaba, et al. (1992) J Exp Med. 176: 1693-1702). Methods to promote differentiation into CD4+ T cells are known in the art.

### III. Method of Delivery

The guide RNA, RNA-guided DNA binding agents (e.g., Cas nuclease), and nucleic acid sequences disclosed herein can be delivered to a cell or population of cells, in vitro or ex vivo, for the production of engineered T cells comprising a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule, e.g., IL10, CTLA4; and optionally further comprising insertion of sequence(s) encoding a targeting receptor, e.g., a CAR, and optionally further comprising a modification, e.g., knockdown, of TCR sequence(s), using various known and suitable methods available in the art. The guide RNA, RNA-guided DNA binding agents, and nucleic acid constructs can be delivered individually or together in any combination, using the same or different delivery methods as appropriate.

Conventional viral and non-viral based gene delivery methods can be used to introduce the guide RNA as well as the RNA-guided DNA-binding agent and donor construct in cells (e.g., mammalian cells) and target tissues. As further provided herein, non-viral vector delivery systems nucleic acids such as non-viral vectors, plasmid vectors, and, e.g., naked nucleic acid, and nucleic acid complexed with a delivery vehicle such as a liposome, lipid nanoparticle (LNP), or poloxamer. Viral vector delivery systems include DNA and RNA viruses.

Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

Various delivery systems (e.g., vectors, liposomes, LNPs) containing the guide RNAs, RNA-guided DNA binding agent, and donor construct, singly or in combination, can also be administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and well known to those of skill in the art.

In certain embodiments, the present disclosure provides DNA or RNA vectors encoding any of the compositions disclosed herein e.g., guide RNAs comprising any one or more of the guide sequences described herein, e.g., for modifying (e.g., knocking down) IFNG and TNFA or a donor construct comprising sequence(s) encoding a regulatory T cell promoting molecule, e.g., IL10, or a targeting receptor, e.g., a CAR, e.g., a MAdCAM-1 CAR. In some embodiments, the vector also comprises a sequence encoding an RNA-guided DNA binding agent. In certain embodiments, the invention comprises DNA or RNA vectors encoding any one or more of the compositions described herein, or in any combination. In some embodiments, the vectors further comprise, e.g., promoters, enhancers, and regulatory sequences. In some embodiments, the vector that comprises a guide RNA comprising any one or more of the guide sequences described herein also comprises one or more nucleotide sequence(s) encoding a crRNA, a trRNA, or a crRNA and trRNA, as disclosed herein.

In certain embodiments, the present disclosure provides DNA or RNA vectors encoding a regulatory T cell promoting molecules and a targeting receptor. Such vectors allow for selection of cells based on the presence of the receptor for cells that also contain a coding sequence for the regulatory T cell promoting molecule. Positive and negative selection methods based on the presence of cell surface molecules are known in the art.

In some embodiments, the vector comprises a nucleotide sequence encoding a guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA may be located on the same vector comprising the nucleotide sequence encoding an RNA-guided DNA-binding agent such as a Cas protein. In some embodiments, expression of the guide RNA and of the RNA-guided DNA-binding agent such as a Cas protein may be driven by their own corresponding promoters. In some embodiments, expression of the guide RNA may be driven by the same promoter that drives expression of the RNA-guided DNA-binding agent such as a Cas protein. In some embodiments, the guide RNA and the RNA-guided DNA-binding agent such as a Cas protein transcript may be contained within a single transcript. For example, the guide RNA may be within an untranslated region (UTR) of the RNA-guided DNA-binding agent such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the RNA-guided DNA-binding agent such as a Cas protein and the guide RNA from the same vector in close temporal proximity may facilitate more efficient formation of the CRISPR RNP complex.

In some embodiments, the nucleotide sequence encoding the guide RNA or RNA-guided DNA-binding agent may be located on the same vector comprising the construct that comprises the sequence encoding the regulatory T cell promoting molecule, e.g., IL10, CTLA4; or targeting receptor, e.g., a CAR, e.g., a MAdCAM-1 CAR. In some embodiments, proximity of the construct comprising the sequence encoding the regulatory T cell promoting molecule, e.g., IL10, CTLA4; or targeting receptor, e.g., a CAR, e.g., a MAdCAM-1 CAR and the guide RNA (or the RNA-guided DNA binding agent) on the same vector may facilitate more efficient insertion of the construct into a site of insertion created by the guide RNA/RNA-guided DNA binding agent.

In certain embodiments, DNA and RNA vectors can include more than one open reading frame for expression under a single promoter, either present in the vector or at the genomic insertion site. In such embodiments, a coding sequence for a self-cleaving peptide can be included between the open reading frames. The self-cleaving peptide may be, for example, a 2A peptide, for example, a P2A peptide, an E2A peptide, a F2A peptide, or a T2A peptide.

In some embodiments, the vector comprises one or more nucleotide sequence(s) encoding a sgRNA and an mRNA encoding an RNA-guided DNA binding agent, which can be a Cas protein, such as Cas9 or Cpf1. In some embodiments, the vector comprises one or more nucleotide sequence(s) encoding a crRNA, a trRNA, and an mRNA encoding an RNA-guided DNA binding agent, which can be a Cas protein, such as, Cas9 or Cpf1. In one embodiment, the Cas9 is from Streptococcuspyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector comprises a donor construct comprising a sequence that encodes the regulatory T cell promoting molecule, e.g., IL10, or targeting receptor, e.g., a CAR, e.g., MAdCAM-1, as disclosed herein. In some embodiments, in addition to the donor construct disclosed herein, the vector may further comprise nucleic acids that encode the guide RNAs described herein or nucleic acid encoding an RNA-guided DNA-binding agent (e.g., a Cas nuclease such as Cas9). In some embodiments, a nucleic acid encoding an RNA-guided DNA-binding agent are each or both on a separate vector from a vector that comprises the donor construct disclosed herein. In any of the embodiments, the vector may include other sequences that include, but are not limited to, promoters, enhancers, regulatory sequences, as described herein. In some embodiments, the promoter does not drive the expression of the regulatory T cell promoting molecule, e.g., IL10, or targeting receptor, e.g., a CAR, e.g., MAdCAM-1, of the donor construct. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease (e.g., Cas9). In some embodiments, the vector comprises one or more nucleotide sequence(s) encoding a crRNA, a trRNA, and an mRNA encoding an RNA-guided DNA nuclease, which can be a Cas nuclease, such as, Cas9. In some embodiments, the Cas9 is from Streptococcuspyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild-type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a may be reduced. In some embodiments, viral genes (such as, e.g., integrase) that promote integration of the viral sequence into a genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a cell or population of cells along with the vector system described herein. In other embodiments, the virus may be helper-free. For example, the virus may be capable of amplifying and packaging the vectors without a helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector.

In some embodiments, “AAV” refers all serotypes, subtypes, and naturally-occurring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV 11, AAV12, rh10, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV 11, AAV12, rh10, and hybrids thereof, avian AAV, bovine AAV, canine AAV, equine AAV, primate AAV, nonprimate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV).

In some embodiments, the lentivirus may be integrating. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or other vectors, which have smaller cloning capacity, it may be necessary to use more than one vector to deliver all the components of a vector system as disclosed herein. For example, one AAV vector may contain sequences encoding an RNA-guided DNA-binding agent such as a Cas protein (e.g., Cas9), while a second AAV vector may contain one or more guide sequences.

In some embodiments, the vector system may be capable of driving expression of one or more nuclease components in a cell. In some embodiments, the vector does not comprise a promoter that drives expression of one or more coding sequences once it is integrated in a cell (e.g., uses the cell's endogenous promoter such as when inserted at specific genomic loci of the cell, as exemplified herein). Suitable promoters to drive expression in different types of cells, e.g., CD4+ T cells, are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding an RNA-guided DNA-binding agent such as a Cas protein (e.g., Cas9) described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the vector may comprise any one or more of the constructs comprising a sequence encoding the regulatory T cell promoting molecule, e.g., IL10, CTLA4; or targeting receptor, e.g., a CAR, e.g., a MAdCAM-1 CAR, as described herein. In some embodiments, the sequence of the regulatory T cell promoting molecule, e.g., IL10, CTLA4; or targeting receptor, e.g., a CAR, e.g., a MAdCAM-1 CAR, may be operably linked to at least one transcriptional or translational control sequence. In some embodiments, the sequence of the regulatory T cell promoting molecule, e.g., IL10, CTLA4; or targeting receptor, e.g., a CAR, e.g., a MAdCAM-1 CAR may be operably linked to at least one promoter. In some embodiments, the sequence of the regulatory T cell promoting molecule, e.g., IL10, CTLA4; or targeting receptor, e.g., a CAR, e.g., a MAdCAM-1 CAR, is not linked to a promoter that drives the expression of the heterologous gene.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF 1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On*promoter (Clontech).

In some embodiments, the promoter may be a tissue-specific promoter, e.g., a promoter specific for expression in a T cell.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On*promoter (Clontech).

In additional embodiments, the vector system may comprise tissue-specific promoters.

Non-limiting exemplary viral vector sequences are provided below:

CTLA4 lentiviral insert (nucleotide sequence)
(SEQ ID NO: 137)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAAGCGACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAACTGA
CTLA4 lentiviral insert (amino acid sequence)
(SEQ ID NO: 130)
MACLGFQRHKAQLNLATRTWPCTLLFFLLFIPVFCKAMHVAQPAVVLASSRGIASFV
CEYASPGKATEVRVTVLRQADSQVTEVCAATYMMGNELTFLDDSICTGTSSGNQVN
LTIQGLRAMDTGLYICKVELMYPPPYYLGIGNGTQIYVIDPEPCPDSDELLWILAAVSS
GLFFYSFLLTAVSLSKMLKKRSPLTTGVYVKMPPTEPECEKQFQPYFIPIN*
IL10 lentiviral insert (nucleotide sequence)
(SEQ ID NO: 138)
ATGCACAGCTCAGCACTGCTCTGTTGCCTGGTCCTCCTGACTGGGGTGAGGGCCA
GCCCAGGCCAGGGCACCCAGTCTGAGAACAGCTGCACCCACTTCCCAGGCAACC
TGCCTAACATGCTTCGAGATCTCCGAGATGCCTTCAGCAGAGTGAAGACTTTCTT
TCAAATGAAGGATCAGCTGGACAACTTGTTGTTAAAGGAGTCCTTGCTGGAGGA
CTTTAAGGGTTACCTGGGTTGCCAAGCCTTGTCTGAGATGATCCAGTTTTACCTG
GAGGAGGTGATGCCCCAAGCTGAGAACCAAGACCCAGACATCAAGGCGCATGTG
AACTCCCTGGGGGAGAACCTGAAGACCCTCAGGCTGAGGCTACGGCGCTGTCAT
CGATTTCTTCCCTGTGAAAACAAGAGCAAGGCCGTGGAGCAGGTGAAGAATGCC
TTTAATAAGCTCCAAGAGAAAGGCATCTACAAAGCCATGAGTGAGTTTGACATCT
TCATCAACTACATAGAAGCCTACATGACAATGAAGATACGAAACTGA
IL10 lentiviral insert (amino acid sequence)
(SEQ ID NO: 124)
MHSSALLCCLVLLTGVRASPGQGTQSENSCTHFPGNLPNMLRDLRDAFSRVKTFFQ
MKDQLDNLLLKESLLEDFKGYLGCQALSEMIQFYLEEVMPQAENQDPDIKAHVNSL
GENLKTLRLRLRRCHRFLPCENKSKAVEQVKNAFNKLQEKGIYKAMSEFDIFINYIEA
YMTMKIRN*
FOXP3 lentiviral insert (nucleotide sequence)
(SEQ ID NO: 139)
ATGCCCAACCCCAGGCCTGGCAAGCCCTCGGCCCCTTCCTTGGCCCTTGGCCCAT
CCCCAGGAGCCTCGCCCAGCTGGAGGGCTGCACCCAAAGCCTCAGACCTGCTGG
GGGCCCGGGGCCCAGGGGGAACCTTCCAGGGCCGAGATCTTCGAGGCGGGGCCC
ATGCCTCCTCTTCTTCCTTGAACCCCATGCCACCATCGCAGCTGCAGCTGCCCACA
CTGCCCCTAGTCATGGTGGCACCCTCCGGGGCACGGCTGGGCCCCTTGCCCCACT
TACAGGCACTCCTCCAGGACAGGCCACATTTCATGCACCAGCTCTCAACGGTGGA
TGCCCACGCCCGGACCCCTGTGCTGCAGGTGCACCCCCTGGAGAGCCCAGCCATG
ATCAGCCTCACACCACCCACCACCGCCACTGGGGTCTTCTCCCTCAAGGCCCGGC
CTGGCCTCCCACCTGGGATCAACGTGGCCAGCCTGGAATGGGTGTCCAGGGAGC
CGGCACTGCTCTGCACCTTCCCAAATCCCAGTGCACCCAGGAAGGACAGCACCCT
TTCGGCTGTGCCCCAGAGCTCCTACCCACTGCTGGCAAATGGTGTCTGCAAGTGG
CCCGGATGTGAGAAGGTCTTCGAAGAGCCAGAGGACTTCCTCAAGCACTGCCAG
GCGGACCATCTTCTGGATGAGAAGGGCAGGGCACAATGTCTCCTCCAGAGAGAG
ATGGTACAGTCTCTGGAGCAGCAGCTGGTGCTGGAGAAGGAGAAGCTGAGTGCC
ATGCAGGCCCACCTGGCTGGGAAAATGGCACTGACCAAGGCTTCATCTGTGGCA
TCATCCGACAAGGGCTCCTGCTGCATCGTAGCTGCTGGCAGCCAAGGCCCTGTCG
TCCCAGCCTGGTCTGGCCCCCGGGAGGCCCCTGACAGCCTGTTTGCTGTCCGGAG
GCACCTGTGGGGTAGCCATGGAAACAGCACATTCCCAGAGTTCCTCCACAACAT
GGACTACTTCAAGTTCCACAACATGCGACCCCCTTTCACCTACGCCACGCTCATC
CGCTGGGCCATCCTGGAGGCTCCAGAGAAGCAGCGGACACTCAATGAGATCTAC
CACTGGTTCACACGCATGTTTGCCTTCTTCAGAAACCATCCTGCCACCTGGAAGA
ACGCCATCCGCCACAACCTGAGTCTGCACAAGTGCTTTGTGCGGGTGGAGAGCG
AGAAGGGGGCTGTGTGGACCGTGGATGAGCTGGAGTTCCGCAAGAAACGGAGCC
AGAGGCCCAGCAGGTGTTCCAACCCTACACCTGGCCCCTGATAA
FOXP3 lentiviral insert (amino acid sequence)
(SEQ ID NO: 140)
MPNPRPGKPSAPSLALGPSPGASPSWRAAPKASDLLGARGPGGTFQGRDLRGGAHAS
SSSLNPMPPSQLQLPTLPLVMVAPSGARLGPLPHLQALLQDRPHFMHQLSTVDAHAR
TPVLQVHPLESPAMISLTPPTTATGVFSLKARPGLPPGINVASLEWVSREPALLCTFPN
PSAPRKDSTLSAVPQSSYPLLANGVCKWPGCEKVFEEPEDFLKHCQADHLLDEKGRA
QCLLQREMVQSLEQQLVLEKEKLSAMQAHLAGKMALTKASSVASSDKGSCCIVAA
GSQGPVVPAWSGPREAPDSLFAVRRHLWGSHGNSTFPEFLHNMDYFKFHNMRPPFT
YATLIRWAILEAPEKQRTLNEIYHWFTRMFAFFRNHPATWKNAIRHNLSLHKCFVRV
ESEKGAVWTVDELEFRKKRSQRPSRCSNPTPGP**
Empty lentivector
(SEQ ID NO: 141)
ACGCGTGTAGTCTTATGCAATACTCTTGTAGTCTTGCAACATGGTAACGATGAGT
TAGCAACATGCCTTACAAGGAGAGAAAAAGCACCGTGCATGCCGATTGGTGGAA
GTAAGGTGGTACGATCGTGCCTTATTAGGAAGGCAACAGACGGGTCTGACATGG
ATTGGACGAACCACTGAATTGCCGCATTGCAGAGATATTGTATTTAAGTGCCTAG
CTCGATACATAAACGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGAC
CCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCG
CGCACGGCAAGAGGCGAGGGGCGGCGACTGGTGAGTACGCCAAAAATTTTGACT
AGCGGAGGCTAGAAGGAGAGAGATGGGTGCGAGAGCGTCAGTATTAAGCGGGG
GAGAATTAGATCGCGATGGGAAAAAATTCGGTTAAGGCCAGGGGGAAAGAAAA
AATATAAATTAAAACATATAGTATGGGCAAGCAGGGAGCTAGAACGATTCGCAG
TTAATCCTGGCCTGTTAGAAACATCAGAAGGCTGTAGACAAATACTGGGACAGC
TACAACCATCCCTTCAGACAGGATCAGAAGAACTTAGATCATTATATAATACAGT
AGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAG
CGGCCACTGATCTTCAGACCTGGAGGAGGAGATATGAGGGACAATTGGAGAAGT
GAATTATATAAATATAAAGTAGTAAAAATTGAACCATTAGGAGTAGCACCCACC
AAGGCAAAGAGAAGAGTGGTGCAGAGAGAAAAAAGAGCAGTGGGAATAGGAGC
TTTGTTCCTTGGGTTCTTGGGAGCAGCAGGAAGCACTATGGGCGCAGCGTCAATG
ACGCTGACGGTACAGGCCAGACAATTATTGTCTGGTATAGTGCAGCAGCAGAAC
AATTTGCTGAGGGCTATTGAGGCGCAACAGCATCTGTTGCAACTCACAGTCTGGG
GCATCAAGCAGCTCCAGGCAAGAATCCTGGCTGTGGAAAGATACCTAAAGGATC
AACAGCTCCTGGGGATTTGGGGTTGCTCTGGAAAACTCATTTGCACCACTGCTGT
GCCTTGGAATGCTAGTTGGAGTAATAAATCTCTGGAACAGATTTGGAATCACACG
ACCTGGATGGAGTGGGACAGAGAAATTAACAATTACACAAGCTTAATACACTCC
TTAATTGAAGAATCGCAAAACCAGCAAGAAAAGAATGAACAAGAATTATTGGAA
TTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTT
TGCTGTACTTTCTATAGTGAATAGAGTTAGGCAGGGATATTCACCATTATCGTTTC
AGACCCACCTCCCAACCCCGAGGGGACCCGACAGGCCCGAAGGAATAGAAGAA
GAAGGTGGAGAGAGAGACAGAGACAGATCCATTCGATTAGTGAACGGATCTCGA
CGGTATCGATGGCCGCCCCCTTCACCGAGGGCCTATTTCCCATGATTCCTTCATAT
TTGCATATACGATACAAGGCTGTTAGAGAGATAATTGGAATTAATTTGACTGTAA
ACACAAAGATATTAGTACAAAATACGTGACGTAGAAAGTAATAATTTCTTGGGT
AGTTTGCAGTTTTAAAATTATGTTTTAAAATGGACTATCATATGCTTACCGTAACT
TGAAAGTATTTCGATTTCTTGGCTTTATATATCTTGTGGAAAGGACGAAACACCG
GAGTCTTCTTTTTTGAAGACACTTCGGACTGTAGAACTCTGAACCTCGAGCAATT
TAAAAGAAAAGGGGGGATTGGGGGGTACAGTGCAGGGGAAAGAATAGTAGACA
TAATAGCAACAGACATACAAACTAAAGAATTACAAAAACAAATTACAAAAATTC
AAAATTTCTGCGTTGTTGTCGGTGCTCGTTCTCTGCTCTTCACGCTACTGAATTCA
TCACCGGTTCTTCGAAGGCCTCCGCGCCGGGTTTTGGCGCCTCCCGCGGGCGCCC
CCCTCCTCACGGCGAGCGCTGCCACGTCAGACGAAGGGCGCAGCGAGCGTCCTG
ATCCTTCCGCCCGGACGCTCAGGACAGCGGCCCGCTGCTCATAAGACTCGGCCTT
AGAACCCCAGTATCAGCAGAAGGACATTTTAGGACGGGACTTGGGTGACTCTAG
GGCACTGGTTTTCTTTCCAGAGAGCGGAACAGGCGAGGAAAAGTAGTCCCTTCTC
GGCGATTCTGCGGAGGGATCTCCGTGGGGCGGTGAACGCCGATGATTATATAAG
GACGCGCCGGGTGTGGCACAGCTAGTTCCGTCGCAGCCGGGATTTGGGTCGCGG
TTCTTGTTTGTGGATCGCTGTGATCGTCACTTGGTCTAGACGCCACCATGAGCGG
GGGCGAGGAGCTGTTCGCCGGCATCGTGCCCGTGCTGATCGAGCTGGACGGCGA
CGTGCACGGCCACAAGTTCAGCGTGCGCGGCGAGGGCGAGGGCGACGCCGACTA
CGGCAAGCTGGAGATCAAGTTCATCTGCACCACCGGCAAGCTGCCCGTGCCCTG
GCCCACCCTGGTGACCACCCTCTGCTACGGCATCCAGTGCTTCGCCCGCTACCCC
GAGCACATGAAGATGAACGACTTCTTCAAGAGCGCCATGCCCGAGGGCTACATC
CAGGAGCGCACCATCCAGTTCCAGGACGACGGCAAGTACAAGACCCGCGGCGAG
GTGAAGTTCGAGGGCGACACCCTGGTGAACCGCATCGAGCTGAAGGGCAAGGAC
TTCAAGGAGGACGGCAACATCCTGGGCCACAAGCTGGAGTACAGCTTCAACAGC
CACAACGTGTACATCCGCCCCGACAAGGCCAACAACGGCCTGGAGGCTAACTTC
AAGACCCGCCACAACATCGAGGGCGGCGGCGTGCAGCTGGCCGACCACTACCAG
ACCAACGTGCCCCTGGGCGACGGCCCCGTGCTGATCCCCATCAACCACTACCTGA
GCACTCAGACCAAGATCAGCAAGGACCGCAACGAGGCCCGCGACCACATGGTGC
TCCTGGAGTCCTTCAGCGCCTGCTGCCACACCCACGGCATGGACGAGCTGTACAG
GGGATCCGAGGGCAGAGGAAGCCTTCTAACATGCGGTGACGTGGAGGAGAATCC
CGGCCCTTCCGGGATGACCGAGTACAAGCCCACGGTGCGCCTCGCCACCCGCGA
CGACGTCCCCAGGGCCGTACGCACCCTCGCCGCCGCGTTCGCCGACTACCCCGCC
ACGCGCCACACCGTCGATCCGGACCGCCACATCGAGCGGGTCACCGAGCTGCAA
GAACTCTTCCTCACGCGCGTCGGGCTCGACATCGGCAAGGTGTGGGTCGCGGAC
GACGGCGCCGCGGTGGCGGTCTGGACCACGCCGGAGAGCGTCGAAGCGGGGGC
GGTGTTCGCCGAGATCGGCCCGCGCATGGCCGAGTTGAGCGGTTCCCGGCTGGCC
GCGCAGCAACAGATGGAAGGCCTCCTGGCGCCGCACCGGCCCAAGGAGCCCGCG
TGGTTCCTGGCCACCGTCGGCGTCTCGCCCGACCACCAGGGCAAGGGTCTGGGCA
GCGCCGTCGTGCTCCCCGGAGTGGAGGCGGCCGAGCGCGCCGGGGTGCCCGCCT
TCCTGGAGACCTCCGCGCCCCGCAACCTCCCCTTCTACGAGCGGCTCGGCTTCAC
CGTCACCGCCGACGTCGAGGTGCCCGAAGGACCGCGCACCTGGTGCATGACCCG
CAAGCCCGGTGCCTGAATCTAGGTCGACAATCAACCTCTGGATTACAAAATTTGT
GAAAGATTGACTGGTATTCTTAACTATGTTGCTCCTTTTACGCTATGTGGATACGC
TGCTTTAATGCCTTTGTATCATGCTATTGCTTCCCGTATGGCTTTCATTTTCTCCTC
CTTGTATAAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTGGGGCAT
TGCCACCACCTGTCAGCTCCTTTCCGGGACTTTCGCTTTCCCCCTCCCTATTGCCA
CGGCGGAACTCATCGCCGCCTGCCTTGCCCGCTGCTGGACAGGGGCTCGGCTGTT
GGGCACTGACAATTCCGTGGTGTTGTCGGGGAAATCATCGTCCTTTCCTTGGCTG
CTCGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCT
CTTCCGCGTCTCCGCCTTCGCCCTCAGACGAGTCGGATCTCTCTTTGGGCCGCCTC
CCCGCCTGGTACCTTTAAGACCAATGACTTACAAGGCAGCTGTAGATCTTAGCCA
CTTTTTAAAAGAAAAGGGGGGACTGGAAGGGCTAATTCACTCCCAACGAAGATA
AGATCTGCTTTTTGCTTGTACTGGGTCTCTCTGGTTAGACCAGATCTGAGCCTGGG
AGCTCTCTGGCTAACTAGGGAACCCACTGCTTAAGCCTCAATAAAGCTTGCCTTG
AGTGCTTCAAGTAGTGTGTGCCCGTCTGTTGTGTGACTCTGGTAACTAGAGATCC
CTCAGACCCTTTTAGTCAGTGTGGAAAATCTCTAGCAGTAGTAGTTCATGTCATC
TTATTATTCAGTATTTATAACTTGCAAAGAAATGAATATCAGAGAGTGAGAGGAA
CTTGTTTATTGCAGCTTATAATGGTTACAAATAAAGCAATAGCATCACAAATTTC
ACAAATAAAGCATTTTTTTCACTGCATTCTAGTTGTGGTTTGTCCAAACTCATCAA
TGTATCTTATCATGTCTGGCTCTAGCTATCCCGCCCCTAACTCCGCCCATCCCGCC
CCTAACTCCGCCCAGTTCCGCCCATTCTCCGCCCCATGGCTGACTAATTTTTTTTA
TTTATGCAGAGGCCGAGGCCGCCTCGGCCTCTGAGCTATTCCAGAAGTAGTGAGG
AGGCTTTTTTGGAGGCCTAGACTTTTGCAGAGACCAAATTCGTAATCATGTCATA
GCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTA
ATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTTC
CGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTA
CCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTT
TAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGG
GCTTACCATCTGGCCCCAGTGCTGCAATGATACCGCGAGACCCACGCTCACCGGC
TCCAGATTTATCAGCAATAAACCAGCCAGCCGGAAGGGCCGAGCGCAGAAGTGG
TCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTG
CTCTTGCCCGGCGTCAATACGGGATAATACCGCGCCACATAGCAGAACTTTAAAA
GTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCA
AAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGT
GCCACCTGACGTCTAAGAAACCATTATTATCATGACATTAACCTATAAAAATAGG
CGTATCACGAGGCCCTTTCGTCTCGCGCGTTTCGGTGATGACGGTGAAAACCTCT
GACACATGCAGCTCCCGGAGACGGTCACAGCTTGTCTGTAAGCGGATGCCGGGA
GCAGACAAGCCCGTCAGGGCGCGTCAGCGGGTGTTGGCGGGTGTCGGGGCTGGC
TTAACTATGCGGCATCAGAGCAGATTGTACTGAGAGTGCACCATATGCGGTGTGA
AATACCGCACAGATGCGTAAGGAGAAAATACCGCATCAGGCGCCATTCGCCATT
CAGGCTGCGCAACTGTTGGGAAGGGCGATCGGTGCGGGCCTCTTCGCTATTACGC
CAGCTGGCGAAAGGGGGATGTGCTGCAAGGCGATTAAGTTGGGTAACGCCAGGG
TTTTCCCAGTCACGACGTTGTAAAACGACGGCCAGTGCCAAGCTG
Empty lentivector
(SEQ ID NO: 142)
GCGATCGCAGTAATCAATTACGGGGTCATTAGTTCATAGCCCATATATGGAGTTC
CGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGC
CCGCCTGGCATTATGCCCAGTACATGACCTTATGGGACTTTCCTACTTGGCAGTA
CATCTACGTATTAGTCATCGCTATTACCATGGTGATGCGGTTTTGGCAGTACATC
AATGGGCGTGGATAGCGGTTTGACTCACGGGGATTTCCAAGTCTCCACCCCATTG
ACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGT
CTATATAAGCAGAGCTCGTTTAGTGAACCGGGGTCTCTCTGGTTAGACCAGATCT
GAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAAC
TAGAGATCCCTCAGACCCTTTTAGTCAGTGTGGAAAATCTCTAGCAGTGGCGCCC
GAACAGGGACCTGAAAGCGAAAGGGAAACCAGAGCTCTCTCGACGCAGGACTC
GGCTTGCTGAAGCGCGCACGGCAAGAGGCGAGGGGCGGCGACTGGTGAGTACGC
CAAAAATTTTGACTAGCGGAGGCTAGAAGGAGAGAGATGGGTGCGAGAGCGTCA
GTATTAAGCGGGGGAGAATTAGATCGCGATGGGAAAAAATTCGGTTAAGGCCAG
GGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCA
TTATATAATACAGTAGCAACCCTCTATTGTGTGCATCAAAGGATAGAGATAAAAG
ACACCAAGGAAGCTTTAGACAAGATAGAGGAAGAGCAAAACAAAAGTAAGACC
ACCGCACAGCAAGCGGCCGCTGATCTTCAGACCTGGAGGAGGAGATATGAGGGA
CAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAG
TGGGAATAGGAGCTTTGTTCCTTGGGTTCTTGGGAGCAGCAGGAAGCACTATGGG
CGCAGCCTCAATGACGCTGACGGTACAGGCCAGACAATTATTGTCTGGTATAGTG
CAGCAGCAGAACAATTTGCTGAGGGCTATTGAGGCGCAACAGCATCTGTTGCAA
CTCACAGTCTGGGGCATCAAGCAGCTCCAGGCAAGAATCCTGGCTGTGGAAAGA
TACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGAT
TTGGAATCACACGACCTGGATGGAGTGGGACAGAGAAATTAACAATTACACAAG
CTTAATACACTCCTTAATTGAAGAATCGCAAAACCAGCAAGAAAAGAATGAACA
AGAATTATTGGAATTAGATAAATGGGCAAGTTTGTGGAATTGGTTTAACATAACA
AATTGGCTGTGGTATATAAAATTATTCATAATGATAGTAGGAGGCTTGGTAGGTT
TAAGAATAGTTTTTGCTGTACTTTCTATAGTGAATAGAGTTAGGCAGGGATATTC
ACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAG
TGAACGGATCTCGACGGTATCGGTTAACTTTTAAAAGAAAAGGGGGGATTGGGG
GGTACAGTGCAGGGGAAAGAATAGTAGACATAATAGCAACAGACATACAAACT
AAAGAATTACAAAAACAAATTACAAAAATTCAAAATTTTGGCTCCCGATCGTTGC
GTTACACACACAATTACTGCTGATCGAGTGTAGCCTTCGAATGAAAGACCCCACC
TGTAGGTTTGGCAAGATAGCTGCAGTAACGCCATTTTGCAAGGCATGGAAAAAT
ACCAAACCAAGAATAGAGAAGTTCAGATCAAGGGCGGGTACATGAAAATAGCTA
ACGTTGGGCCAAACAGGATATCTGCGGTGAGCAGTTTCGGCCCCGGCCCGGGGC
CAAGAACAGATGGTCACCGCAGTTTCGGCCCCGGCCCGAGGCCAAGAACAGATG
GTCCCCAGATATGGCCCAACCCTCAGCAGTTTCTTAAGACCCATCAGATGTTTCC
AGGCTCCCCCAAGGACCTGAAATGACCCTGCGCCTTATTTGAATTAACCAATCAG
CCTGCTTCTCGCTTCTGTTCGCGCGCTTCTGCTTCCCGAGCTCTATAAAAGAGCTC
ACAACCCCTCACTCGGCGCGCCAGTCCTCCGATTGACTGAGTCGCCCTGATCATT
GTCGATCCTACCATCCACTCGACACACCCGCCAGGGCCCTGCCAAGCTTCCGAGC
TCTCGATATCAAAGGAGGTACCCAACATGGTCAGCAAGGGCGAGGAACTGTTCA
CCGGGGTGGTGCCCATCCTGGTCGAGCTGGACGGCGACGTAAACGGCCACAAGT
TCAGCGTGTCCGGCGAGGGCGAGGGCGATGCCACCTACGGCAAGCTGACCCTGA
AGTTCATCTGTACCACCGGCAAGCTGCCCGTGCCCTGGCCCACCCTCGTGACCAC
CCTGACCTACGGCGTGCAATGCTTCAGCCGCTACCCCGACCACATGAAGCAGCAC
GACTTCTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGCGACA
CCCTGGTGAACCGCATCGAGCTGAAGGGCATCGACTTCAAGGAGGACGGCAACA
TCCTGGGGCACAAGCTGGAGTACAACTACAACAGCCACAACGTCTATATCATGG
CCGACAAGCAGAAGAACGGCATCAAGGTGAACTTCAAGATCCGCCACAACATCG
AGGACGGCAGCGTGCAACTCGCCGACCACTACCAGCAGAACACCCCCATCGGCG
ACGGCCCCGTGCTGCTGCCCGACAACCACTACCTGAGCACCCAGTCCGCCCTGAG
CAAAGACCCCAACGAGAAGCGCGATCACATGGTCCTGCTGGAGTTCGTGACCGC
CGCCGGGATCACTCTCGGCATGGACGAGCTGTACAAGTAGAAGTIGTCTCCTCCT
GCACTGACTGACTGATACAATCGATTTCTGGATCCGCAGGCCTCTGCTAGAAGTT
GTCTCCTCCTGCACTGACTGACTGATACAATCGATTTCTGGATCCGCAGGCCTCT
GCTAGCTTGACTGACTGAGTCGACAATCAACCTCTGGATTACAAAATTTGTGAAA
GATTGACTGGTATTCTTAACTATGTTGCTCCTTTTACGCTATGTGGATACGCTGCT
TTAATGCCTTTGTATCATGCTATTGCTTCCCGTATGGCTTTCATTTTCTCCTCCTTG
TATAAATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGCATTGC
CACCACCTGTCAGCTCCTTTCCGGGACTTTCGCTTTCCCCCTCCCTATTGCCACGG
CGGAACTCATCGCCGCCTGCCTTGCCCGCTGCTGGACAGGGGCTCGGCTGTTGGG
CACTGACAATTCCGTGGTGTTGTCGGGGAAGCTGACGTCCTTTCCATGGCTGCTC
GCCTGTGTTGCCACCTGGATTCTGCGCGGGACGTCCTTCTGCTACGTCCCTTCGGC
CCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCG
CCTGGAATTCGAGCTCGGTACCTTTAAGACCAATGACTTACAAGGCAGCTGTAGA
TCTTAGCCACTTTTTAAAAGAAAAGGGGGGACTGGAAGGGCTAATTCACTCCCA
ACGAAGACAAGATCTGCTTTTTGCTTGTACTGGGTCTCTCTGGTTAGACCAGATC
TGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAAC
TAGAGATCCCTCAGACCCTTTTAGTCAGTGTGGAAAATCTCTAGCAGTCCTGGCC
AACGTGAGCACCGTGCTGACCTCCAAATATCGTTAAGCTGGAGCCTGGGAGCCG
GCCTGGCCCTCCGCCCCCCCCACCCCCGCAGCCCACCCCTGGTCTTTGAATAAAG
TCTGAGTGAGTGGCCGACAGTGCCCGTGGAGTTCTCGTGACCTGAGGTGCAGGG
CCGGCGCTAGGGACACGTCCGTGCACGTGCCGAGGCCCCCTGTGCAGCTGCAAG
GGACAGGCCTAGCCCTGCAGGCCTAACTCCGCCCATCCCGCCCCTAACTCCGCCC
AGTTCCGCCCATTCTCCGCCTCATGGCTGACTAATTTTTTTTATTTATGCAGAGGC
CGAGGCCGCCTCGGCCTCTGAGCTATTCCAGAAGTAGTGAGGACGCTTTTTTGGA
GGCCGAGGCTTTTGCAAAGATCGAACAAGAGACAGGACCTGCAGGTTAATTAAA
TTTAAATCATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACG
TTAAGGGATTTTGGTCATGAGATTATCAAAAAGGATCTTCACCTAGATCCTTTTA
AATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCG
TTCATCCATAGTTGCATTTAAATGGCCGGCCTGGCGCGCCGTTTAAACCTAGATA
TTGATAGTCTGATCGGTCAACGTATAATCGAGTCCTAGCTTTTGCAAACATCTAT
CAAGAGACAGGATCAGCAGGAGGCTTTCGCATGAGTATTCAACATTTCCGTGTCG
CCCTTATTCCCTTTTTTGCGGCATTTTGCCTTCCTGTTTTTGCTCACCCAGAAACGC
TGGTGAAAGTAAAAGATGCTGAAGATCAGTTGGGTGCGCGAGTGGGTTACATCG
AACTGGATCTCAACAGCGGTAAGATCCTTGAGAGTTTTCGCCCCGAAGAACGCTT
TCCAATGATGAGCACTTTTAAAGTTCTGCTATGTGGCGCGGTATTATCCCGTATTG
ACGCCGGGCAAGAGCAACTCGGTCGCCGCATACACTATTCTCAGAATGACTTGGT
TGAGTATTCACCAGTCACAGAAAAGCATCTTACGGATGGCATGACAGTAAGAGA
ATTATGCAGTGCTGCCATAACCATGAGTGATAACACTGCGGCCAACTTACTTCTG
ACAACGATTGGAGGACCGAAGGAGCTAACCGCTTTTTTGCACAACATGGGGGAT
CATGTAACTCGCCTTGATCGTTGGGAACCGGAGCTGAATGAAGCCATACCAAAC
GACGAGCGTGACACCACGATGCCTGTAGCAATGGCAACAACCTTGCGTAAACTA
TTAACTGGCGAACTACTTACTCTAGCTTCCCGGCAACAGTTGATAGACTGGATGG
AGGCGGATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTGCAGCA
CTGGGGCCAGATGGTAAGCCCTCCCGTATCGTAGTTATCTACACGACGGGGAGTC
AGGCAACTATGGATGAACGAAATAGACAGATCGCTGAGATAGGTGCCTCACTGA
TTAAGCATTGGTAACCGATTCTAGGTGCATTGGCGCAGAAAAAAATGCCTGATGC
GACGCTGCGCGTCTTATACTCCCACATATGCCAGATTCAGCAACGGATACGGCTT
CCCCAACTTGCCCACTTCCATACGTGTCCTCCTTACCAGAAATTTATCCTTAAGAT
CCCGAATCGTTTAAAC

The vector comprising: a guide RNA, RNA-binding DNA binding agent, or donor construct comprising a sequence encoding the regulatory T cell promoting molecule, e.g., IL10, CTLA4; or targeting receptor, e.g., a CAR, individually or in any combination, may be delivered by liposome, a nanoparticle, an exosome, or a microvesicle. The vector may also be delivered by a lipid nanoparticle (LNP). One or more guide RNA, RNA-binding DNA binding agent (e.g., mRNA), or donor construct comprising a sequence encoding a heterologous protein, individually or in any combination, may be delivered by LNP, liposome, a nanoparticle, an exosome, or a microvesicle. One or more guide RNA, RNA-binding DNA binding agent (e.g., mRNA), or donor construct comprising a sequence encoding a heterologous protein, individually or in any combination, may be delivered by LNP. In some embodiments, one or more guide RNA and an RNA-guided DNA-binding agent (e.g., mRNA) are delivered by LNP. A donor construct may be delivered by viral vector.

Lipid nanoparticles (LNPs) are a well-known means for delivery of nucleotide and protein cargo, and may be used for delivery of any of the guide RNAs, RNA-guided DNA binding agent, or donor construct disclosed herein.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical and, in more particular embodiments, can comprise an aqueous core, e.g., comprising a substantial portion of RNA molecules), a dispersed phase in an emulsion, micelles, or an internal phase in a suspension (see, e.g., WO2017173054, the contents of which are hereby incorporated by reference in their entirety). Any LNP known to those of skill in the art to be capable of delivering nucleotides to subjects may be utilized.

In some embodiments, provided herein is a method for delivering any of the guide RNAs described herein or donor construct disclosed herein, alone or in combination, to a cell or a population of cells or a subject, wherein any one or more of the components is associated with an LNP. In some embodiments, the method further comprises an RNA-guided DNA-binding agent (e.g., Cas9 or a sequence encoding Cas9).

In some embodiments, provided herein is a composition comprising any of the guide RNAs described herein or donor construct disclosed herein, alone or in combination, with an LNP. In some embodiments, the composition further comprises an RNA-guided DNA-binding agent (e.g., Cas9 or a sequence encoding Cas9).

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO2017/173054, WO2015/095340, and WO2014/136086, as well as references provided therein. In some embodiments, the LNPs comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, LNPs associated with the construct disclosed herein are for use in preparing a cell-based medicament for suppressing immune response. Methods for preparation of cell-based therapeutics and reagents for use in cell based therapeutics are known in the art.

In some embodiments, any of the guide RNAs described herein, RNA-guided DNA binding agents, or donor construct disclosed herein, alone or in combination, whether naked or as part of a vector, is formulated in or administered via a lipid nanoparticle; see e.g., WO2019/067992 or WO2017/173054 the contents of which are hereby incorporated by reference in their entirety.

In some embodiments, an LNP composition is encompassed comprising: an RNA component and a lipid component, wherein the lipid component comprises an amine lipid such as a biodegradable, ionizable lipid. In some instances, the lipid component comprises biodegradable, ionizable lipid, cholesterol, DSPC, and PEG-DMG. In certain embodiments, the lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG. In certain embodiments, the components are present in a 50:38:9:3 molar ratio, respectively. The lipid nucleic acid assemblies may be formulated with a lipid amine to RNA phosphate (N:P) molar ratio of about 6, and a ratio of gRNA to mRNA of 2:1, 1:1, or 1:2 by weight.

It will be apparent that a guide RNA, an RNA-guided DNA-binding agent (e.g., Cas nuclease or a nucleic acid encoding a Cas nuclease), and a donor construct comprising a sequence encoding the regulatory T cell promoting molecule, e.g., IL10, or targeting receptor, e.g., a CAR can be delivered using the same or different systems. For example, the guide RNA, Cas nuclease, and construct can be carried by the same vector (e.g., AAV). Alternatively, the Cas nuclease (as a protein or mRNA) or gRNA can be carried by a plasmid or LNP, while the donor construct can be carried by a vector such as AAV.

The different delivery systems can be delivered simultaneously or in any sequential order. In some embodiments, the donor construct, guide RNA, and Cas nuclease can be delivered simultaneously, e.g., in one vector, two vectors, individual vectors, one LNP, two LNPs, individual LNPs, or a combination thereof. In some embodiments, the donor construct can be delivered as a vector or associated with a LNP, prior to (e.g., about 1, 2, 3, 4, 5, 6, 7, 8, 9, 10, 11, 12, 13, 14, or more days) delivering the guide RNA or Cas nuclease, as a vector or associated with a LNP singly or together or as a ribonucleoprotein (RNP). As a further example, the guide RNA and Cas nuclease, as a vector or associated with a LNP singly or together or as a ribonucleoprotein (RNP), can be delivered prior to (e.g., about 1, 2, 3, 4, 5, 6, 7, 8, 9, 10, 11, 12, 13, 14, or more days) delivering the construct, as a vector or associated with a LNP.

### IV. Method of Engineering T Cells

The disclosure provides methods of engineering T cells to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence. The disclosure provides methods of engineering T cells to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding IL10 under control of a promoter sequence. The disclosure provides methods of engineering T cells to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding CTLA4 under control of a promoter sequence. The disclosure provides methods of engineering T cells to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding IL10 and CTLA4, each under control of a promoter sequence.

In some embodiments, the methods comprise engineering T cells to comprise a modification, e.g., knockdown, of an endogenous nucleic acid sequence encoding an IFNG, a modification, e.g., knockdown, of an endogenous nucleic acid sequence encoding a TNFA, insertion into the cell of heterologous sequence(s) encoding a regulatory T cell promoting molecule, e.g., IL10 or CTLA4, and further comprise a modification, e.g., knockdown, of TCR sequence(s).

In some embodiments, the methods comprise engineering T cells to comprise a modification, e.g., knockdown, of an endogenous nucleic acid sequence encoding an IFNG, a modification, e.g., knockdown, of an endogenous nucleic acid sequence encoding a TNFA, insertion into the cell of heterologous sequence(s) encoding a regulatory T cell promoting molecule, e.g., IL10 or CTLA4, and further comprise insertion into the cell of heterologous sequence(s) encoding a targeting receptor, e.g., a CAR.

In some embodiments, the methods comprise engineering T cells to comprise a modification, e.g., knockdown, of an endogenous nucleic acid sequence encoding an IFNG, a modification, e.g., knockdown, of an endogenous nucleic acid sequence encoding a TNFA, insertion into the cell of heterologous sequence(s) encoding a regulatory T cell promoting molecule, e.g., IL10 or CTLA4, a modification, e.g., knockdown, of TCR sequence(s), and insertion into the cell of heterologous sequence(s) encoding a targeting receptor, e.g., a CAR.

In some embodiments, the modification, e.g., knockdown, of an endogenous nucleic acid sequence encoding an IFNG, the modification, e.g., knockdown, of an endogenous nucleic acid sequence encoding a TNFA, insertion into the cell of heterologous sequence(s) encoding a regulatory T cell promoting molecule, optional knockdown of a TCR gene and optional insertion into the cell of a targeting receptor, e.g., a CAR, are engineered using the CRISPR/Cas system and the guide RNAs disclosed herein.

In these embodiments, the regulatory T cell promoting molecule to be inserted may be provided via a donor construct. The regulatory T cell promoting molecule provided via a donor construct may be selected from IL10, CTLA4, TGFB1, IDO1, ENTPD1, NT5E, IL22, AREG, IL35, GARP, CD274, FOXP3, IKZF2, EOS, IRF4, LEF1, and BACH2, and a modification, e.g., knockdown, of TCR gene sequence(s).

In these embodiments, the targeting receptor to be inserted may be provided via a donor construct. In some embodiments, the targeting receptor may be a chimeric antigen receptor (CAR), a T-cell receptor (TCR), or a receptor for a cell surface molecule operably linked through at least a transmembrane domain in an internal signaling domain capable of activating a T cell upon binding of the extracellular receptor portion. In some embodiments, the targeting receptor may be a receptor present on the surface of a cell, e.g., a T cell, to permit binding of the cell to a target site, e.g., a specific cell or tissue in an organism. In these embodiments, the targeting receptor is a CAR capable of targeting MAdCAM-1.

Suitable gene editing systems for engineering the T cells to comprise insertions and modifications, e.g., knockdowns, are disclosed herein and known in the art. In some embodiments, the gene editing systems include but are not limited to the CRISPR/Cas system; zinc finger nuclease (ZFN) system; transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence, such as a CRISPR/Cas9 system. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, TALEs of genome targeting Nature Biotech. 29:135-136 (2011)). The restriction enzymes can be introduced into cells, for use in gene editing or for genome editing in situ, a technique known as genome editing with engineered nucleases. Such methods and compositions for use therein are known in the art. See, e.g., WO2019147805, WO2014040370, WO2018073393, the contents of which are hereby incorporated in their entireties.

Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

RNA interference (RNAi) is a biological process in which RNA molecules inhibit gene expression or translation, by neutralizing targeted mRNA molecules. Small interfering RNAs (siRNAs) are central to RNA interference. RNAs are the direct products of genes, and these small RNAs (typically each strand being 19-23 nucleotides in length forming a duplex of 19-21 nucleotides) can direct the RNA induced silencing (RISC) complex to degrade messenger RNA (mRNA) molecules and thus decrease their activity by preventing translation, via post-transcriptional gene silencing. Short hairpin RNAs (shRNAs) are siRNAs that are a single RNA strand wherein the strands forming the duplex region have a hairpin structure, often generated by transcription from an expression vector. RNAi can also be accomplished by longer RNA duplex structures referred to as Dicer substrate molecules, which are cleaved by the enzyme Dicer before being loaded into RISC to promote target mRNA cleavage. Such methods and compositions for use are known in the art. In the compositions and methods provided herein, it is preferred that the RNA molecule to promote RNA interference is provided as an expression vector for durability, see, e.g., WO2018208837, the contents of which are hereby incorporated in their entireties. In some embodiments, RNAi is used with an expression vector.

It will be appreciated that the present disclosure contemplates methods of insertion performed with or without the guide RNAs disclosed herein (e.g., using a ZFN system to cause a break in a target DNA sequence, creating a site for insertion of the construct). For methods that use guide RNAs disclosed herein, the methods include the use of the CRISPR/Cas system to modify, e.g., knockdown, a nucleic acid sequence encoding TNFA, IFNG, or TCR. It will also be appreciated that the present disclosure contemplates methods of modifying, e.g., knocking down, TNFA, IFNG, or TCR, which can be performed without the guide RNAs disclosed herein (e.g., using a ZFN system to cause a break in a target DNA sequence, creating a site for insertion of the construct).

In some embodiments, the donor construct comprising the sequence for insertion, e.g., a sequence encoding IL10 or CTLA4, is inserted at a genomic locus for a sequence that is targeted for modification, e.g., knockdown, e.g., a TCR gene.

In some embodiments, a CRISPR/Cas system (e.g., a guide RNA and RNA-guided DNA binding agent) can be used to create a site of insertion at a desired locus within a genome, at which site a donor construct comprising a sequence encoding IL10, CTLA4, or targeting receptor, e.g., a CAR, e.g., a MAdCAM-1 CAR, disclosed herein can be inserted to express IL10, CTLA4, or a CAR, e.g., a MAdCAM-1 CAR. The targeting receptor, e.g., a CAR, e.g., a MAdCAM-1 CAR, IL10, or CTLA4 may be heterologous with respect to its insertion site or locus, for example a safe harbor locus or a TCR locus from which IL10, CTLA4, or targeting receptor, e.g., a CAR, e.g., MAdCAM-1 CAR, is not normally expressed, as described herein. In some embodiments, a guide RNA described herein can be used according to the present methods with an RNA-guided DNA-binding agent (e.g., Cas nuclease) to create a site of insertion, at which site a donor construct comprising a sequence encoding IL10, CTLA4, or targeting receptor, e.g., a CAR, e.g., a MAdCAM-1 CAR can be inserted to express IL10, CTLA4, or a CAR, e.g., a MAdCAM-1 CAR. The guide RNAs for insertion of IL10, CTLA4, or targeting receptor, e.g., a CAR, e.g., a MAdCAM-1 CAR, into specific genomic loci, are exemplified and described herein.

In some embodiments, CD4+ T cells are engineered by transduction (e.g., using viral or non-viral delivery) with a gRNA (e.g., gRNA targeting IFNG, TNFA, or TCR for knockdown), an RNA-guided DNA-binding agent (e.g., Cas nuclease), a donor construct. In some embodiments, the engineered T cells are: 1) transduced with a gRNA targeting a nucleic acid sequence encoding a pro-inflammatory cytokine, e.g., IFNG or TNFA, an RNA guided DNA binding agent (e.g., Cas nuclease), and 2) transduced with a donor construct comprising nucleic acid sequence(s) encoding a regulatory T cell promoting molecule, e.g., IL10 or CTLA4, and a targeting receptor, e.g., a CAR, e.g., a MAdCAM-1 CAR. In certain embodiments, the engineered cells are selected for expression of the targeting receptor.

In some embodiments, CD4+ T cells are engineered by transduction with a gRNA (e.g., gRNA targeting IFNG, TNFA, or TCR for knockdown), an RNA-guided DNA-binding agent (e.g., Cas nuclease) and a donor construct. In some embodiments, the engineered T cells are: 1) transduced with a donor construct comprising nucleic acid sequence(s) encoding a regulatory T cell promoting molecule, e.g., IL10 or CTLA4, and a targeting receptor, e.g., a CAR, e.g., a MAdCAM-1 CAR, and 2) transduced with a gRNA targeting a nucleic acid sequence encoding a pro-inflammatory cytokine, e.g., IFNG or TNFA, an RNA guided DNA binding agent (e.g., Cas nuclease). In certain embodiments, the engineered cells are selected for expression of the targeting receptor.

In some embodiments, CD4+ T cells are engineered by transduction with a gRNA (e.g., gRNA targeting IFNG, TNFA, or TCR for knockdown), an RNA-guided DNA-binding agent (e.g., Cas nuclease), a donor construct. In some embodiments, the engineered T cells are: 1) transduced with a donor construct comprising nucleic acid sequence(s) encoding a regulatory T cell promoting molecule, e.g., IL10 or CTLA4, and a targeting receptor, e.g., a CAR, e.g., a MAdCAM-1 CAR, and 2) transduced with a gRNA targeting a nucleic acid sequence encoding a pro-inflammatory cytokine, e.g., IFNG or TNFA, an RNA guided DNA binding agent (e.g., Cas nuclease).

As described herein, the donor construct comprising a sequence encoding IL10, CTLA4, or a targeting receptor e.g., a CAR, guide RNA (e.g., gRNA targeting IFNG, TNFA, or TCR for knockdown), and RNA-guided DNA-binding agent can be delivered using any suitable delivery system and method known in the art. In some embodiments, the guide RNA and Cas nuclease are associated with an LNP and delivered to the cell or the population of cells prior to delivering the donor construction comprising a sequence encoding IL10, CTLA4, or a targeting receptor, e.g., a CAR. In some embodiments, the guide RNA and Cas nuclease are associated with an LNP and delivered to the cell or the population of cells after delivering the donor construction comprising a sequence encoding IL10, CTLA4, or a targeting receptor, e.g., a CAR.

In some embodiments, administration of the gRNAs, donor construct, and RNA-guided DNA binding agents described herein to a naturally occurring T cell is capable of converting the naturally occurring T cell, e.g., a CD4+ T cell, to a cell that exhibits the characteristics, e.g., immune response suppressive characteristics, of a regulatory T cell.

gRNAs, donor constructs, and RNA-guided DNA binding agents for modifying, e.g., knocking down, IFNG, TNFA, or TCR gene expression or inserting a sequence encoding IL10, CTLA4, or a targeting receptor, e.g., a CAR, e.g., a MAdCAM-1 CAR, may be introduced to a conventional T cell or population of conventional T cells to generate the engineered T cells or population of T cells described herein.

Methods of using various RNA-guided DNA-binding agents, e.g., a nuclease, such as a Cas nuclease, e.g., Cas9, are also well known in the art. While the use of a CRISPR/Cas system is exemplified herein, it will be appreciated that suitable variations to the system can also be used. It will be appreciated that, depending on the context, the RNA-guided DNA-binding agent can be provided as a nucleic acid (e.g., DNA or mRNA), such as the mRNAs encoding an RNA-guided DNA-binding agent provided above, or as a protein. In some embodiments, the present method can be practiced in a cell that already comprises or expresses an RNA-guided DNA-binding agent.

In some embodiments, the RNA-guided DNA-binding agent, such as a Cas9 nuclease, has cleavase activity, which can also be referred to as double-strand endonuclease activity. In some embodiments, the RNA-guided DNA-binding agent, such as a Cas9 nuclease, has nickase activity, which can also be referred to as single-strand endonuclease activity. In some embodiments, the RNA-guided DNA-binding agent comprises a Cas nuclease. Examples of Cas nucleases include those of the type II CRISPR systems of S. pyogenes, S. aureus, and other prokaryotes (see, e.g., the list in the next paragraph), and variant or mutant (e.g., engineered, non-naturally occurring, naturally occurring, or other variant) versions thereof. See, e.g., US2016/0312198 A1; US 2016/0312199 A1.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcuspyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA-binding agent is called a ribonucleoprotein complex (RNP). In some embodiments, the RNA-guided DNA-binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild-type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild-type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)).

In some embodiments, a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA-binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 143) or PKKKRRV (SEQ ID NO: 144). In some embodiments, the NLS may be a bipartite sequence, such as the NLS of nucleoplasmin, KRPAATKKAGQAKKKK (SEQ ID NO: 145). In a specific embodiment, a single PKKKRKV (SEQ ID NO: 143) NLS may be linked at the C-terminus of the RNA-guided DNA-binding agent. One or more linkers are optionally included at the fusion site.

### V. Method of Treatment

The disclosure provides methods for suppressing an immune response in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence. The disclosure provides methods for suppressing an immune response in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding IL10 under control of a promoter sequence. The disclosure provides methods for suppressing an immune response in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding CTLA4 under control of a promoter sequence. The disclosure provides methods for suppressing an immune response in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding IL10 and CTLA4.

The disclosure provides methods for treating an autoimmune disorder in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence. The disclosure provides methods for treating an autoimmune disorder in a subject, comprising administering engineered T cells a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding IL10 under control of a promoter sequence. The disclosure provides methods for treating an autoimmune disorder in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding CTLA4 under control of a promoter sequence. The disclosure provides methods for treating an autoimmune disorder in a subject, comprising administering engineered T cells a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding IL10 and CTLA4, each under control of a promoter sequence.

The disclosure provides methods for treating GvHD in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence. The disclosure provides methods for treating GvHD in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding TL10 under control of a promoter sequence. The disclosure provides methods for treating GvHD in a subject, comprising administering engineered T cells comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding CTLA4 under control of a promoter sequence. The disclosure provides methods for treating GvHD in a subject, comprising administering engineered T cells a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding IL10 and CTLA4, each under control of a promoter sequence.

In some embodiments, the methods comprise administering engineered T cells comprising a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule, and further comprising a modification, e.g., knockdown, of TCR sequence(s).

In some embodiments, the methods comprise administering engineered T cells comprising a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule, and further comprising insertion of sequence(s) encoding a targeting receptor, e.g., a CAR.

In some embodiments, the targeting receptor, e.g., a CAR, is capable of targeting engineered T cells to the gastrointestinal system, e.g., the targeting receptor is a CAR targeting MAdCAM-1, e.g., for suppressing immune responses, including inflammation, in disorders such as inflammatory bowel disease, ulcerative colitis, or Crohn's disease. In some embodiments, the targeting receptor, e.g., a CAR, is capable of targeting engineered T cells to tissues comprising endothelial cells, e.g., the targeting receptor is a CAR targeting VCAM-1, e.g., for suppressing immune responses, including inflammation, in disorders such as Crohn's disease and multiple sclerosis. In some embodiments, the targeting receptor, e.g., a CAR, is capable of targeting engineered T cells to endothelial cells, e.g., the targeting receptor is a CAR targeting CEACAM6, e.g., for suppressing immune responses in disorders such as Crohn's disease. In some embodiments, the targeting receptor, e.g., a CAR, is capable of targeting engineered T cells to B cells, e.g., the targeting receptor is a CAR targeting CD19, e.g., for suppressing immune responses in disorders such as in multiple sclerosis and systemic lupus erythematosus. In some embodiments, the targeting receptor, e.g., a CAR, is capable of targeting engineered T cells to B cells, e.g., the targeting receptor is a CAR targeting CD20, e.g., for suppressing immune responses in disorders such as in multiple sclerosis and systemic lupus erythematosus. In some embodiments, the targeting receptor, e.g., a CAR, is capable of targeting engineered T cells to an inflammatory tissue, e.g., the targeting receptor is a CAR targeting TNFA, e.g., for suppressing immune responses in disorders such as inflammatory bowel disease, ulcerative colitis, or Crohn's disease. In some embodiments, the targeting receptor, e.g., a CAR is capable of targeting engineered T cells to a neurological tissue, e.g., the targeting receptor is a CAR targeting MBP, MOG, or PLP, e.g., for suppressing immune responses in disorders such as multiple sclerosis. In some embodiments, the targeting receptor, e.g., a CAR, is capable of targeting engineered T cells to tissues comprising mature B lymphocytes, e.g., the targeting receptor is a CAR targeting TNFRSF17, e.g., for suppressing immune responses in disorders such as systemic lupus erythematosus. In some embodiments, the targeting receptor, e.g., a CAR, is capable of targeting engineered T cells to synovial tissue, e.g., the targeting receptor is a CAR targeting citrullinated vimentin e.g., for suppressing immune responses in disorders such as rheumatoid arthritis.

In some embodiments, the targeting receptor is a CAR targeting DPP6, SCL2A2, glutamate decarboxylase (GAD2), demoglein 3 (DSG3), and MHC class I HLA-A (HLA-A*02).

In some embodiments, the methods comprise administering engineered T cells comprising a modification, e.g., knockdown, of an endogenous nucleic acid sequence encoding an IFNG, a modification, e.g., knockdown, of an endogenous nucleic acid sequence encoding a TNFA, insertion of sequence(s) encoding a regulatory T cell promoting molecule, an insertion of sequence(s) encoding a targeting receptor, e.g., a CAR, and further comprising a modification, e.g., knockdown, of TCR sequence(s).

In some embodiments, the sequence(s) to be inserted are inserted into the sequence(s) to be modified, e.g., knocked down, e.g., a CAR sequence is inserted into a TNFA genomic sequence, thereby modifying, e.g., knocking down, the TNFA sequence.

In some embodiments, the methods comprise administering a population of T cells comprising T cells that are engineered as described above. In some embodiments, at least 40%, 45%, preferably at least 50%, 55%, 60%, 65%, 70%, 75%, 80%, 85%, 90%, 95%, or 100% of the T cells in the population of T cells are engineered, e.g., as assessed by sequencing, e.g., NGS.

In some embodiments, the autoimmune disorder is selected from ulcerative colitis, Crohn's disease, rheumatoid arthritis, psoriasis, multiple sclerosis, systemic lupus erythematosus, type 1 diabetes, and graft versus host disease (GvHD). In some embodiments, the engineered T cells have autologous or allogenic use.

In some embodiments, the effectiveness of treatment using the engineered T cell described above can be assessed in an animal model, e.g., mouse model, of graft versus host disease by measuring the animal's weight or survival (wherein the animals are sacrificed after loss of a substantial portion of body weight, e.g., 20% of starting body weight) following administration of the engineered T cell. In some embodiments, effective treatment results in a statistically significant increase in survival rate as compared to a suitable control, e.g., an animal treated with PBMC.

### EXAMPLES

The following examples are provided to illustrate certain disclosed embodiments and are not to be construed as limiting the scope of this disclosure in any way.

### Example 1. General Methods

### 1.1. Preparation of Lipid Nanoparticles

In general, the lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

Unless otherwise specified, the lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:1 by weight, unless otherwise specified.

LNPs were prepared using a cross-flow technique utilizing impinging jet mixing of the lipid in ethanol with two volumes of RNA solutions and one volume of water.

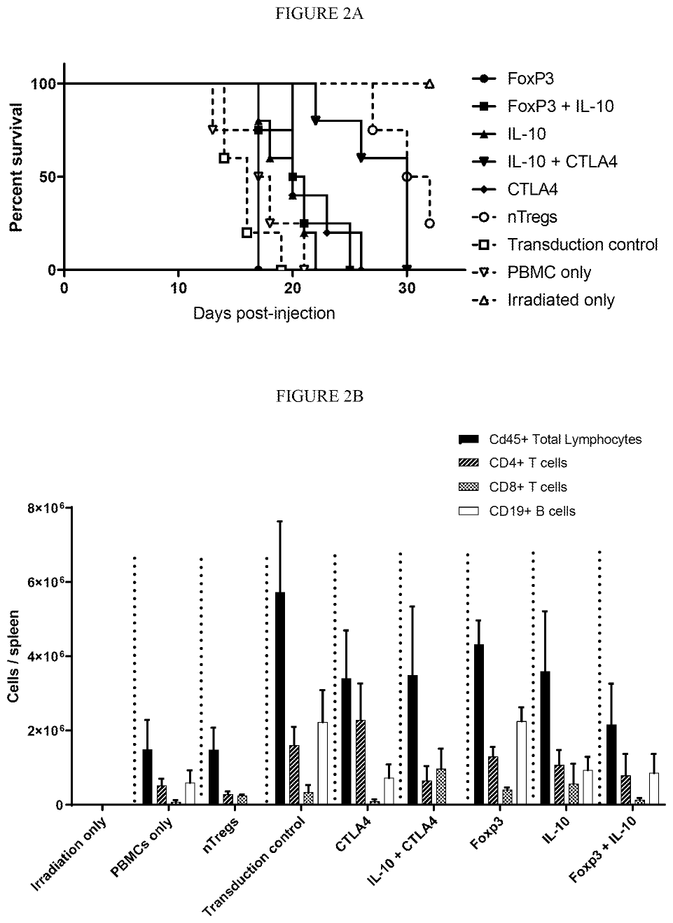

The lipids in ethanol were mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). LNPs were concentrated using tangential flow filtration on a flat sheet cartridge (Sartorius, 100kD MWCO) and buffer exchanged using PD-10 desalting columns (GE) into 50 mM Tris, 45 mM NaCl, 5% (w/v) sucrose, pH 7.5 (TSS). Alternatively, the LNPs were optionally concentrated using 100 kDa Amicon spin filter and buffer exchanged using PD-10 desalting columns (GE) into TSS. The resulting mixture was then filtered using a 0.2 m sterile filter. The final LNP was stored at 4° C. or −80° C. until further use.

### 1.2. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/L linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For IPLC purified mRNA, after the LiCl precipitation and reconstitution, the mRNA was purified by RP-IP IPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcuspyogenes (“Spy”) Cas9 mRNA was generated from plasmid DNA encoding an open reading frame according to the nucleic acid sequences described herein. For the mRNA nucleic acid sequences below, it is understood that Ts should be replaced with Us (which were N1-methyl pseudouridines as described above). Messenger RNAs used in the Examples include a 5′ cap and a 3′ poly-A tail, e.g., up to 100 nts (SEQ ID NO: 146).

### 1.3. Next-Generation Sequencing (“NGS”) and Analysis for On-Target Editing Efficiency

Genomic DNA was extracted using QuickExtract™ DNA Extraction Solution (Lucigen, Cat. QE09050) according to the manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an indel was calculated. The editing percentage (e.g., the “editing efficiency” or “percent editing” or “percent indels”) is defined as the total number of sequence reads with indels over the total number of sequence reads, including wild type.

### Example 2. Suppressive Ability of T Cells Overexpressing Treg Associated Factors

CD3+CD4+ T cells were transduced to overexpress Treg-associated transcription factors Foxp3, Foxp3 with IL10, IL10, CTLA4, and IL10 with CTLA4, and assayed for their ability to suppress inflammatory immune responses.

### 2.1 T Cell Preparation

Human CD3+CD4+ T cells were prepared internally from a fresh leukopak (StemCell Technologies, Donor #RG1953). For internal preparation, CD3+CD4+ T cells were isolated by negative selection using the human CD4+ T cell isolation kit (Miltenyi; Cat no. 130-096-533) following the manufacturer's protocol. The isolated CD3+CD4+ T cells were frozen in Cryostor CS10 freezing media (Cat No. 07930) until further use. On the day of activation, frozen CD3+CD4+ T cells were thawed using a 37° C. water bath. Thawed CD3+CD4+ T cells were plated at a density of 1×10⁶ cells/mL in a total of 5 ml of T cell RPMI media composed of RPMI 1640 (Corning; Cat No. 10-040-CV) containing 10% (v/v) of fetal bovine serum (Gibco; Cat No. A31605-01), 1× Glutamax (Gibco; Cat. 35050-061), 50 μM of 2-Mercaptoethanol (Gibco; 31350-010), 1× non-essential amino acids (Corning, Cat. 25-0250-CI), 1 mM sodium pyruvate (Corning; Cat No. 25-000-CI), 10 mM HEPES buffer (Gibco; Cat No. 15630-080), 1× of Penicillin-Streptomycin (Gibco; Cat No. 15140-122) with added cytokines 100 U/mL of recombinant human interleukin-2 (StemCell Technologies; Cat No. 78036.1), 5 ng/ml recombinant human interleukin 7 (StemCell technologies, Cat No. 78053.1), and 5 ng/ml recombinant human interleukin 15 (StemCell technologies, Cat No. 78031.1). Cells were activated with by addition of 25 uL/mL ImmunoCult Human CD3/CD28/CD2 T cell Activator (Stemcell Technologies, Cat. 10990) and cultured at 37° C. for 48 hours prior to lentiviral transduction.

### 2.2 T Cell Transduction and Cell Sorting

To overexpress IL10, CTLA4, and FOX3P, activated CD3+CD4+ T cells were transduced with lentiviral constructs, either individually or together. Forty-eight hours after activation, CD3+CD4+ T cells were harvested, washed and resuspended at a density of 1×10⁶ cells/100 uL T cell RPMI media. 100 uL of concentrated viral supernatant was added to the CD3+CD4+ T cells and centrifuged at 1000×g for 60 mins at 37° C. Following transduction, the CD3+CD4+ T cells were resuspended in the cell/viral supernatant mixture and transferred to a single well of a 6-well G-rex (Wilson Wolf, Cat. 80240M) containing 20 mL T cell RPMI media supplemented with 500 U/mL IL-2, 5 ng/mL IL-7, and 5 ng/mL IL-17. The transduced CD3+CD4+ T cells were cultured for 4 to 6 days and sorted using a BD FACSAria™ Fusion Cell Sorter (BD Biosciences) to isolate cells expressing the target of interest. Following sorting, the CD3+CD4+ T cells were cultured in 6-well Grex plates with 20 mL T cell RPMI media containing components as mentioned in 2.1 and further supplemented with 500 U/mL IL-2, 5 ng/mL IL7, and 5 ng/mL IL17, until day 25.

Natural regulatory T cells (nTregs) were prepared using methods known in the art. Briefly, autologous PBMCs were thawed and treated to isolate CD4+ T cells using a CD4+ T cell negative selection kit (Miltenyi, Cat. 130-096-533) according to manufacturer's instructions. CD3+CD4+ T cells were resuspended in FACS buffer at 1×10⁷ cells/mL and stained with BV421 anti-CD4 (Biolegend, Cat. 300532), APC anti-CD25 (Biolegend, Cat. 302610) and PE-Cy7 anti-CD127 (Biolegend, Cat. 351320) for 30 mins at 4° C. The top 3-5% highest expressing CD25+ cells from the CD4+CD127− population were sorted by FACS into T cell RPMI media culture containing 50% fetal bovine serum. The sorted CD3+CD4+CD25+CD127− nTregs were plated in a 6-well Grex containing 20 mL T cell RPMI media supplemented with components as mentioned in Example 2.1 further supplemented with 500 U/mL IL-2 (Stemcell Technologies, Cat. 78036.1), 100 nM Rapamycin (Millipore Sigma, Cat. 553211) and 25 μl/mL anti-CD3/28/2 Immunocult T cell Activator (Stemcell Technologies, Cat. 10990). IL-2 (Stemcell Technologies, Cat. 78036.1) and Rapamycin (Millipore Sigma, Cat. 553211) were added every other day for 7 days, at which point only 500 U/ml IL-2 was added (Stemcell Technologies, Cat. 78036.1) every other day. On day 12, nTregs were harvested, washed and plated in 6-well Grex plate in T cell RPMI media supplemented with components as mentioned in A. 1, 500 U/mL IL-2 (Stemcell Technologies, Cat. 78036.1) until the day of injection.

### 2.3 Validating Target Expression

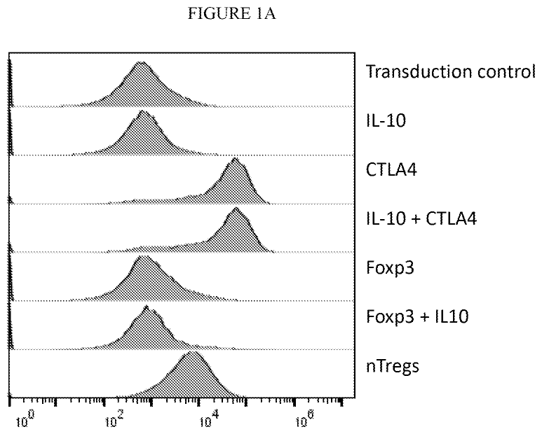

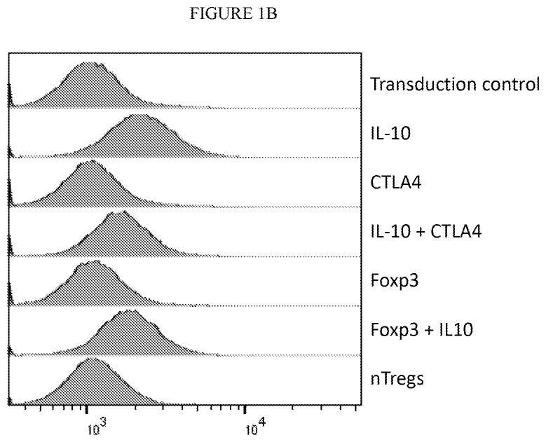

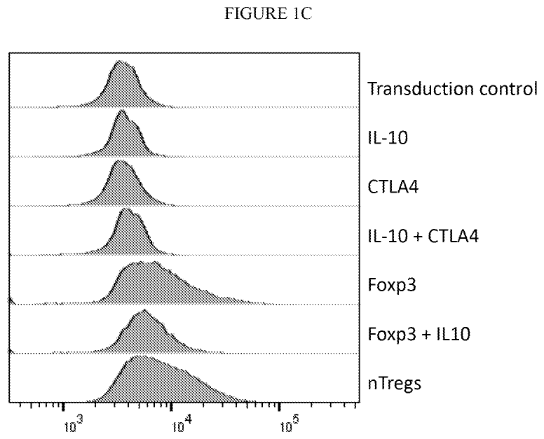

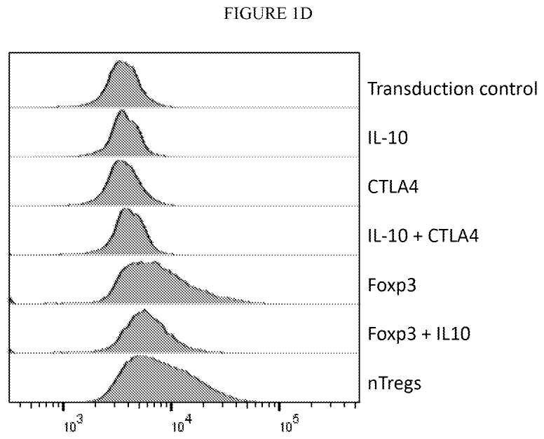

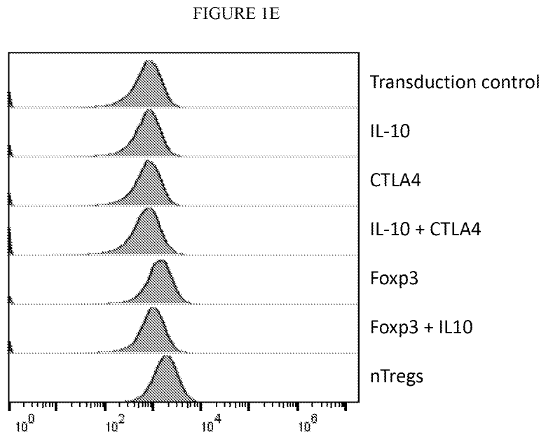

Target expression was verified by flow cytometry. Five-hundred thousand transduced CD3+CD4+ T cells were permeabilized with the FoxP3/Transcription Factor Staining Buffer Set (eBioscience Cat. 00-5523-00), according to manufacturer's instructions. Following permeabilization, the transduced CD3+CD4+ T cells were incubated with a panel of antibodies consisting of either (1) PerCP/Cy 5.5 anti-FoxP3 (BD Biosciences, Cat. 561493), eFluor660 anti-Eos (Invitrogen, Cat. 50-5758-80), Pacific blue anti-Helios (Biolegend, Cat. 137220) or (2) BV421 anti-IL-10 (Biolegend, Cat. 501422) and APC anti-CTLA4 (Biolegend, Cat. 369612). Stained transduced CD3+CD4+ T cells were processed on a Cytoflex flow cytometer (Beckman Coulter) and analyzed using the FlowJo software package. Transduced CD3+CD4+ T cells were gated based on size, shape, prior to quantification of targets in the antibody panel. Overexpression of a target was characterized relative to the transduction control. The desired level of expression for a given target was that equal to, or greater than, nTregs (Table 4 and FIGS. 1A-E). The nTreg sample expressed elevated levels of Foxp3, Helios, and Eos, which correlates with a highly pure and suppressive phenotype.

TABLE 4
Mean fluorescent intensity for protein expression in CD3+CD4+
T cells following lentiviral transduction

| Sample | CTLA4 | IL10 | Foxp3 | Helios | Eos | n |
|---|---|---|---|---|---|---|
| Transduction control | 2203 | 1285 | 3948 | 1521 | 877 | 1 |
| IL10 | 2291 | 2448 | 3945 | 1396 | 871 | 1 |
| CTLA4 | 47928 | 1296 | 3936 | 1334 | 939 | 1 |
| IL10 + CTLA4 | 49948 | 1802 | 4372 | 1304 | 803 | 1 |
| FOXP3 | 3551 | 1347 | 10127 | 1670 | 1704 | 1 |
| FOXP3 + IL10 | 3952 | 2050 | 7339 | 1525 | 2796 | 1 |
| nTregs | 9324 | 1282 | 10047 | 5018 | 2016 | 1 |

### 2.4 In Vivo Assessment of Immunosuppression in a Model of GvHD

The in vivo suppressive function of the sorted transduced CD3+CD4+ T cells was assessed using the graft versus host disease mouse model.

Sorted CD3+CD4+ T cells for in vivo injections were harvested and processed with a dead cell removal kit (Miltenyi, Cat. 130-090-101) according to manufacturer's instructions. Autologous PBMCs were thawed as described above in the Examples. PBMCs were added to each assay population at a 1:1 ratio and cells resuspended in PBS to 6×10⁶/150 μL. The PBMC only group was resuspended at 3×10⁶/150 μL.

Female NOG mice (NOD.Cg-Prkdc^{scid} II2rg^{tm1Sug}/JicTac; Taconic, Cat No. NOG-F) were conditioned for cellular transplant by sublethal irradiation (200 rads) using X-rays (RS-2000 irradiator; Rad Source Technologies) one day before injection. Cohorts of irradiated NOG mice were injected intravenously with 150 μL of each test cell population. Five irradiated mice were not injected and were used as the irradiation only control. Body weight was monitored daily. Upon 20% weight loss, mice were sacrificed and the cellular composition of their spleens was assessed. Survival was plotted to understand the survival rate of mice in each test group. Only T cells transduced with both IL-10 and CTLA4 prolonged survival to levels similar to nTregs, as shown in Table 5 and FIG. 2A.

TABLE 5
Percent survival days after injection of lentiviral transduced CD3+CD4+ cells

| | | | | | | | Transduction | PBMC | Irradiated |
|---|---|---|---|---|---|---|---|---|---|
| | FoxP3 | FoxP3 + IL-10 | IL10 | IL10 + CTLA4 | CTLA4 | nTregs | control | only | only |
| Day | (n = 3) | (n = 4) | (n = 5) | (n = 5) | (n = 5) | (n = 3) | (n = 5) | (n = 4) | (n = 5) |
| 12 | 100% | 100% | 100% | 100% | 100% | 100% | 100% | 100% | 100% |
| 13 | 100% | 100% | 100% | 100% | 100% | 100% | 100% | 75% | 100% |
| 14 | 100% | 100% | 100% | 100% | 100% | 100% | 60% | 75% | 100% |
| 16 | 100% | 100% | 100% | 100% | 100% | 100% | 20% | 75% | 100% |
| 17 | 0% | 75% | 80% | 100% | 100% | 100% | 0% | 50% | 100% |
| 18 | | 75% | 60% | 100% | 100% | 100% | | 25% | 100% |
| 20 | | 50% | 40% | 100% | 40% | 100% | | 25% | 100% |
| 21 | | 25% | 20% | 100% | 40% | 100% | | 0% | 100% |
| 22 | | 0% | 0% | 80% | 40% | 100% | | | 100% |
| 23 | | | | 80% | 20% | 100% | | | 100% |
| 26 | | | | 60% | 0% | 100% | | | 100% |
| 27 | | | | 60% | | 67% | | | 100% |
| 30 | | | | 0% | | 33% | | | 100% |
| 32 | | | | | | 0% | | | 100% |

In order to confirm engraftment of human leukocytes, the splenic composition was assessed. At the time of euthanasia, each animal's spleen was collected in a gentleMACS C tube (Miltenyi, Cat. 130-096-334) containing PBS. The spleens were dissociated using a gentleMACS Octo Dissociator machine (Miltenyi, 130-095-937, program mSpleen01_01). The cell suspension was filtered through a 70-micron cell strainer (Corning, Cat. 08-771-2) and cells were counted using the Vi-CELL XR Cell Viability Analyzer (Beckman Coulter). Approximately one million viable splenocytes were resuspended in FACS buffer and stained for 30 min at 4° C. with panel of antibodies consisting of anti-human CD3 (Alexa Fluor 488 (Biolegend, Cat. 317310 or PerCP/Cyanine 5.5 (Biolegend, Cat. 300327))), BV650 anti-human CD19 (Biolegend, Cat. 302238), APC anti-human CD45 (BD Pharmigen, Cat. 561864), APC-Fire 750 anti-human CD4 (Biolegend, Cat. 300560) and BV421 anti-mouse Ter 19 (Biolegend, Cat. 116234). The splenocytes were washed with FACS buffer, processed on a Cytoflex flow cytometer (Beckman Coulter) and analyzed using the FlowJo software package. CD4 T cells were defined as Ter119−CD45+CD19−CD3+CD4+. CD8 T cells were defined as Ter119−CD45+CD19−CD3+CD4−. B cells were defined as Terl19−CD45+CD19+CD3−. In order to determine the number of cells of individual populations, the percentage of each population was applied to the total number of splenocytes recovered. Mice treated with T cells transduced with lentiviral vectors to induce overexpression both IL10 and CTLA4, and nTregs had lower B cell percentages and numbers than untreated PBMC mice. Data shown in Table 6 and FIG. 2B.

TABLE 6
Quantification of human lymphocytes in spleens of mice

| | Total Human Lymphocytes | | | | | | | | | |
|---|---|---|---|---|---|---|---|---|---|---|
| | (CD45+) × 10\hat{ }5 | | | Human CD4 T cells × 10\hat{ }5 | | Human CD8 T cells × 10\hat{ }5 | | Human B cells × 10\hat{ }5 | | |
| Sample | Mean | SEM | n | Mean | SEM | Mean | SEM | Mean | SEM | n |
| PBMCs only | 14.93 | 7.91 | 4 | 5.15 | 1.86 | 0.73 | 0.49 | 5.90 | 3.37 | 4 |
| Transduction | 57.27 | 19.09 | 5 | 16.00 | 5.00 | 3.35 | 1.94 | 22.25 | 8.65 | 5 |
| control | | | | | | | | | | |
| nTregs | 14.80 | 5.96 | 4 | 2.82 | 0.74 | 2.44 | 0.29 | 0.01 | 0.01 | 4 |
| IL10 | 35.90 | 16.23 | 5 | 10.69 | 4.05 | 5.64 | 5.39 | 9.30 | 3.59 | 5 |
| CTLA4 | 34.08 | 12.90 | 5 | 22.83 | 9.83 | 0.86 | 0.62 | 7.23 | 3.61 | 5 |
| IL10 + CTLA4 | 34.86 | 18.52 | 5 | 6.47 | 3.98 | 9.66 | 5.45 | 0.02 | 0.01 | 5 |
| Foxp3 | 41.90 | 7.77 | 3 | 13.00 | 2.59 | 4.01 | 0.68 | 22.50 | 3.74 | 4 |
| Foxp3 + IL10 | 21.57 | 11.06 | 4 | 7.87 | 5.81 | 1.32 | 0.51 | 8.56 | 5.11 | 4 |

### 2.5 Cytokine Profile Analysis of Transduced CD3+CD4+ T Cells

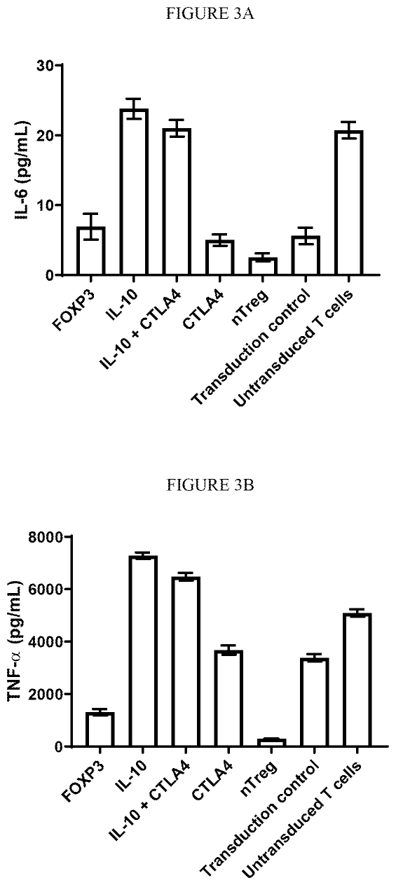

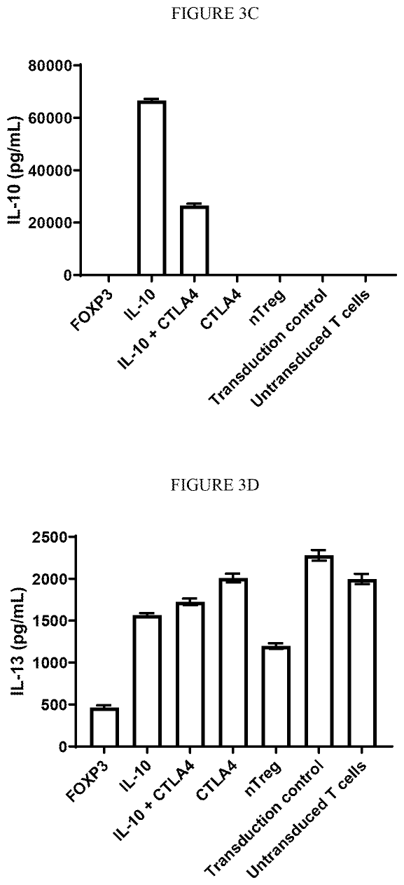

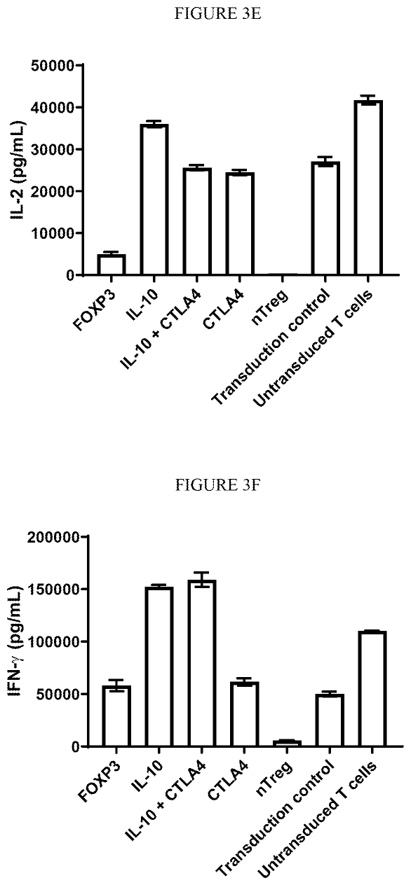

Sorted transduced CD3+CD4+ T cells were stimulated to assess their cytokine profile. Sorted transduced CD3+CD4+ T cells were plated at 1×10\hat{ }5 T cells/well, in a U-bottom culture plate, in a total of 200 μL T cell RPMI media with or without 25 uL/mL ImmunoCult Human CD3/CD28/CD2 T cell Activator (Stemcell Technologies, Cat. 10990) and cultured at 37° C. for 48 hours. Following 48 hours of culture, the culture plate was centrifuged, the supernatants collected and frozen for subsequent cytokine quantification using a custom U-PLEX Biomarker kit (Meso Scale Diagnostics, Cat. K15067L-2), according to manufacturer's instructions. Specifically, the U-PLEX Biomarker kit was used to quantify the following human cytokines: IFNG, TNFA, IL6, IL2, IL13, and IL10. The U-PLEX Biomarker plates were read using the Meso Quickplex SQ120 instrument (Meso Scale Discovery) and the data were analyzed with the Discovery Workbench 4.0 software package (Meso Scale Discovery). Results are shown in Table 7 and FIGS. 3A-3F. CD3+CD4+ T cells transduced with a lentiviral expression vector with a sequence encoding IL10 secreted large quantities of IL10 upon TCR stimulation. Overexpression of IL10 also increased secretion of IL6, IFNG, and TNFA. In contrast, T cells transduced with a lentiviral expression vector with a sequence encoding FoxP3 displayed reduced expression of all quantified cytokines. Natural Tregs also displayed reduced secretion of quantified cytokines, which is characteristic of highly pure and suppressive nTregs.

TABLE 7
In vitro cytokine production (pg/ml) of transduced cells upon cell stimulation

| | IFNG | | IL10 | | IL13 | | IL2 | | IL6 | | TNFA | | |
|---|---|---|---|---|---|---|---|---|---|---|---|---|---|
| Sample | Mean | SEM | Mean | SEM | Mean | SEM | Mean | SEM | Mean | SEM | Mean | SEM | n |
| Untransduced | 110,200 | 431 | 42 | 3 | 1,997 | 61 | 41,741 | 1,053 | 21 | 1 | 5,092 | 143 | 3 |
| CD3+CD4+ | | | | | | | | | | | | | |
| T cells | | | | | | | | | | | | | |
| Transduction | 49,896 | 2,310 | 29 | 2 | 2,279 | 64 | 27,081 | 1,060 | 6 | 1 | 3,383 | 139 | 3 |
| control | | | | | | | | | | | | | |
| Foxp3 | 57,997 | 5,483 | 5 | 0 | 463 | 27 | 4,978 | 550 | 7 | 2 | 1,306 | 123 | 3 |
| IL10 | 152,243 | 1,803 | 66,549 | 698 | 1,564 | 26 | 35,979 | 747 | 24 | 1 | 7,279 | 123 | 3 |
| CTLA4 | 61,526 | 3,636 | 34 | 0 | 2,008 | 53 | 24,469 | 629 | 5 | 1 | 3,679 | 179 | 3 |
| IL10 + CTLA4 | 159,139 | 6,838 | 26,523 | 737 | 1,725 | 39 | 25,621 | 620 | 21 | 1 | 6,484 | 148 | 3 |
| nTreg | 5,805 | 229 | 24 | 3 | 1,198 | 33 | 384 | 7 | 3 | 1 | 297 | 5 | 3 |

### 2.6 Mixed Lymphocyte Reaction Assay of Suppressive Function

A mixed lymphocyte reaction (MLR) was used to assay the suppressive function of sorted transduced CD3+CD4+ T cells. The MLR is an inflammatory reaction caused by T cells recognizing allogenic leukocytes of another donor as foreign. Tregs are able to suppress this inflammatory reaction. Therefore, the MLR is a standard assay to assess the suppressive capacity of Tregs, including engineered Tregs. If a Treg is suppressive, there is less proliferation and production of inflammatory cytokines by the responding inflammatory T cells.

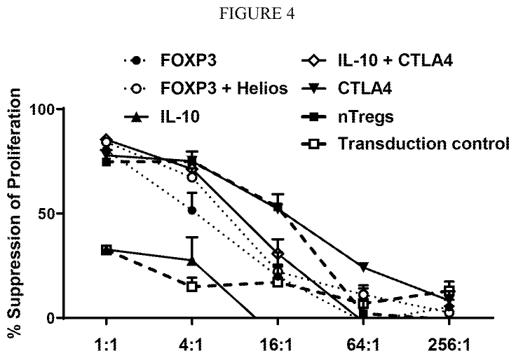

The MLR was conducted in a 96-well U-bottom plate using T cell RPMI media. Untransduced CD3+CD4+ T cells were labelled with CellTrace Violet (CTV) (Thermofisher Scientific; Cat No. C34557) according to manufacturer's instructions and were used as the responding cells. CD3-depleted PBMC from an allogenic donor than used for the transduced T cells were processed using a Dead cell removal kit (Miltenyi; Cat No. 130-090-101) according to manufacturer's instructions. Cultures were prepared by combining 50,000 CTV-labelled T cells, 50,000 CD3-depleted PBMC, and approximately 50,000 (1 to 1), 16,666 (4 to 1), 5,555 (16 to 1), 1,851 (64 to 1) or 617 (256 to 1) sorted transduced CD3+CD4+ T cells per well. Following 5 days of culture at 37° C., the culture plate was centrifuged, and culture supernatants were harvested for cytokine quantification. The cell pellet was resuspended in FACS buffer containing APC/Fire 750 anti-CD4 and placed at 4° C. for 30 mins. The cells were subsequently washed, processed on a CytoFlex flow cytometer (Beckman Coulter), and analyzed using the FlowJo software package. Cells were first gated by positive CD4 expression, followed by CTV expression and finally on the undiluted CTV population. Suppression of CTV-dilution was calculated using the following formula:

$$( log 2 ( y ) of CTV T cells - log 2 ( y ) of CTV T cells with Treg ) ) \text{} / log 2 ( y ) of CTV T cells * 100$$

Where y=mean fluorescent intensity of the entire CTV-labelled population/mean fluorescent intensity of the undiluted portion of the CTV-labelled population. Data is shown in Table 8 and FIG. 4.

TABLE 8
Percent suppression of cell proliferation by transduced
T cells as measured by CTV dilution

| | 1 to 1 | | 4 to 1 | | 16 to 1 | | 64 to 1 | | 256 to 1 | |
|---|---|---|---|---|---|---|---|---|---|---|
| Sample | Mean | SEM | Mean | SEM | Mean | SEM | Mean | SEM | Mean | SEM |
| Transduction | 32.52 | 0.19 | 14.88 | 4.30 | 17.02 | 7.56 | 6.84 | 7.30 | 12.99 | 4.36 |
| control | | | | | | | | | | |
| Foxp3 | 80.34 | 1.10 | 51.50 | 8.41 | 19.91 | 5.59 | −1.95 | 10.75 | 5.24 | 3.50 |
| IL10 | 32.73 | 0.97 | 27.57 | 11.08 | −9.69 | 6.50 | −19.10 | 9.28 | −9.67 | 2.66 |
| CTLA4 | 77.81 | 2.54 | 75.03 | 4.61 | 52.11 | 7.06 | 24.09 | 2.17 | 8.28 | 4.43 |
| IL10/CTLA4 | 85.36 | 1.79 | 71.27 | 1.86 | 30.72 | 6.94 | −1.98 | 2.68 | −7.75 | 2.27 |
| nTreg | 74.68 | 0.76 | 74.89 | 1.84 | 53.10 | 1.84 | 2.01 | 6.18 | −0.87 | 3.07 |

### Example 3. Suppressive Ability of Engineered T Cells

As CD3+CD4+ T cells transduced with lentiviral vectors to promote overexpression of IL-10 and CTLA-4 demonstrated an increase in the production of IFNG and TNFA, these cells were further engineered to disrupt the genes encoding IFNG and TNFA. The suppressive ability of these cells was assessed in vitro and in vivo.

### 3.1 T Cell Engineering

Human CD3+CD4+ T cells were isolated from a leukopak, activated, and transduced with lentivirus constructs to promote overexpression of IL10 and CTLA4 as described in Example 2.2. One day after transduction, the transduced cells were engineered using Cas9 to disrupt the TNFA and IFNG genes. LNPs containing Cas9 mRNA and a sgRNA targeting IFNG (G019753; IFNG guide sequence CCAGAGCAUCCAAAAGAGUG (SEQ ID NO: 14)) or TNFA (G019757; TNFA guide sequence AGAGCUCUUACCUACAACAU (SEQ ID NO: 58)) were formulated as described in Example 1.

G019753:
(SEQ ID NO: 119)
mC*mC*mA*GAGCAUCCAAAAGAGUGGUUUUAGAmGmCmUmAmGmAmAmAmU
mAmGmCAAGUUAAAAUAAGGCUAGUCCGUUAUCAmAmCmUmUmGmAmAmAm
AmAmGmUmGmGmCmAmCmCmGmAmGmUmCmGmGmUmGmCmU*mU*mU*mU
G019757:
(SEQ ID NO: 120)
mA*mG*mA*GCUCUUACCUACAACAUGUUUUAGAmGmCmUmAmGmAmAmAmU
mAmGmCAAGUUAAAAUAAGGCUAGUCCGUUAUCAmAmCmUmUmGmAmAmAm
AmAmGmUmGmGmCmAmCmCmGmAmGmUmCmGmGmUmGmCmU*mU*mU*mU

Each LNP preparation was incubated in OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512), 200 U/mL recombinant human interleukin-2, 5 ng/ml recombinant human interleukin 7, and 5 ng/ml recombinant human interleukin-15 supplemented with 10 ug/ml recombinant human ApoE3 (Peprotech, Cat. 350-02) for 15 minutes at 37° C. Forty-eight hours post-activation, the transduced T cells were washed and suspended in OpTmizer base media with 200 U/mL recombinant human interleukin-2, 5 ng/ml recombinant human interleukin 7, and 5 ng/ml recombinant human interleukin-15, along with 2.5% human AB serum (Gemini, Cat. 100-512). Pre-incubated LNP mix was added to the each 15 mL tube to yield a final concentration of 5 ug/ml total RNA, in groups with double knockout final concentration was 10 ug/ml of total RNA. Media supplemented with ApoE3 was used as a vehicle control. After 24 hours, T cells were collected, washed, and cultured in T cell RPMI media with cytokines as described in Example 2, for expansion until day of injection (Day 15 post-activation).

### 3.2 Flow Cytometry Analysis of Protein Expression in Engineered CD3+CD4+ T Cells

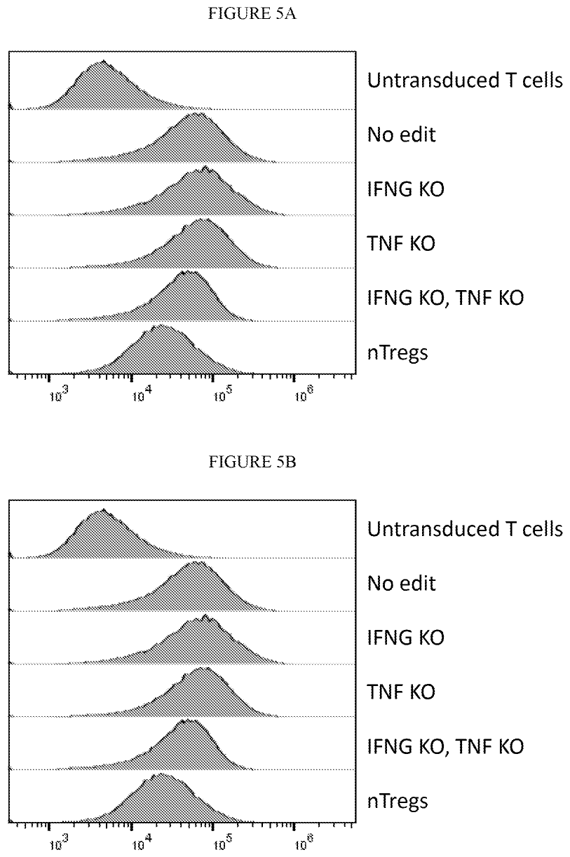

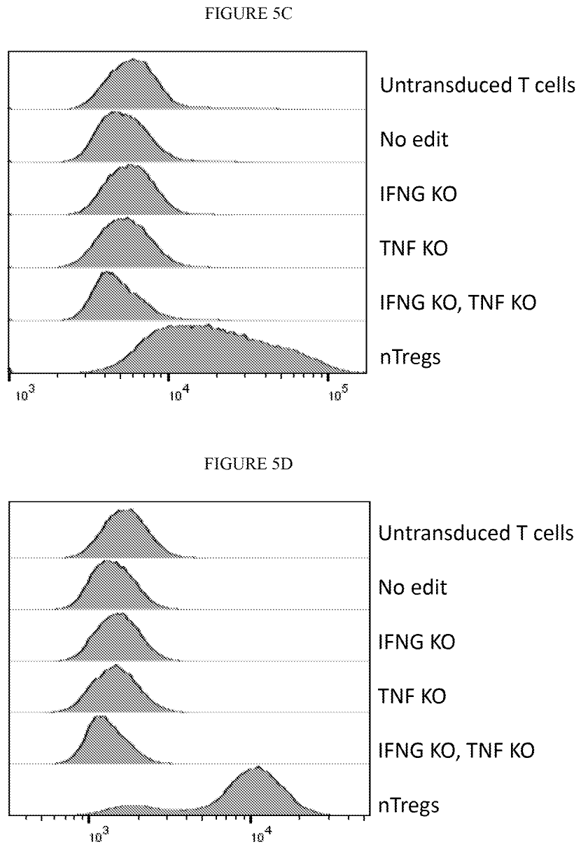

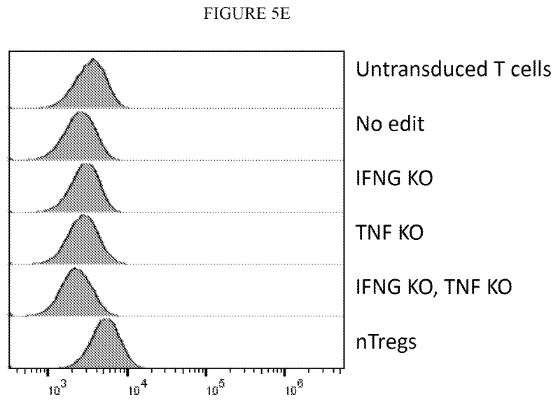

Target expression was verified by flow cytometry as in Example 2. Data are shown in Table 9 and FIGS. 5A-5E.

TABLE 9
Mean fluorescent intensity of CD3+CD4+ cells following T cell engineering

| | IL10 | | CTLA4 | | Foxp3 | | Helios | | Eos | | |
|---|---|---|---|---|---|---|---|---|---|---|---|
| Sample | Mean | SEM | Mean | SEM | Mean | SEM | Mean | SEM | Mean | SEM | n |
| Untransduced | 1613.5 | 2.5 | 8069.5 | 164.4 | 7650.0 | 94.0 | 1866.0 | 11.0 | 3559.5 | 13.5 | 2 |
| CD3+CD4+ | | | | | | | | | | | |
| T cells | | | | | | | | | | | |
| MOCK KO | 2868.0 | 57.0 | 71630.0 | 553.0 | 6134.5 | 0.5 | 1475.0 | 15.0 | 2666.5 | 100.5 | 2 |
| IFNG KO | 3073.5 | 132.5 | 95616.0 | 3234.3 | 6468.5 | 39.5 | 1623.5 | 36.5 | 3145.0 | 118.0 | 2 |
| TNF KO | 2734.0 | 106.0 | 85600.5 | 1232.1 | 6363.5 | 393.5 | 1638.5 | 97.5 | 3344.0 | 409.0 | 2 |
| IFNG KO, | 2190.5 | 75.5 | 48940.5 | 1751.9 | 5316.5 | 191.5 | 1321.5 | 61.5 | 2406.0 | 18.0 | 2 |
| TNF KO | | | | | | | | | | | |
| nTregs | 1467.5 | 102.5 | 38990.0 | 1857.6 | 24685.0 | 866.0 | 8822.5 | 645.5 | 5494.5 | 48.5 | 2 |

### 3.3 In Vivo Assessment of Inflammatory Response in a GvHD Model

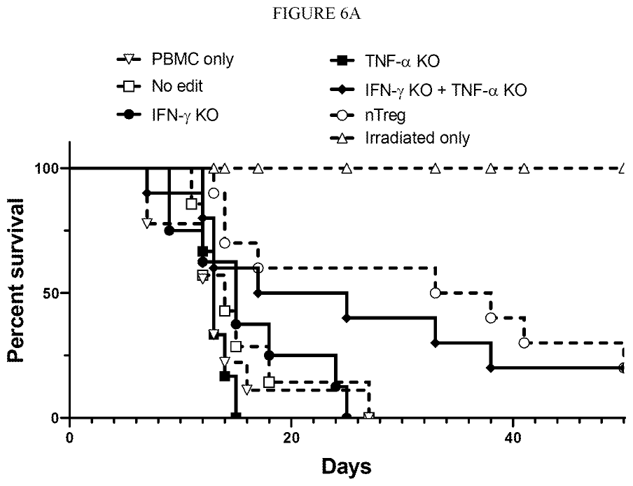

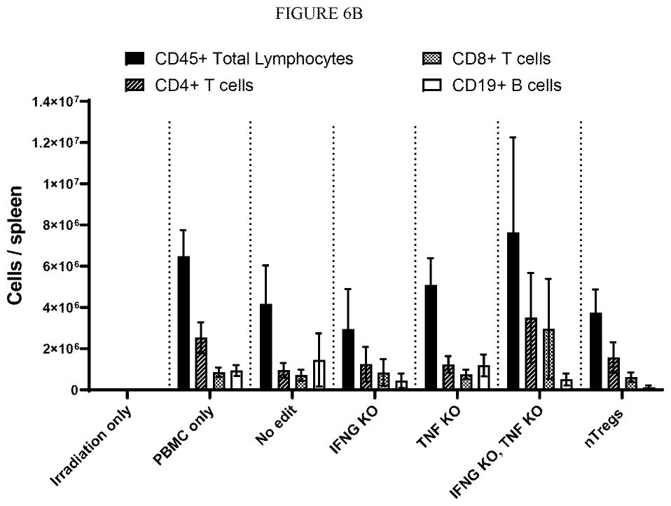

The in vivo suppressive function of engineered CD3+CD4+ T cells overexpressing IL10 and CTLA4 alone (Mock KG), or in combination with editing to knockdown expression of IFNG, TNFA, or a double knockdown of IFNG and TNFA was assessed using the graft versus host disease mouse model as in Example 2. Survival data are shown in Table 10 and FIG. 6A. Human leukocyte engraftment was assessed through the splenic composition as in Example 2. Data are shown in Table 11 and FIG. 6B.

TABLE 10
Percent survival days after injection of engineered CD3+CD4+ cells

| | | | | IFNG KO | | | |
|---|---|---|---|---|---|---|---|
| | MOCK KO | IFNG KO | TNF KO | TNF KO | nTregs | | |
| | (n = 7) | (n = 8) | (n = 6) | (n = 10) | (n = 10) | PBMC | Irradiated |
| Days post | P-value vs. PBMC | | | | | only | only |
| injection | 0.5566 | 0.6053 | 0.7746 | 0.0312 | 0.0012 | (n = 9) | (n = 5) |
| 6 | 100% | 100% | 100% | 100% | 100% | 100% | 100% |
| 8 | 100% | 100% | 100% | 88% | 100% | 78% | 100% |
| 10 | 100% | 75% | 100% | 88% | 100% | 78% | 100% |
| 12 | 58% | 63% | 67% | 75% | 100% | 56% | 100% |
| 14 | 43% | 63% | 17% | 50% | 63% | 34% | 100% |
| 16 | 29% | 38% | 0% | 50% | 63% | 23% | 100% |
| 18 | 15% | 25% | | 38% | 50% | 23% | 100% |
| 20 | 15% | 25% | | 28% | 50% | 23% | 100% |
| 25 | 15% | 0% | | 25% | 50% | 23% | 100% |
| 27 | 0% | | | 25% | 50% | 0% | 100% |
| 33 | | | | 13% | 38% | | 100% |
| 38 | | | | 0% | 25% | | 100% |
| 41 | | | | | 13% | | 100% |
| 50 | | | | | 0% | | 100% |

TABLE 11
Quantification of human lymphocytes in spleens of mice

| | Total Human Lymphocytes | | | | | | | | | |
|---|---|---|---|---|---|---|---|---|---|---|
| | (CD45+) × 10\hat{ }5 | | | Human CD4 T cells × 10\hat{ }5 | | Human CD8 T cells × 10\hat{ }5 | | Human B cells × 10\hat{ }5 | | |
| Sample | Mean | SEM | n | Mean | SEM | Mean | SEM | Mean | SEM | n |
| PBMC only | 64.70 | 11.98 | 8 | 25.30 | 7.02 | 8.56 | 2.19 | 9.35 | 2.52 | 9 |
| MOCK KO | 41.72 | 18.66 | 6 | 9.48 | 3.57 | 7.10 | 2.77 | 14.49 | 12.88 | 6 |
| IFNG KO | 29.40 | 19.62 | 6 | 12.48 | 8.37 | 8.41 | 6.52 | 4.41 | 3.53 | 6 |
| TNF KO | 50.82 | 11.97 | 6 | 12.32 | 4.39 | 7.49 | 2.57 | 11.86 | 5.80 | 5 |
| IFNG KO, | 76.40 | 46.06 | 8 | 35.00 | 21.81 | 29.62 | 24.24 | 5.08 | 2.91 | 8 |
| TNF KO | | | | | | | | | | |
| n Tregs | 37.40 | 11.33 | 7 | 15.66 | 7.91 | 6.16 | 2.41 | 1.25 | 0.96 | 7 |

### 3.4 Cytokine Profile of CD3+CD4+ Engineered Cells

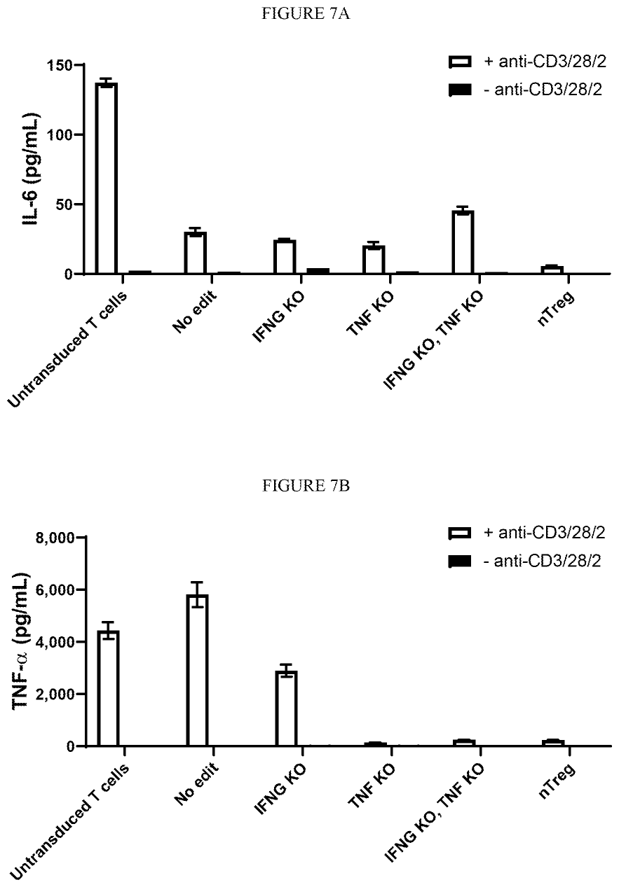

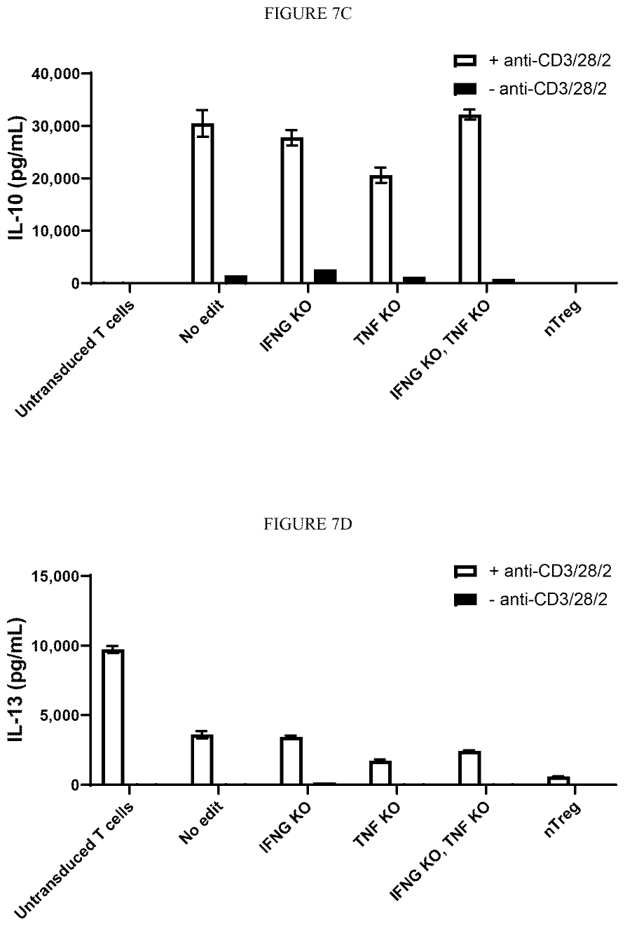

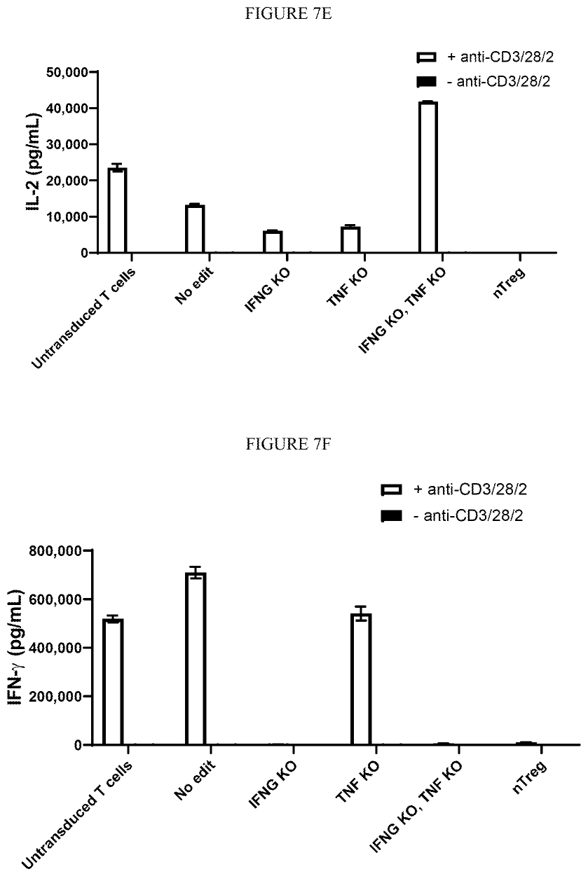

Sorted transduced CD3+CD4+ T cells were stimulated to assess their cytokine profile in triplicate as in Example 2. Results are shown in Table 12 and FIGS. 7A-7F.

TABLE 12
In vitro cytokine production (pg/ml) of engineered T cells upon cell stimulation

| | Untransduced | | | | | | | | IFNG KO | | | |
|---|---|---|---|---|---|---|---|---|---|---|---|---|
| | T cells | | No Edit | | IFNG KO | | TNF KO | | TNF KO | | nTregs | |
| Sample | Mean | SEM | Mean | SEM | Mean | SEM | Mean | SEM | Mean | SEM | Mean | SEM |
| IFNG | 519,150 | 13,984 | 709,650 | 24,157 | 2,091 | 172 | 540,921 | 28,764 | 5,413 | 213 | 10,292 | 294 |
| IL10 | 77 | 3 | 30,500 | 2,557 | 27,767 | 1,484 | 20,622 | 1,467 | 32,167 | 967 | 12 | 1 |
| IL13 | 9,714 | 254 | 3,608 | 263 | 3,429 | 113 | 1,735 | 101 | 2,428 | 41 | 576 | 29 |
| IL2 | 23,535 | 1,088 | 13,211 | 291 | 6,080 | 131 | 7,180 | 467 | 41,787 | 138 | 36 | 3 |
| IL6 | 137 | 3 | 30 | 3 | 24 | 1 | 20 | 3 | 46 | 3 | 5 | 1 |
| TNFA | 4,433 | 326 | 5,810 | 473 | 2,900 | 229 | 138 | 6 | 248 | 3 | 232 | 8 |

### 3.5 Mixed Lymphocyte Reaction Assay of Suppressive Function

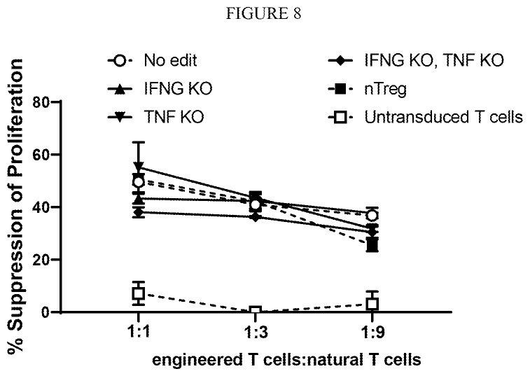

A mixed lymphocyte reaction was used to assay the suppressive function of transduced cells as in Example 2 with the ratios of CTV-labelled T cells to engineered T cells described in Table 13. Data is shown in Table 13 and FIG. 8.

TABLE 13
Percent suppression of cell proliferation by engineered
T cells as measured by CTV dilution

| | 1 to 1 | | 1 to 3 | | 1 to 9 | |
|---|---|---|---|---|---|---|
| Sample | Mean | SEM | Mean | SEM | Mean | SEM |
| Untransduced T cells | 7.17 | −4.32 | −6.91 | 4.97 | 3.13 | 4.78 |
| MOCK edit | 49.60 | −0.38 | 40.93 | 2.46 | 36.83 | 1.07 |
| IFNG KO | 43.27 | −1.87 | 42.33 | 3.48 | 37.77 | 2.03 |
| TNF KO | 55.17 | −9.52 | 43.53 | 1.96 | 31.87 | 1.30 |
| IFNG KO, TNFA KO | 38.07 | −1.91 | 36.30 | 0.36 | 30.50 | 2.12 |
| n Tregs | 50.57 | −2.03 | 42.23 | 2.72 | 25.50 | 2.28 |

### Example 4. In Vivo Assessment of Inflammatory Response in a GvHD Model

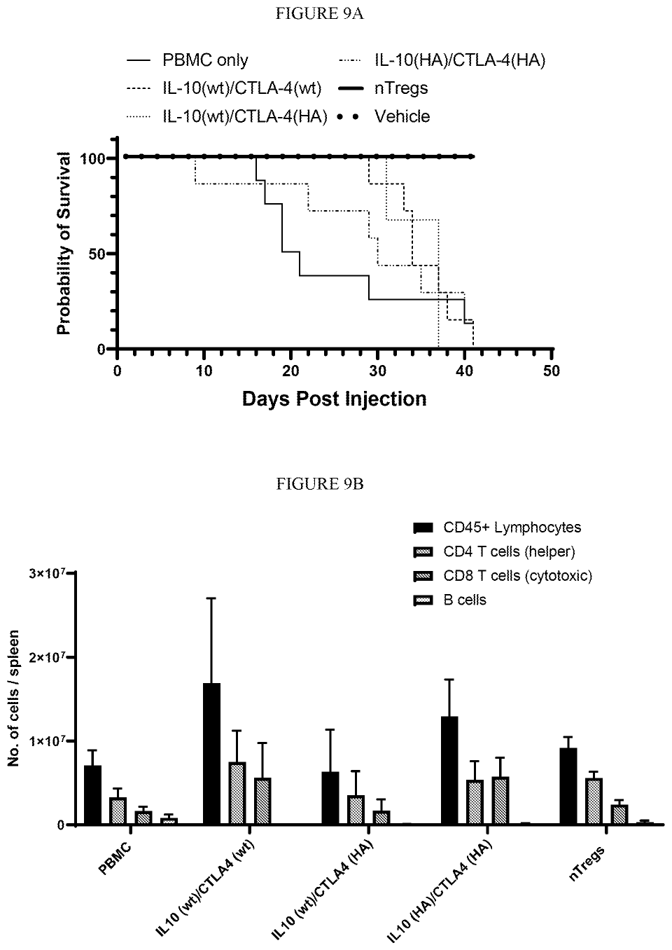

The in vivo suppressive function of engineered CD3+CD4+ T cells overexpressing wild-type or high-affinity versions of IL10 and CTLA4, with editing to knockdown expression of IFNG, TNFA, was assessed using the graft versus host disease mouse model as in Example 2, except that 5×10≢PBMC and Tregs were injected. Survival data are shown in Table 14 and FIG. 9A. Human leukocyte engraftment was assessed through the splenic composition as in Example 2. Data are shown in Table 15 and FIG. 9B.

TABLE 14
Percent survival days after injection of engineered CD3+CD4+ cells

| | IL10 (wt)/ | IL10 (wt)/ | IL10 (HA)/ | | | |
|---|---|---|---|---|---|---|
| | CTLA4 | CTLA4 | CTLA4 | | | |
| | (wt) | (HA) | (HA) | nTregs | | |
| | (n = 7) | (n = 3) | (n = 7) | (n = 7) | | |
| Days post | P-value vs. PBMC | | | | PBMC | Vehicle |
| injection | 0.088 | 0.25 | 0.32 | 0.0008 | (n = 8) | (n = 9) |
| 8 | 100% | 100% | 86% | 100% | 100% | 100% |
| 15 | 100% | 100% | 86% | 100% | 88% | 100% |
| 16 | 100% | 100% | 86% | 100% | 75% | 100% |
| 18 | 100% | 100% | 86% | 100% | 50% | 100% |
| 20 | 100% | 100% | 86% | 100% | 38% | 100% |
| 21 | 100% | 100% | 71% | 100% | 38% | 100% |
| 28 | 85% | 100% | 57% | 100% | 25% | 100% |
| 29 | 85% | 100% | 42% | 100% | 25% | 100% |
| 30 | 85% | 67% | 42% | 100% | 25% | 100% |
| 32 | 71% | 67% | 42% | 100% | 25% | 100% |
| 33 | 42% | 67% | 42% | 100% | 25% | 100% |
| 34 | 42% | 67% | 29% | 100% | 25% | 100% |
| 36 | 29% | 0% | 29% | 100% | 25% | 100% |
| 37 | 29% | | 29% | 100% | 25% | 100% |
| 39 | 29% | | 14% | 100% | 13% | 100% |
| 40 | 0% | | 14% | 100% | 13% | 100% |

TABLE 15
Quantification of human lymphocytes in spleens of mice

| | Total Human Lymphocytes | | | | | | | | |
|---|---|---|---|---|---|---|---|---|---|
| | (CD45+) × 10\hat{ }6 | | | Human CD4 T cells × 10\hat{ }6 | | Human CD8 T cells × 10\hat{ }6 | | Human B cells × 10\hat{ }5 | |
| Sample | Mean | SEM | n | Mean | SEM | Mean | SEM | Mean | SEM |
| IL10(wt)/CTLA4 (wt) | 16.8 | 10.1 | 6 | 7.5 | 3.7 | 5.6 | 4.1 | 0.3 | 0.1 |
| IL10(wt)/CTLA4(HA) | 6.3 | 5 | 3 | 3.5 | 2.9 | 1.7 | 1.4 | 0.5 | 0.5 |
| IL10(HA)/CTLA4(HA) | 12.9 | 4.4 | 6 | 5.4 | 2.2 | 5.7 | 2.3 | 1.4 | 0.7 |
| nTreg | 9.2 | 1.3 | 8 | 5.6 | 0.8 | 2.4 | 0.5 | 3 | 2.1 |
| PBMC | 7 | 1.8 | 7 | 3.2 | 1.1 | 1.6 | 0.6 | 8 | 4.2 |

### Example 5. Suppressive Ability of Engineered T Cells Following Exposure to Inflammatory Cytokines

The stability of a Treg suppressive phenotype in an inflammatory environment is a key consideration for a Treg therapy. We therefore exposed sTregs to the inflammatory cytokines TNF-α, TL-6, and IL-1β, and assessed their suppressive function in vitro.

### 5.1 T Cell Engineering

Human CD3+CD4+ T cells were isolated from a leukopak, activated, and transduced with lentivirus constructs to promote overexpression of IL10 and CTLA4 as described in Example 2.2. One day after transduction, the transduced cells were engineered using Cas9 to disrupt the TNFA and IFNG genes as described in Example 3.1 using gRNA G019754 (INFG) and G019760 (TNFA).

A portion of sorted engineered CD3+CD4+ T cells were cultured in the presence of a mixture of inflammatory cytokines, 100 ng/mL each of TNF-αL (Miltenyi; 130-094-014), IL-1β (Miltenyi; 130-093-898), and IL-6 (Miltenyi; 130-095-365). TNF-α, TL-1β, and IL-6 were replenished every two days for eight days, at which point their function was assessed using the MLR suppression assay.

Target expression was verified by flow cytometry as in Example 2. Data are shown in Table 16.

TABLE 16
Mean fluorescent intensity of CD3+CD4+
cells following T cell engineering

| Sample | CTLA4 (mean) |
|---|---|
| Untransduced | 3866 |
| No Edit | 114290 |
| IFNG KO | 114271 |
| TNFA KO | 112277 |
| IFNG/TNFA KO | 116005 |
| No Edit, preconditioned | 78579 |
| IFNG KO, preconditioned | 93574 |
| TNFA KO, preconditioned | 82986 |
| IFNG/TNFA KO, preconditioned | 85807 |

Sorted transduced CD3+CD4+ T cells were stimulated to assess their cytokine profile, in triplicate, as in Example 2. Data are shown in Tables 17 and 18.

TABLE 17
In vitro cytokine production (pg/ml) of engineered T cells upon cell stimulation

| | Untransduced | | No edit | | IFNG KO | | TNF KO | | IFNG KO TNF KO | |
|---|---|---|---|---|---|---|---|---|---|---|
| Sample | Mean | SEM | Mean | SEM | Mean | SEM | Mean | SEM | Mean | SEM |
| IFNG | 73975.5 | 3610.3 | 114184 | 859.7 | 621.2 | 58.1 | 113817.1 | 3318.9 | 711.7 | 120.8 |
| TNFA | 676.8 | 57.7 | 1427.1 | 174 | 880 | 133.5 | 164.7 | 8.7 | 131.6 | 14.7 |
| IL10 | 575.1 | 59.8 | 45237.9 | 2672.3 | 28551.8 | 2639.2 | 38242.1 | 2746.2 | 34482.1 | 3807.2 |
| IL13 | 2791.1 | 109.5 | 1960.8 | 80 | 1487.8 | 137.9 | 1698.8 | 69 | 1703.5 | 91.7 |
| IL2 | 62.9 | 2.1 | 254.2 | 27.6 | 120.2 | 15 | 251 | 20.7 | 172.9 | 17.5 |
| IL6 | 16.7 | 1.6 | 444.2 | 108.4 | 30.2 | 4.7 | 45.9 | 3 | 33.7 | 5.4 |

TABLE 18
In vitro cytokine production (pg/ml) of engineered T cells
exposed to inflammatory cytokines upon cell stimulation

| | No edit | | IFNG KO | | TNF KO | | IFNG KO TNF KO | |
|---|---|---|---|---|---|---|---|---|
| Sample | Mean | SEM | Mean | SEM | Mean | SEM | Mean | SEM |
| IFNG | 315882 | 7526.6 | 595 | 41.9 | 315422 | 1119.8 | 1033.2 | 29.5 |
| TNFA | 2284.5 | 115.9 | 1573.2 | 48.1 | 142.4 | 8.9 | 102.1 | 8.5 |
| IL10 | 28998.4 | 1493.6 | 24158.7 | 1010.4 | 26235.9 | 523.5 | 21074 | 1190.8 |
| IL13 | 3980.6 | 122.8 | 2731.7 | 97.6 | 2654.3 | 63.9 | 2523.7 | 87.3 |
| IL2 | 1078.6 | 80.3 | 93.8 | 6.2 | 428 | 24.3 | 242.2 | 23.1 |
| IL6 | 53.6 | 2.8 | 29.7 | 4.4 | 47.5 | 1.9 | 25.2 | 3.1 |

### 5.2 Mixed Lymphocyte Reaction Assay of Suppressive Function

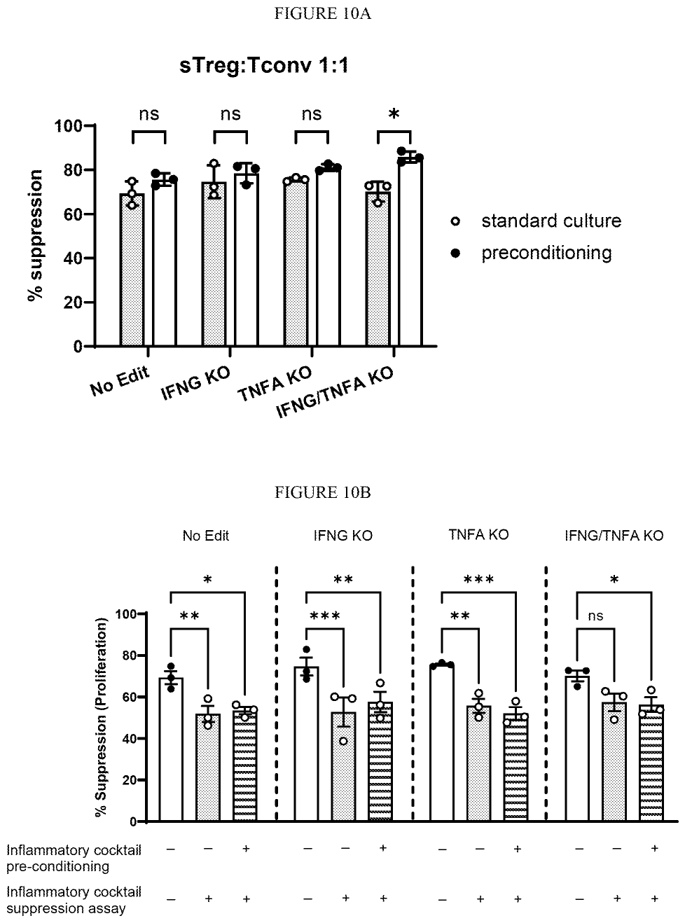

A mixed lymphocyte reaction was used to assay the suppressive function of transduced cells as in Example 2 at a 1:1 ratio of CTV-labelled T cells to engineered T cells, with or without the addition of 100 ng/mL each of TNF-α, IL-1β, and TL-6. Data is are shown in Tables 19 and 20, and in FIG. 10A-B.

TABLE 19
Percent suppression of cell proliferation by engineered
T cells as measured by CTV dilution in suppression
assay without inflammatory cytokines

| | No Pre-conditioning | | Inflammatory Pre-conditioning | |
|---|---|---|---|---|
| Sample | Mean | SEM | Mean | SEM |
| No Edit | 69.4 | 3.1 | 75.6 | 1.6 |
| IFNG KO | 74.6 | 4.3 | 78.5 | 2.6 |
| TNF KO | 75.6 | 0.5 | 81.1 | 0.9 |
| TNF IFNG KO | 70.2 | 2.6 | 85.8 | 1.4 |

TABLE 20
Percent suppression of cell proliferation by engineered T cells as measured
by CTV dilution in suppression assay with and without inflammatory cytokines

| | | | | | Pre-conditioning and | |
|---|---|---|---|---|---|---|
| | | | Inflammatory | | Inflammatory Cytokines in | |
| | Control | | Cytokines in MLR | | MLR | |
| Sample | Mean | SEM | Mean | SEM | Mean | SEM |
| No Edit | 69.4 | 3.1 | 51.8 | 3.8 | 53.4 | 1.8 |
| IFNG KO | 74.6 | 4.3 | 52.7 | 7 | 57.6 | 4.9 |
| TNF KO | 75.6 | 0.5 | 55.7 | 3.4 | 52. | 3.1 |
| TNF IFNG | 70.2 | 2.6 | 57.4 | 4.2 | 56.4 | 3.5 |
| KO | | | | | | |

### Example 6. Suppressive Ability of Engineered T Cells in Inflammatory Bowel Disease Model

Tregs are known to suppress the induction of colitis in pre-clinical models. Moreover, mutations in IL-10 are known to be a risk-factor for the development of colitis in humans. Importantly, inflamed colon is known to express high levels of MAdCAM-1, an adhesion molecule for lymphocytes. T cells overexpressing IL10 and CTLA4, edited to disrupt the genes encoding IFNG and TNFA and further engineered to express an anti-MAdCAM CAR are used for treatment in a humanized mouse model of inflammatory bowel disease (IBD) (see, e.g., Gottel et al., Low-Dose Interleukin-2 Ameliorates Colitis in a Preclinical Humanized Mouse Model. Cell Mol Gastroenterol Hepatol. 2019; 8(2):193-195), or in the CD45RB^{hi} transfer model of IBD (see, e.g., Asseman et al., An Essential Role for Interleukin 10 in the Function of Regulatory T Cells That Inhibit Intestinal Inflammation. J Expt Med. 190(7):995-1003).

Briefly, the humanized mouse IBD model is induced by injecting NOG mice with 20×10⁶ PBMC on day 0 and administering an enema with 50 μL 2,4-dinitrobenzene sulfonic acid (DNBS) (Sigma-Aldrich, Cat. 556971) suspended in a 50% ethanol aqueous solution (X w/v). To evaluate the engineered T cells in a prophylactic setting, 20x10≢engineered T cells provided herein are co-transferred with PBMC from the same donor on Day 0. In a therapeutic setting of the humanized model, PBMCs are injected on the day of the DNBS enema and 20×10\hat{ }6 engineered T cells from the same donor as the PBMCs are transferred on various days following. Following a 20% reduction in body weight, or at a predetermined time for those without loss of at least 20% body weight, mice are euthanized, and their colons collected. Mice are analyzed for the degree of colitis using known methods. For example, the degree of colitis is determined by the total length of the colon as well as the colon length to weight ratio. Colons are fixed in formalin are analyzed by histology for thickening of the colonic epithelium. The prophylactic and therapeutic treatment of mice with engineered T cells with insertion of CTLA4 and IL10 in combination with a knockout of both IFNG and TNFA significantly reduces the thickening of the epithelium. In some embodiments, prophylactic and therapeutic treatment comprises administering engineered T cells with insertion of CTLA4 and a knockout of both IFNG and TNFA and significantly reduces the thickening of the epithelium. In some embodiments, prophylactic and therapeutic treatment comprises administering engineered T cells with insertion of IL10 and a knockout of both IFNG and TNFA and significantly reduces the thickening of the epithelium.

The T helper cell type 1-mediated/CD45RB^{hi} transfer model of IBD is induced by transferring CD45RB^{hi} CD3+CD4+CD25− T cells from BALB/c (Taconic; BALB) into immunodeficient SCID mice (Taconic; CB17SC). Colitis results from the development of a Th1 response, as polarized Th1 cells are presented in intestinal lesions. Co-transfer of the reciprocal CD45RB^{hi} CD4+ T cell subset together with normally pathogenic CD45RB^{high} cells prevents the development of colitis, indicating that the CD45RB^{low} CD4+ subset from normal mice contains a population of regulatory T cells capable of controlling inflammatory responses in the intestine (Powrid et al., Phenotypically distinct subsets of CD4+ T cells induce or protect from chronic intestinal inflammation in C.B-17 scid mice. Int. Immunol. 5:1461-1471). Cell populations are isolated using known methods and prepared for intraperitoneal injection. Engineered Tregs and nTregs are prepared as above in Example 2. Following a 20% reduction in body weight, or at a predetermined time for those without loss of at least 20% body weight, mice are euthanized, and their colons collected. Mice are analyzed for the degree of colitis using known methods such as those provided in the humanized mouse model of IBD. The treatment of mice with engineered T cells with insertion of CTLA4 and IL10 in combination with a knockout of both IFNG and TNFA significantly reduces the thickening of the epithelium by greater than 50%. In some embodiments, prophylactic and therapeutic treatment comprises administering engineered T cells with insertion of CTLA4 and a knockout of both IFNG and TNFA and significantly reduces the thickening of the epithelium by greater than 50%. In some embodiments, prophylactic and therapeutic treatment comprises administering engineered T cells with insertion of IL10 and a knockout of both IFNG and TNFA and significantly reduces the thickening of the epithelium by greater than 50%. Treatment also reduces weight loss, extending viability.

1) An engineered T cell, comprising:
i) a heterologous nucleic acid encoding a regulatory T cell promoting molecule under control of a promoter sequence;
ii) a modification of an endogenous nucleic acid sequence encoding an interferon-gamma (IFNG) wherein the modification knocks down expression of the IFNG; and
iii) a modification of an endogenous a nucleic acid sequence encoding a tumor necrosis factor alpha (TNFA) wherein the modification knocks down expression of TNFA.

2) The engineered T cell of claim 1, wherein the regulatory T cell promoting molecule is a selected from interleukin-10 (IL10), cytotoxic T-lymphocyte associated protein 4 (CTLA4), transforming growth factor beta 1 (TGFB1), indoleamine 2,3-dioxygenase 1 (IDO1), ectonucleoside triphosphate diphosphohydrolase 1 (ENTPD1), 5′-nucleotidase ecto (NT5E), interleulin-22 (IL-22), amphiregulin (AREG), interleukin-35 (IL35), GARP, CD274 molecule (CD274), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), and BTB domain and CNC homolog 2 (BACH2).

3) The engineered T cell of claim 1 or 2, wherein the regulatory T cell promoting molecule is IL10.

4) The engineered T cell of claims 1-2, wherein the regulatory T cell promoting molecule is CTLA4.

5) The engineered T cell of any of claims 1-4, wherein the regulatory T cell promoting molecule is a first regulatory T cell promoting molecule, and further comprising a heterologous nucleic acid encoding a second regulatory T cell promoting molecule under control of a promoter sequence.

6) The engineered T cell of claim 5, wherein the first and the second regulatory T cell promoting molecules are IL10 and CTLA4.

7) The engineered T cell of any one of claims 1-6, further comprising a modification of an endogenous nucleic acid sequence encoding an interleukin 17A (IL17A), an interleukin-2 (IL2), an interleukin 6 (IL6), a perforin 1 (PRF1), a granzyme A (GZMA), or a granzyme B (GZMB), wherein the modification knocks down expression of the IL17A, the IL2, the IL6, the PRF1, the GZMA, or the GZMB, respectively.

8) The engineered T cell of any one of claims 1-7 further comprising a modification of an endogenous nucleic acid sequence encoding an endogenous T cell receptor (TCR), wherein the modification knocks down expression of the endogenous TCR.

9) The engineered T cell of any one of claims 1-8, further comprising a heterologous coding sequence for a targeting receptor under control of a promoter sequence.

10) The engineered T cell of claim 9, wherein the targeting receptor is targeted to a ligand selected from mucosal vascular addressin cell adhesion molecule 1 (MADCAMI1), tumor necrosis factor alpha (TNFA), CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), desmoglein 3 (DSG3), and MHC class I HLA-A (HLA-A*02).

11) The engineered T cell of claim 9 or 10, wherein the targeting receptor comprises a chimeric antigen receptor (CAR) or a T cell receptor (TCR).

12) The engineered T cell of any one of claims 9-11, wherein the heterologous nucleic acid encoding the targeting receptor is incorporated into an expression construct.

13) The engineered T cell of any one of claims 9-12, wherein the heterologous nucleic acid encoding a targeting receptor is in an expression construct that does not comprise a nucleic acid encoding a regulatory T cell promoting molecule.

14) The s engineered T cell of any one of claims 5-13, wherein the heterologous nucleic acid encoding the first regulatory T cell promoting molecule is incorporated into an expression construct and the heterologous nucleic acid encoding the second regulatory T cell promoting molecule is incorporated in an expression construct.

15) The engineered T cell of any one of claims 5-14, wherein the heterologous nucleic acid encoding the first regulatory T cell promoting molecule and the heterologous nucleic acid encoding the second regulatory T cell promoting molecule are incorporated into separate expression constructs.

16) The engineered T cell of claim 13 or 14, wherein the heterologous nucleic acid encoding the first regulatory T cell promoting molecule and the heterologous nucleic acid encoding the second regulatory T cell promoting molecule are incorporated into a single expression construct.

17) The engineered T cell of claim 12 or 14-16, wherein the expression construct further comprises a nucleic acid encoding a targeting receptor.

18) The s engineered T cell of any one of claims 12-17, wherein at least one heterologous coding sequence is in an episomal expression construct.

19) The engineered T cell of any one of claims 1-17, wherein at least one heterologous coding sequence is inserted into the genome.

20) The engineered T cell of claim 19, wherein the insertion into the genome is an untargeted insertion.

21) The engineered T cell of claim 19, wherein the insertion is a targeted insertion.

22) The engineered T cell of claim 21, wherein the targeted insertion is into a site selected from a TCR gene locus, a TNF gene locus, an IFNG gene locus, IL17A gene locus, IL6 gene locus, IL2 gene locus, an adeno-associated virus integration site 1 (AAVS1) locus.

23) The engineered T cell of claim 22, wherein the TCR gene locus is a T cell receptor alpha constant (TRAC) locus.

24) The engineered T cell of any one of claims 1-23, wherein the modification that knocks down expression of a gene comprises one or more of an insertion, a deletion, or a substitution.

25) A population of cells comprising the engineered T cell of any of claims 1-24.

26) A population of engineered T cells of any one of claims 1-24, wherein at least 30%, preferably at least 40%, of cells of the population comprise a heterologous nucleic acid sequence encoding a regulatory T cell promoting molecule under control of a promoter sequence;
at least 50%, preferably at least 70%, of cells of the population comprise a modification of an endogenous nucleic acid sequence encoding an IFNG; and
at least 50%, preferably at least 70%, of cells of the population comprise a modification of an endogenous nucleic acid sequence encoding a TNFA.

27) The population of engineered T cells of claim 26, wherein the percent of cells comprising an insertion or a modification is determined by the percent of reads by next generation sequencing (NGS).

28) The population of engineered T cells of claim 26 or 27, wherein the regulatory T cell promoting molecule is a selected from interleukin-10 (IL10), cytotoxic T-lymphocyte associated protein 4 (CTLA4), transforming growth factor beta 1 (TGFB1), indoleamine 2,3-dioxygenase 1 (IDO1), ectonucleoside triphosphate diphosphohydrolase 1 (ENTPD1), 5′-nucleotidase ecto (NT5E), interleulin-22 (IL22), amphiregulin (AREG), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), and BTB domain and CNC homolog 2 (BACH2).

29) The population of engineered T cells of any one of claims 26-28, wherein the regulatory T cell promoting molecule is IL10.

30) The population of engineered T cell of any one of claims 26-29, wherein the regulatory T cell promoting molecule is CTLA4.

31) The population of engineered T cells of any of claims 26-30, wherein the regulatory T cell promoting molecule is a first regulatory T cell promoting molecule, and further comprising a heterologous nucleic acid encoding a second regulatory T cell promoting molecule under control of a promoter sequence.

32) The engineered T cell of claim 31, wherein the first and the second regulatory T cell promoting molecules are IL10 and CTLA4.

33) The population of engineered T cell of any one of claims 26-32, further comprising a modification of at least one endogenous nucleic acid sequence encoding an interleukin 17A (IL17A), an interleukin 6 (IL6), an interleukin 2 (IL2), a perforin 1 (PRF1), a granzyme A (GZMA), or a granzyme B (GZMB), wherein the population of cells comprises a modification in the at least one of the IL17A, the IL6, the IL2, the PRF1, the GZMA, or the GZMB, respectively, in at least 70% of the population of cells, preferably at least 80% of the population of cells, wherein the modification knocks down expression of the at least one of the IL17A, the IL6, the IL2, the PRF1, the GZMA, or the GZMB, respectively.

34) The population of engineered T cells of any one of claims 26-33, wherein at least 50%, preferably at least 70%, of the cells include a knockdown of a TCR.

35) The population of engineered T cells of any one of claims 26-34, wherein at least 30%, preferably at least 40%, of the cells include an insertion of a nucleic acid coding sequence of a targeting receptor.

36) The population of engineered T cells of claim 35, wherein the targeting receptor binds specifically to a ligand selected from mucosal vascular addressin cell adhesion molecule 1 (MADCAM1), tumor necrosis factor alpha (TNFA), CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), desmoglein 3 (DSG3), and MHC class I HLA-A (HLA-A*02).

37) The population of engineered T cells of claim 35 or 36, wherein the targeting receptor comprises a chimeric antigen receptor (CAR) or a T cell receptor (TCR).

38) The population of engineered T cells of any one of claims 35-37, wherein the heterologous nucleic acid encoding the targeting receptor is incorporated into an expression construct.

39) The population of engineered T cells of claim 38, wherein the heterologous nucleic acid encoding a targeting receptor is in an expression construct that does not comprise a nucleic acid encoding a regulatory T cell promoting molecule.

40) The population of engineered T cells of any one of claims 26-39, wherein the heterologous nucleic acid encoding a first of the at least one regulatory T cell promoting molecule is incorporated into an expression construct and the heterologous nucleic acid encoding a second of the at least one regulatory T cell promoting molecule is incorporated in an expression construct.

41) The population of engineered T cells of claim 40, wherein the heterologous nucleic acid encoding the first regulatory T cell promoting molecule and the heterologous nucleic acid encoding the second regulatory T cell promoting molecule are incorporated into separate expression constructs.

42) The population of engineered T cells of claim 40, wherein the heterologous nucleic acid encoding the first regulatory T cell promoting molecule and the heterologous nucleic acid encoding the second regulatory T cell promoting molecule are incorporated into a single expression construct.

43) The population of engineered T cells of claim 38 or 40-42, wherein an expression construct further comprises a nucleic acid encoding a targeting receptor.

44) The population of engineered T cells of any one of claims 26-43, wherein at least one heterologous coding sequence is in an episomal expression construct.

45) The population of engineered T cells of any one of claims 26-44, wherein at least one heterologous coding sequence is inserted into the genome.

46) The population of engineered T cells of claim 45, wherein the insertion into the genome is an untargeted insertion.

47) The population of engineered T cells of claim 45, wherein the insertion is a targeted insertion.

48) The population of engineered T cells of claim 47, wherein the targeted insertion is into a site selected from a TCR gene locus, a TNF gene locus, an IL2 gene locus, a IL6 gene locus, a IL17A gene locus, an IFNG gene locus, an adeno-associated virus integration site 1 (AAVS1) locus.

49) The population of engineered T cells of claim 48, wherein the TCR gene locus is a T cell receptor alpha constant (TRAC) locus.

50) The population of engineered T cells of claims 26-49, wherein the modification that knocks down expression of a gene comprises one or more of an insertion, a deletion, or a substitution.

51) A pharmaceutical composition comprising any of the engineered T cells of claims 1-24 or population of engineered T cells of claims 25-50.

52) A method or use of administering a cell of any one of claims 1-24 or a population of cells of any one of claims 25-50 or the pharmaceutical composition of claim 51 to a subject.

53) The method or use of claim 52, wherein the subject is in need of immunosuppression

54) The method or use of claim 52 or 53, for treatment of an immune disorder.

55) The method of use of any one of claims 52-54, for treatment of an autoimmune disease.

56) The method or use of claim 55, wherein the autoimmune disease is selected from ulcerative colitis, Crohn's disease, rheumatoid arthritis, psoriasis, multiple sclerosis, systemic lupus erythematosus, and type 1 diabetes.

57) The method or use of any one of claims 52-54, for treatment of graft versus host disease (GvHD).


SEQUENCE LISTING (149 sequences)

SEQ ID NO: 1 - RNA, 20 nt, Artificial Sequence
  source: /note=“Description of Artificial Sequence Synthetic oligonucleotide”
gcaggcaggacaaccauuac

SEQ ID NO: 2 - RNA, 20 nt, Artificial Sequence
  source: /note=“Description of Artificial Sequence Synthetic oligonucleotide”
caggcaggacaaccauuacu

SEQ ID NO: 3 - RNA, 20 nt, Artificial Sequence
  source: /note=“Description of Artificial Sequence Synthetic oligonucleotide”
aggagucagaugcuguuucg

SEQ ID NO: 4 - RNA, 20 nt, Artificial Sequence
  source: /note=“Description of Artificial Sequence Synthetic oligonucleotide”
cuaaaacagggaagcgaaaa

SEQ ID NO: 5 - RNA, 20 nt, Artificial Sequence
  source: /note=“Description of Artificial Sequence Synthetic oligonucleotide”
cgcuucccuguuuuagcugc

SEQ ID NO: 6 - RNA, 20 nt, Artificial Sequence
  source: /note=“Description of Artificial Sequence Synthetic oligonucleotide”
ugucgccagcagcuaaaaca

SEQ ID NO: 7 - RNA, 20 nt, Artificial Sequence
  source: /note=“Description of Artificial Sequence Synthetic oligonucleotide”
cugucgccagcagcuaaaac

SEQ ID NO: 8 - RNA, 20 nt, Artificial Sequence
  source: /note=“Description of Artificial Sequence Synthetic oligonucleotide”
gcgacaguucagccaucacu

SEQ ID NO: 9 - RNA, 20 nt, Artificial Sequence
  source: /note=“Description of Artificial Sequence Synthetic oligonucleotide”
acaugaacucauccaaguga

SEQ ID NO: 10 - RNA, 20 nt, Artificial Sequence
  source: /note=“Description of Artificial Sequence Synthetic oligonucleotide”
guucauguauugcuuugcgu

SEQ ID NO: 11 - RNA, 20 nt, Artificial Sequence
  source: /note=“Description of Artificial Sequence Synthetic oligonucleotide”
gacauucaugucuuccuuga

SEQ ID NO: 12 - RNA, 20 nt, Artificial Sequence
  source: /note=“Description of Artificial Sequence Synthetic oligonucleotide”
ugauggucuccacacucuuu

SEQ ID NO: 13 - RNA, 20 nt, Artificial Sequence
  source: /note=“Description of Artificial Sequence Synthetic oligonucleotide”
aaagaguguggagaccauca

SEQ ID NO: 14 - RNA, 20 nt, Artificial Sequence
  source: /note=“Description of Artificial Sequence Synthetic oligonucleotide”
ccagagcauccaaaagagug

SEQ ID NO: 15 - RNA, 20 nt, Artificial Sequence
  source: /note=“Description of Artificial Sequence Synthetic oligonucleotide”
gauaauggaacucuuuucuu

SEQ ID NO: 16 - RNA, 20 nt, Artificial Sequence
  source: /note=“Description of Artificial Sequence Synthetic oligonucleotide”
cauucagauguagcggauaa

SEQ ID NO: 17 - RNA, 20 nt, Artificial Sequence
  source: /note=“Description of Artificial Sequence Synthetic oligonucleotide”
ugcaggucauucagauguag

SEQ ID NO: 18 - RNA, 20 nt, Artificial Sequence
  source: /note=“Description of Artificial Sequence Synthetic oligonucleotide”
cuucuuuuacauaugggucc

SEQ ID NO: 19 - RNA, 20 nt, Artificial Sequence
  source: /note=“Description of Artificial Sequence Synthetic oligonucleotide”
ugcaucguuuuggguucucu

SEQ ID NO: 20 - RNA, 20 nt, Artificial Sequence
  source: /note=“Description of Artificial Sequence Synthetic oligonucleotide”
uuucagcucugcaucguuuu

SEQ ID NO: 21 - RNA, 20 nt, Artificial Sequence
  source: /note=“Description of Artificial Sequence Synthetic oligonucleotide”
uuuucagcucugcaucguuu

SEQ ID NO: 22 - RNA, 20 nt, Artificial Sequence
  source: /note=“Description of Artificial Sequence Synthetic oligonucleotide”
ugagcacugaaagcaugauc

SEQ ID NO: 23 - RNA, 20 nt, Artificial Sequence
  source: /note=“Description of Artificial Sequence Synthetic oligonucleotide”
ugaaagcaugauccgggacg

SEQ ID NO: 24 - RNA, 20 nt, Artificial Sequence
  source: /note=“Description of Artificial Sequence Synthetic oligonucleotide”
caugauccgggacguggagc

SEQ ID NO: 25 - RNA, 20 nt, Artificial Sequence
  source: /note=“Description of Artificial Sequence Synthetic oligonucleotide”
cuucuuggggagcgccuccu

SEQ ID NO: 26 - RNA, 20 nt, Artificial Sequence
  source: /note=“Description of Artificial Sequence Synthetic oligonucleotide”
gaggcgcuccccaagaagac

SEQ ID NO: 27 - RNA, 20 nt, Artificial Sequence
  source: /note=“Description of Artificial Sequence Synthetic oligonucleotide”
aggcgcuccccaagaagaca

SEQ ID NO: 28 - RNA, 20 nt, Artificial Sequence
  source: /note=“Description of Artificial Sequence Synthetic oligonucleotide”
ggcgcuccccaagaagacag

SEQ ID NO: 29 - RNA, 20 nt, Artificial Sequence
  source: /note=“Description of Artificial Sequence Synthetic oligonucleotide”
gcgcuccccaagaagacagg

SEQ ID NO: 30 - RNA, 20 nt, Artificial Sequence
  source: /note=“Description of Artificial Sequence Synthetic oligonucleotide”
caagaagacaggggggcccc

SEQ ID NO: 31 - RNA, 20 nt, Artificial Sequence
  source: /note=“Description of Artificial Sequence Synthetic oligonucleotide”
ggggccccccugucuucuug

SEQ ID NO: 32 - RNA, 20 nt, Artificial Sequence
  source: /note=“Description of Artificial Sequence Synthetic oligonucleotide”
cuggggccccccugucuucu

SEQ ID NO: 33 - RNA, 20 nt, Artificial Sequence
  source: /note=“Description of Artificial Sequence Synthetic oligonucleotide”
aagcaccgccuggagcccug

SEQ ID NO: 34 - RNA, 20 nt, Artificial Sequence
  source: /note=“Description of Artificial Sequence Synthetic oligonucleotide”
caagcaccgccuggagcccu

SEQ ID NO: 35 - RNA, 20 nt, Artificial Sequence
  source: /note=“Description of Artificial Sequence Synthetic oligonucleotide”
gcugaggaacaagcaccgcc

SEQ ID NO: 36 - RNA, 20 nt, Artificial Sequence
  source: /note=“Description of Artificial Sequence Synthetic oligonucleotide”
ccucuucuccuuccugaucg

SEQ ID NO: 37 - RNA, 20 nt, Artificial Sequence
  source: /note=“Description of Artificial Sequence Synthetic oligonucleotide”
uucuccuuccugaucguggc

SEQ ID NO: 38 - RNA, 20 nt, Artificial Sequence
  source: /note=“Description of Artificial Sequence Synthetic oligonucleotide”
ggcgccugccacgaucagga

SEQ ID NO: 39 - RNA, 20 nt, Artificial Sequence
  source: /note=“Description of Artificial Sequence Synthetic oligonucleotide”
ugguggcgccugccacgauc

SEQ ID NO: 40 - RNA, 20 nt, Artificial Sequence
  source: /note=“Description of Artificial Sequence Synthetic oligonucleotide”
cugcugcacuuuggagugau

SEQ ID NO: 41 - RNA, 20 nt, Artificial Sequence
  source: /note=“Description of Artificial Sequence Synthetic oligonucleotide”
cgaucacuccaaagugcagc

SEQ ID NO: 42 - RNA, 20 nt, Artificial Sequence
  source: /note=“Description of Artificial Sequence Synthetic oligonucleotide”
uuggagugaucggcccccag

SEQ ID NO: 43 - RNA, 20 nt, Artificial Sequence
  source: /note=“Description of Artificial Sequence Synthetic oligonucleotide”
uggagugaucggcccccaga

SEQ ID NO: 44 - RNA, 20 nt, Artificial Sequence
  source: /note=“Description of Artificial Sequence Synthetic oligonucleotide”
aggcacucaccucuucccuc

SEQ ID NO: 45 - RNA, 20 nt, Artificial Sequence
  source: /note=“Description of Artificial Sequence Synthetic oligonucleotide”
ugauuagagagaggucccug

SEQ ID NO: 46 - RNA, 20 nt, Artificial Sequence
  source: /note=“Description of Artificial Sequence Synthetic oligonucleotide”
cugauuagagagaggucccu

SEQ ID NO: 47 - RNA, 20 nt, Artificial Sequence
  source: /note=“Description of Artificial Sequence Synthetic oligonucleotide”
gcugauuagagagagguccc

SEQ ID NO: 48 - RNA, 20 nt, Artificial Sequence
  source: /note=“Description of Artificial Sequence Synthetic oligonucleotide”
ccucucucuaaucagcccuc

SEQ ID NO: 49 - RNA, 20 nt, Artificial Sequence
  source: /note=“Description of Artificial Sequence Synthetic oligonucleotide”
ccagagggcugauuagagag

SEQ ID NO: 50 - RNA, 20 nt, Artificial Sequence
  source: /note=“Description of Artificial Sequence Synthetic oligonucleotide”
ucuaaucagcccucuggccc

SEQ ID NO: 51 - RNA, 20 nt, Artificial Sequence
  source: /note=“Description of Artificial Sequence Synthetic oligonucleotide”
uacugacugccugggccaga

SEQ ID NO: 52 - RNA, 20 nt, Artificial Sequence
  source: /note=“Description of Artificial Sequence Synthetic oligonucleotide”
uuacugacugccugggccag

SEQ ID NO: 53 - RNA, 20 nt, Artificial Sequence
  source: /note=“Description of Artificial Sequence Synthetic oligonucleotide”
gagacacuuacugacugccu

SEQ ID NO: 54 - RNA, 20 nt, Artificial Sequence
  source: /note=“Description of Artificial Sequence Synthetic oligonucleotide”
ggagacacuuacugacugcc

SEQ ID NO: 55 - RNA, 20 nt, Artificial Sequence
  source: /note=“Description of Artificial Sequence Synthetic oligonucleotide”
gggcuacaggcuugucacuc

SEQ ID NO: 56 - RNA, 20 nt, Artificial Sequence
  source: /note=“Description of Artificial Sequence Synthetic oligonucleotide”
ugggcuacaggcuugucacu

SEQ ID NO: 57 - RNA, 20 nt, Artificial Sequence
  source: /note=“Description of Artificial Sequence Synthetic oligonucleotide”
uuaccuacaacaugggcuac

SEQ ID NO: 58 - RNA, 20 nt, Artificial Sequence
  source: /note=“Description of Artificial Sequence Synthetic oligonucleotide”
agagcucuuaccuacaacau

SEQ ID NO: 59 - RNA, 20 nt, Artificial Sequence
  source: /note=“Description of Artificial Sequence Synthetic oligonucleotide”
cagagcucuuaccuacaaca

SEQ ID NO: 60 - RNA, 20 nt, Artificial Sequence
  source: /note=“Description of Artificial Sequence Synthetic oligonucleotide”
uccagcaaacccucaagcug

SEQ ID NO: 61 - RNA, 20 nt, Artificial Sequence
  source: /note=“Description of Artificial Sequence Synthetic oligonucleotide”
cagcaaacccucaagcugag

SEQ ID NO: 62 - RNA, 20 nt, Artificial Sequence
  source: /note=“Description of Artificial Sequence Synthetic oligonucleotide”
ggagcugccccucagcuuga

SEQ ID NO: 63 - RNA, 20 nt, Artificial Sequence
  source: /note=“Description of Artificial Sequence Synthetic oligonucleotide”
uggagcugccccucagcuug

SEQ ID NO: 64 - RNA, 20 nt, Artificial Sequence
  source: /note=“Description of Artificial Sequence Synthetic oligonucleotide”
agcuccaguggcugaaccgc

SEQ ID NO: 65 - RNA, 20 nt, Artificial Sequence
  source: /note=“Description of Artificial Sequence Synthetic oligonucleotide”
gcuccaguggcugaaccgcc

SEQ ID NO: 66 - RNA, 20 nt, Artificial Sequence
  source: /note=“Description of Artificial Sequence Synthetic oligonucleotide”
uggcccggcgguucagccac

SEQ ID NO: 67 - RNA, 20 nt, Artificial Sequence
  source: /note=“Description of Artificial Sequence Synthetic oligonucleotide”
ccgccgggccaaugcccucc

SEQ ID NO: 68 - RNA, 20 nt, Artificial Sequence
  source: /note=“Description of Artificial Sequence Synthetic oligonucleotide”
ccaggagggcauuggcccgg

SEQ ID NO: 69 - RNA, 20 nt, Artificial Sequence
  source: /note=“Description of Artificial Sequence Synthetic oligonucleotide”
gccaaugcccuccuggccaa

SEQ ID NO: 70 - RNA, 20 nt, Artificial Sequence
  source: /note=“Description of Artificial Sequence Synthetic oligonucleotide”
gccauuggccaggagggcau

SEQ ID NO: 71 - RNA, 20 nt, Artificial Sequence
  source: /note=“Description of Artificial Sequence Synthetic oligonucleotide”
ugcccuccuggccaauggcg

SEQ ID NO: 72 - RNA, 20 nt, Artificial Sequence
  source: /note=“Description of Artificial Sequence Synthetic oligonucleotide”
gcuccacgccauuggccagg

SEQ ID NO: 73 - RNA, 20 nt, Artificial Sequence
  source: /note=“Description of Artificial Sequence Synthetic oligonucleotide”
ucagcuccacgccauuggcc

SEQ ID NO: 74 - RNA, 20 nt, Artificial Sequence
  source: /note=“Description of Artificial Sequence Synthetic oligonucleotide”
aucucucagcuccacgccau

SEQ ID NO: 75 - RNA, 20 nt, Artificial Sequence
  source: /note=“Description of Artificial Sequence Synthetic oligonucleotide”
ggagcugagagauaaccagc

SEQ ID NO: 76 - RNA, 20 nt, Artificial Sequence
  source: /note=“Description of Artificial Sequence Synthetic oligonucleotide”
gcugagagauaaccagcugg

SEQ ID NO: 77 - RNA, 20 nt, Artificial Sequence
  source: /note=“Description of Artificial Sequence Synthetic oligonucleotide”
ccagcugguggugccaucag

SEQ ID NO: 78 - RNA, 20 nt, Artificial Sequence
  source: /note=“Description of Artificial Sequence Synthetic oligonucleotide”
cagcugguggugccaucaga

SEQ ID NO: 79 - RNA, 20 nt, Artificial Sequence
  source: /note=“Description of Artificial Sequence Synthetic oligonucleotide”
augagguacaggcccucuga

SEQ ID NO: 80 - RNA, 20 nt, Artificial Sequence
  source: /note=“Description of Artificial Sequence Synthetic oligonucleotide”
ccugggaguagaugagguac

SEQ ID NO: 81 - RNA, 20 nt, Artificial Sequence
  source: /note=“Description of Artificial Sequence Synthetic oligonucleotide”
uacucccagguccucuucaa

SEQ ID NO: 82 - RNA, 20 nt, Artificial Sequence
  source: /note=“Description of Artificial Sequence Synthetic oligonucleotide”
cuuggcccuugaagaggacc

SEQ ID NO: 83 - RNA, 20 nt, Artificial Sequence
  source: /note=“Description of Artificial Sequence Synthetic oligonucleotide”
ggcgaugcggcugauggugu

SEQ ID NO: 84 - RNA, 20 nt, Artificial Sequence
  source: /note=“Description of Artificial Sequence Synthetic oligonucleotide”
cggcgaugcggcugauggug

SEQ ID NO: 85 - RNA, 20 nt, Artificial Sequence
  source: /note=“Description of Artificial Sequence Synthetic oligonucleotide”
ggagacggcgaugcggcuga

SEQ ID NO: 86 - RNA, 20 nt, Artificial Sequence
  source: /note=“Description of Artificial Sequence Synthetic oligonucleotide”
ucugguaggagacggcgaug

SEQ ID NO: 87 - RNA, 20 nt, Artificial Sequence
  source: /note=“Description of Artificial Sequence Synthetic oligonucleotide”
cgccgucuccuaccagacca

SEQ ID NO: 88 - RNA, 20 nt, Artificial Sequence
  source: /note=“Description of Artificial Sequence Synthetic oligonucleotide”
gagguugaccuuggucuggu

SEQ ID NO: 89 - RNA, 20 nt, Artificial Sequence
  source: /note=“Description of Artificial Sequence Synthetic oligonucleotide”
ggcagagaggagguugaccu

SEQ ID NO: 90 - RNA, 20 nt, Artificial Sequence
  source: /note=“Description of Artificial Sequence Synthetic oligonucleotide”
ccaucaagagccccugccag

SEQ ID NO: 91 - RNA, 20 nt, Artificial Sequence
  source: /note=“Description of Artificial Sequence Synthetic oligonucleotide”
caucaagagccccugccaga

SEQ ID NO: 92 - RNA, 20 nt, Artificial Sequence
  source: /note=“Description of Artificial Sequence Synthetic oligonucleotide”
cuggggucucccucuggcag

SEQ ID NO: 93 - RNA, 20 nt, Artificial Sequence
  source: /note=“Description of Artificial Sequence Synthetic oligonucleotide”
ucuggggucucccucuggca

SEQ ID NO: 94 - RNA, 20 nt, Artificial Sequence
  source: /note=“Description of Artificial Sequence Synthetic oligonucleotide”
gaugggcucauaccagggcu

SEQ ID NO: 95 - RNA, 20 nt, Artificial Sequence
  source: /note=“Description of Artificial Sequence Synthetic oligonucleotide”
cugguaugagcccaucuauc

SEQ ID NO: 96 - RNA, 20 nt, Artificial Sequence
  source: /note=“Description of Artificial Sequence Synthetic oligonucleotide”
agauagaugggcucauacca

SEQ ID NO: 97 - RNA, 20 nt, Artificial Sequence
  source: /note=“Description of Artificial Sequence Synthetic oligonucleotide”
ugguaugagcccaucuaucu

SEQ ID NO: 98 - RNA, 20 nt, Artificial Sequence
  source: /note=“Description of Artificial Sequence Synthetic oligonucleotide”
cagauagaugggcucauacc

SEQ ID NO: 99 - RNA, 20 nt, Artificial Sequence
  source: /note=“Description of Artificial Sequence Synthetic oligonucleotide”
augagcccaucuaucuggga

SEQ ID NO: 100 - RNA, 20 nt, Artificial Sequence
  source: /note=“Description of Artificial Sequence Synthetic oligonucleotide”
ugagcccaucuaucugggag

SEQ ID NO: 101 - RNA, 20 nt, Artificial Sequence
  source: /note=“Description of Artificial Sequence Synthetic oligonucleotide”
aagaccccucccagauagau

SEQ ID NO: 102 - RNA, 20 nt, Artificial Sequence
  source: /note=“Description of Artificial Sequence Synthetic oligonucleotide”
gucggucacccuucuccagc

SEQ ID NO: 103 - RNA, 20 nt, Artificial Sequence
  source: /note=“Description of Artificial Sequence Synthetic oligonucleotide”
gacucagcgcugagaucaau

SEQ ID NO: 104 - RNA, 20 nt, Artificial Sequence
  source: /note=“Description of Artificial Sequence Synthetic oligonucleotide”
gauugaucucagcgcugagu

SEQ ID NO: 105 - RNA, 20 nt, Artificial Sequence
  source: /note=“Description of Artificial Sequence Synthetic oligonucleotide”
ucggcaaagucgagauaguc

SEQ ID NO: 106 - RNA, 20 nt, Artificial Sequence
  source: /note=“Description of Artificial Sequence Synthetic oligonucleotide”
cucggcaaagucgagauagu

SEQ ID NO: 107 - RNA, 20 nt, Artificial Sequence
  source: /note=“Description of Artificial Sequence Synthetic oligonucleotide”
uaucucgacuuugccgaguc

SEQ ID NO: 108 - RNA, 20 nt, Artificial Sequence
  source: /note=“Description of Artificial Sequence Synthetic oligonucleotide”
aucucgacuuugccgagucu

SEQ ID NO: 109 - RNA, 20 nt, Artificial Sequence
  source: /note=“Description of Artificial Sequence Synthetic oligonucleotide”
cgacuuugccgagucugggc

SEQ ID NO: 110 - RNA, 20 nt, Artificial Sequence
  source: /note=“Description of Artificial Sequence Synthetic oligonucleotide”
gagucugggcaggucuacuu

SEQ ID NO: 111 - RNA, 20 nt, Artificial Sequence
  source: /note=“Description of Artificial Sequence Synthetic oligonucleotide”
agucugggcaggucuacuuu

SEQ ID NO: 112 - RNA, 20 nt, Artificial Sequence
  source: /note=“Description of Artificial Sequence Synthetic oligonucleotide”
aaaguagaccugcccagacu

SEQ ID NO: 113 - RNA, 20 nt, Artificial Sequence
  source: /note=“Description of Artificial Sequence Synthetic oligonucleotide”
ggauguucguccuccucaca

SEQ ID NO: 114 - RNA, 4140 nt, Artificial Sequence
  source: /note=“Description of Artificial Sequence Synthetic polynucleotide”
auggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaagguguga

SEQ ID NO: 115 - protein, 1379 aa, Artificial Sequence
  source: /note=“Description of Artificial Sequence Synthetic polypeptide”
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 116 - RNA, 4140 nt, Artificial Sequence
  source: /note=“Description of Artificial Sequence Synthetic polynucleotide”
auggacaagaaguacagcaucggacuggacaucggaacaaacagcgucggaugggcagucaucacagacgaauacaaggucccgagcaagaaguucaagguccugggaaacacagacagacacagcaucaagaagaaccugaucggagcacugcuguucgacagcggagaaacagcagaagcaacaagacugaagagaacagcaagaagaagauacacaagaagaaagaacagaaucugcuaccugcaggaaaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguacccgacaaucuaccaccugagaaagaagcuggucgacagcacagacaaggcagaccugagacugaucuaccuggcacuggcacacaugaucaaguucagaggacacuuccugaucgaaggagaccugaacccggacaacagcgacgucgacaagcuguucauccagcugguccagacauacaaccagcuguucgaagaaaacccgaucaacgcaagcggagucgacgcaaaggcaauccugagcgcaagacugagcaagagcagaagacuggaaaaccugaucgcacagcugccgggagaaaagaagaacggacuguucggaaaccugaucgcacugagccugggacugacaccgaacuucaagagcaacuucgaccuggcagaagacgcaaagcugcagcugagcaaggacacauacgacgacgaccuggacaaccugcuggcacagaucggagaccaguacgcagaccuguuccuggcagcaaagaaccugagcgacgcaauccugcugagcgacauccugagagucaacacagaaaucacaaaggcaccgcugagcgcaagcaugaucaagagauacgacgaacaccaccaggaccugacacugcugaaggcacuggucagacagcagcugccggaaaaguacaaggaaaucuucuucgaccagagcaagaacggauacgcaggauacaucgacggaggagcaagccaggaagaauucuacaaguucaucaagccgauccuggaaaagauggacggaacagaagaacugcuggucaagcugaacagagaagaccugcugagaaagcagagaacauucgacaacggaagcaucccgcaccagauccaccugggagaacugcacgcaauccugagaagacaggaagacuucuacccguuccugaaggacaacagagaaaagaucgaaaagauccugacauucagaaucccguacuacgucggaccgcuggcaagaggaaacagcagauucgcauggaugacaagaaagagcgaagaaacaaucacaccguggaacuucgaagaagucgucgacaagggagcaagcgcacagagcuucaucgaaagaaugacaaacuucgacaagaaccugccgaacgaaaagguccugccgaagcacagccugcuguacgaauacuucacagucuacaacgaacugacaaaggucaaguacgucacagaaggaaugagaaagccggcauuccugagcggagaacagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaacauaccacgaccugcugaagaucaucaaggacaaggacuuccuggacaacgaagaaaacgaagacauccuggaagacaucguccugacacugacacuguucgaagacagagaaaugaucgaagaaagacugaagacauacgcacaccuguucgacgacaaggucaugaagcagcugaagagaagaagauacacaggauggggaagacugagcagaaagcugaucaacggaaucagagacaagcagagcggaaagacaauccuggacuuccugaagagcgacggauucgcaaacagaaacuucaugcagcugauccacgacgacagccugacauucaaggaagacauccagaaggcacaggucagcggacagggagacagccugcacgaacacaucgcaaaccuggcaggaagcccggcaaucaagaagggaauccugcagacagucaaggucgucgacgaacuggucaaggucaugggaagacacaagccggaaaacaucgucaucgaaauggcaagagaaaaccagacaacacagaagggacagaagaacagcagagaaagaaugaagagaaucgaagaaggaaucaaggaacugggaagccagauccugaaggaacacccggucgaaaacacacagcugcagaacgaaaagcuguaccuguacuaccugcagaacggaagagacauguacgucgaccaggaacuggacaucaacagacugagcgacuacgacgucgaccacaucgucccgcagagcuuccugaaggacgacagcaucgacaacaagguccugacaagaagcgacaagaacagaggaaagagcgacaacgucccgagcgaagaagucgucaagaagaugaagaacuacuggagacagcugcugaacgcaaagcugaucacacagagaaaguucgacaaccugacaaaggcagagagaggaggacugagcgaacuggacaaggcaggauucaucaagagacagcuggucgaaacaagacagaucacaaagcacgucgcacagauccuggacagcagaaugaacacaaaguacgacgaaaacgacaagcugaucagagaagucaaggucaucacacugaagagcaagcuggucagcgacuucagaaaggacuuccaguucuacaaggucagagaaaucaacaacuaccaccacgcacacgacgcauaccugaacgcagucgucggaacagcacugaucaagaaguacccgaagcuggaaagcgaauucgucuacggagacuacaaggucuacgacgucagaaagaugaucgcaaagagcgaacaggaaaucggaaaggcaacagcaaaguacuucuucuacagcaacaucaugaacuucuucaagacagaaaucacacuggcaaacggagaaaucagaaagagaccgcugaucgaaacaaacggagaaacaggagaaaucgucugggacaagggaagagacuucgcaacagucagaaagguccugagcaugccgcaggucaacaucgucaagaagacagaaguccagacaggaggauucagcaaggaaagcauccugccgaagagaaacagcgacaagcugaucgcaagaaagaaggacugggacccgaagaaguacggaggauucgacagcccgacagucgcauacagcguccuggucgucgcaaaggucgaaaagggaaagagcaagaagcugaagagcgucaaggaacugcugggaaucacaaucauggaaagaagcagcuucgaaaagaacccgaucgacuuccuggaagcaaagggauacaaggaagucaagaaggaccugaucaucaagcugccgaaguacagccuguucgaacuggaaaacggaagaaagagaaugcuggcaagcgcaggagaacugcagaagggaaacgaacuggcacugccgagcaaguacgucaacuuccuguaccuggcaagccacuacgaaaagcugaagggaagcccggaagacaacgaacagaagcagcuguucgucgaacagcacaagcacuaccuggacgaaaucaucgaacagaucagcgaauucagcaagagagucauccuggcagacgcaaaccuggacaagguccugagcgcauacaacaagcacagagacaagccgaucagagaacaggcagaaaacaucauccaccuguucacacugacaaaccugggagcaccggcagcauucaaguacuucgacacaacaaucgacagaaagagauacacaagcacaaaggaaguccuggacgcaacacugauccaccagagcaucacaggacuguacgaaacaagaaucgaccugagccagcugggaggagacggaggaggaagcccgaagaagaagagaaaggucuag

SEQ ID NO: 117 - RNA, 4197 nt, Artificial Sequence
  source: /note=“Description of Artificial Sequence Synthetic polynucleotide”
auggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaagguguccgaguccgccacccccgaguccguguccggcuggcggcuguucaagaagaucuccuga

SEQ ID NO: 118 - protein, 1398 aa, Artificial Sequence
  source: /note=“Description of Artificial Sequence Synthetic polypeptide”
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 119 - RNA, 100 nt, Artificial Sequence
  source: /note=“Description of Artificial Sequence Synthetic polynucleotide”
ccagagcauccaaaagagugguuuuagagcuagaaauagcaaguuaaaauaaggcuaguccguuaucaacuugaaaaaguggcaccgagucggugcuuuu

SEQ ID NO: 120 - RNA, 100 nt, Artificial Sequence
  source: /note=“Description of Artificial Sequence Synthetic polynucleotide”
agagcucuuaccuacaacauguuuuagagcuagaaauagcaaguuaaaauaaggcuaguccguuaucaacuugaaaaaguggcaccgagucggugcuuuu

SEQ ID NO: 121 - RNA, 100 nt, Artificial Sequence
  source: /note=“Description of Artificial Sequence Synthetic polynucleotide”
gaucacgucggccguuggcgguuuuagagcuagaaauagcaaguuaaaauaaggcuaguccguuaucaacuugaaaaaguggcaccgagucggugcuuuu

SEQ ID NO: 122 - DNA, 534 bp, Homo sapiens
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 123 - DNA, 534 bp, Artificial Sequence
  source: /note=“Description of Artificial Sequence Synthetic polynucleotide”
atgcacagctcagcactgctctgttgcctggtcctcctgactggggtgagggccagcccaggccagggcacccagtctgagaacagctgcacccacttcccaggcatcctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgatctccctgggggagaacctgaataccctcaggctgaggctacggcgctgtcatcgactccttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 124 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 125 - protein, 178 aa, Artificial Sequence
  source: /note=“Description of Artificial Sequence Synthetic polypeptide”
MHSSALLCCLVLLTGVRASPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENKSKAVEQVKNAFNKLQEKGIYKAMSEFDIFINYIEAYMTMKIRN

SEQ ID NO: 126 - DNA, 669 bp, Homo sapiens
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaagcgacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 127 - DNA, 669 bp, Artificial Sequence
  source: /note=“Description of Artificial Sequence Synthetic polynucleotide”
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaatacacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatgaggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 128 - DNA, 669 bp, Artificial Sequence
  source: /note=“Description of Artificial Sequence Synthetic polynucleotide”
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaacatacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 129 - DNA, 669 bp, Artificial Sequence
  source: /note=“Description of Artificial Sequence Synthetic polynucleotide”
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggacatcacacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 130 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 131 - protein, 223 aa, Artificial Sequence
  source: /note=“Description of Artificial Sequence Synthetic polypeptide”
MACLGFQRHKAQLNLATRTWPCTLLFFLLFIPVFCKAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 132 - protein, 223 aa, Artificial Sequence
  source: /note=“Description of Artificial Sequence Synthetic polypeptide”
MACLGFQRHKAQLNLATRTWPCTLLFFLLFIPVFCKAMHVAQPAVVLASSRGIASFVCEYASPGKHTEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 133 - protein, 223 aa, Artificial Sequence
  source: /note=“Description of Artificial Sequence Synthetic polypeptide”
MACLGFQRHKAQLNLATRTWPCTLLFFLLFIPVFCKAMHVAQPAVVLASSRGIASFVCEYASPGHHTEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 134 - RNA, 22 nt, Artificial Sequence
  source: /note=“Description of Artificial Sequence Synthetic oligonucleotide”
guuuuagagcuaugcuguuuug

SEQ ID NO: 135 - RNA, 80 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugcuuuu

SEQ ID NO: 136 - RNA, 76 nt, Artificial Sequence
  source: /note=“Description of Artificial Sequence Synthetic oligonucleotide”
guuuuagagcuagaaauagcaaguuaaaauaaggcuaguccguuaucaacuugaaaaaguggcaccgagucggugc

SEQ ID NO: 137 - DNA, 672 bp, Homo sapiens
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaagcgacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaactga

SEQ ID NO: 138 - DNA, 537 bp, Homo sapiens
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 139 - DNA, 1299 bp, Homo sapiens
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgataa

SEQ ID NO: 140 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 141 - DNA, 8028 bp, Artificial Sequence
  source: /note=“Description of Artificial Sequence Synthetic polynucleotide”
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagtcttcttttttgaagacacttcggactgtagaactctgaacctcgagcaatttaaaagaaaaggggggattggggggtacagtgcaggggaaagaatagtagacataatagcaacagacatacaaactaaagaattacaaaaacaaattacaaaaattcaaaatttctgcgttgttgtcggtgctcgttctctgctcttcacgctactgaattcatcaccggttcttcgaaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtctagacgccaccatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggggatccgagggcagaggaagccttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaatctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctccgccttcgccctcagacgagtcggatctctctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 142 - DNA, 7127 bp, Artificial Sequence
  source: /note=“Description of Artificial Sequence Synthetic polynucleotide”
gcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcgaatgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccctgatcattgtcgatcctaccatccactcgacacacccgccagggccctgccaagcttccgagctctcgatatcaaaggaggtacccaacatggtcagcaagggcgaggaactgttcaccggggtggtgcccatcctggtcgagctggacggcgacgtaaacggccacaagttcagcgtgtccggcgagggcgagggcgatgccacctacggcaagctgaccctgaagttcatctgtaccaccggcaagctgcccgtgccctggcccaccctcgtgaccaccctgacctacggcgtgcaatgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcaactcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagaagttgtctcctcctgcactgactgactgatacaatcgatttctggatccgcaggcctctgctagaagttgtctcctcctgcactgactgactgatacaatcgatttctggatccgcaggcctctgctagcttgactgactgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaac

SEQ ID NO: 143 - protein, 7 aa, Artificial Sequence
  source: /note=“Description of Artificial Sequence Synthetic peptide”
PKKKRKV

SEQ ID NO: 144 - protein, 7 aa, Artificial Sequence
  source: /note=“Description of Artificial Sequence Synthetic peptide”
PKKKRRV

SEQ ID NO: 145 - protein, 16 aa, Artificial Sequence
  source: /note=“Description of Artificial Sequence Synthetic peptide”
KRPAATKKAGQAKKKK

SEQ ID NO: 146 - RNA, 100 nt, Artificial Sequence
  source: /note=“Description of Artificial Sequence Synthetic polynucleotide”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 147 - RNA, 100 nt, Artificial Sequence; identical to SEQ ID NO: 146
  source: /note=“Description of Artificial Sequence Synthetic polynucleotide”
  misc_feature (1)..(100): /note=“This sequence may encompass 20, 30, 40, 50, 60, 70, 80, 90, or 100 nucleotides”

SEQ ID NO: 148 - RNA, 300 nt, Artificial Sequence
  source: /note=“Description of Artificial Sequence Synthetic polynucleotide”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 149 - RNA, 100 nt, Artificial Sequence; identical to SEQ ID NO: 146
  source: /note=“Description of Artificial Sequence Synthetic polynucleotide”
  misc_feature (1)..(100): /note=“This sequence may encompass 95-100 nucleotides”